(19) United States
(12) Patent Application Publication
(10) Pub. No.: US 2010/0168228 A1
(43) Pub. Date: Jul. 1, 2010

(54) NOVEL CHEMOTHERAPEUTIC AGENTS AGAINST INFLAMMATION AND CANCER

(75) Inventors: Julie Saha Bose, Maharashtra (IN); Vijay Gangan, Maharashtra (IN)

Correspondence Address: MINTZ, LEVIN, COHN, FERRIS, GLOVSKY AND POPEO, P.C, One Financial Center, BOSTON, MA, 02111, US

(73) Assignee: RELIANCE LIFE SCIENCES PVT. LTD., Navi Mumbai, Maharashtra (IN)

(21) Appl. No.: 12/528,785

(22) PCT Filed: Oct. 12, 2007
(86) PCT No.: PCT/IN07/00488

§ 371 Date: Mar. 15, 2010

(30) Foreign Application Priority Data
Oct. 13, 2006 (IN) 1696/MUM/2006

(51) Int. Cl. A61K 31/343; A61K 31/235; C07D 307/78; C07C 69/732; A61P 35/00; A61P 29/00

(52) U.S. Cl. 514/469; 514/532; 514/533; 514/544; 549/469; 560/61

(57) ABSTRACT

Novel compounds, their methods of preparation and use in therapies related to cancer and inflammation are provided. Compounds comprise esters of cinnamic acid, vanillic acid and 4-hydroxy cinnamic acid and derivatives and salts thereof. Compounds with novel benzofuran lignan structure as a potent antimitotic agent and inducer of apoptosis are provided. Formulations and methods for treatment of diseases mediated by NF-kappaB are also provided.

### CROSS REFERENCE TO RELATED APPLICATIONS

This application claims benefit of provisional Indian Application No. 1696/MUM/2006, filed Oct. 13, 2006, which is hereby entirely incorporated by reference.

### TECHNICAL FIELD OF THE INVENTION

This invention relates generally to the field of compositions for ameliorating cancer and inflammatory diseases. Specifically, the invention relates to ester derivatives of cinnamic acid, vanillic acid, and 4-hydroxy cinnamic acid as anti-tumor and anti-inflammatory agents. More specifically, the invention provides compositions comprising the novel compounds of the invention and methods of their preparation and administration for use in therapies related to cancer and inflammation. The present invention also relates to compounds with novel benzofuran lignan structure as a potent antitumor agent and inducer of apoptosis.

### BACKGROUND OF THE INVENTION

It is estimated by the World Health Organization that about 10 million new cancer cases are occurring now annually around the world. That number is expected to reach 15 million by the year 2015, with two thirds of these new cases occurring in developing countries (World Health 48:22, 1995). For example, it is estimated that there is about 600,000 new cases of lung cancer per year worldwide; approaching 1 million new cases of breast cancer per year; and for head and neck cancer (the sixth most frequently occurring cancer worldwide) an incidence of 500,000 new cases annually. The National Cancer Institute of the United States estimates the overall annual costs for cancer at $107 billion. Treatment costs account for approximately $40 billion. Breast cancer is one of the most significant diseases that affects women. An estimated 212,920 new cases of invasive breast cancer are expected to occurs among women in the U.S. during 2006. Breast cancer is the most frequently diagnosed cancer in women. An estimated 234,460 new cases of prostate cancer in men, 92,700 (men) and 81,770 (women) new cases of lung cancer, 148,000 new cases of colorectal cancer in men and women are predicted for 2006. Cancer killed 6.7 million people around the world in 2002 and this figure is expected to rise to 10.3 million in 2010. (Cancer Facts and Figures 2006, American Cancer Society, Inc. ©2006)

Inflammation, inducible nitric oxide synthase (iNOS) activity and/or cytokine production has been implicated in a variety of diseases and conditions, including pain (Moore et al., “L-NG-nitro arginine methyl ester exhibits antinociceptive activity in the mouse,” Brit. J. Pharmacol., 102:198-202, 1991; Meller et al., “Production of endogenous nitric oxide and activation of soluble guanylate cyclase are required for N-methyl-D-aspartate-produced facilitation of the nociceptive tail-flick reflex,” Eur. J. Pharmacol., 214:93-96, 1992.; Lee et al., “Nitric oxide mediates Fos expression in the spinal cord induced by mechanical noxious stimulation,” NeuroReport, 3:841-844, 1992) and migraine (Olesen et al., “Nitric oxide is a key molecule in migraine and other vascular headaches,” Trends Pharmacol Sci., 15:149-153, 1994). The role of nitric oxide in inflammation is well established. (Bredt, D. S. and Snyder, S. H. (1994) Ann. Rev. Biochem. 63.175.)

Transcription factors that may be involved in inflammation and tumor promotion are NF-κB (protein Nuclear Factor Kappa B), AP-1, CREB, STAT and GATA-3. Most of the inflammatory genes that are over-expressed in the inflammation encode proinflammatory cytokines, chemokines, adhesion molecules and inflammatory enzymes containing κB sites for NF-κB within their promoters, suggesting that these genes are controlled predominantly by NF-κB (Christman J W, Sadikot R T, Blackwell T S. (2000) Chest 117, 1482-1487.)

The nuclear factor NF-κB transcription factor regulates expression of numerous components of the immune system (Li, Q. and Verma, I. M. (2002). NF-kappaB regulation in the immune system. Nat. Rev. Immunol. 2, 725-734). These include proinflammatory cytokines, chemokines, adhesion molecules and inducible enzymes such as cycloxygenase-2 and inducible nitric oxide synthase, which regulate the innate immune response, as well as proteins that regulate the specific immune response, such as major histocompatibility complex and co-stimulatory molecules crucial to the induction phase of specific immunity, and cytokines like interleukin (IL)-2, IL-12 and interferon-gamma that control lymphocyte proliferation and differentiation. Dysregulation of this transcription factor can thus lead to inflammatory and autoimmune diseases (Yamamoto, Y. and Gaynor, R. B. (2001). Therapeutic potential of inhibition of the NF-kappaB pathway in the treatment of inflammation and cancer. J. Clin. Invest. 107, 135-142). Since NF-κB also regulates the expression of a variety of proteins that inhibit apoptosis and promote cell survival/proliferation, it is also implicated in carcinogenesis (Karin, M., Cao, Y., Greten, F. R. and Li, Z. W. (2002). NF-kappaB in cancer: from innocent bystander to major culprit. Nat. Rev. Cancer 2, 301-310.)

NF-κB is present in all cell types. It is involved in cellular responses to stimuli such as stress, cytokines, free radicals, and bacterial or viral antigens. It is an important mediator of the body's response to infection and the incorrect regulation of NF-κB is associated with the occurrence of cancer and a variety of other diseases. NFκB is present in all cells in a resting state in the cytoplasm. Only when NFκB is activated and translocated to the nucleus, the sequence of events leading to activation is initiated (Yamamoto, Y., and Gaynor, R. B. (2001) Curr Mol Med 1, 287-296; Aggarwal, B. B., Takada, Y., Shishodia, S., Gutierrez, A. M., Oommen, O. V., Ichikawa, Haba, Y., and Kumar, A. (2004) Indian J Exp Biol 42, 341-353.). NF-κB describes various dimeric complexes of members of the Rel protein family, which comprises Rel (c-Rel), RelA (p65), RelB, NF-κB1 (p50 and its precursor p105) and NF-κB2 (p52 and its precursor p100) (Ghosh, S., May, M. J. and Kopp, E. B. (1998). NF-kappa B and Rel proteins: evolutionarily conserved mediators of immune responses. Annu. Rev. Immunol. 16, 225-260). Of the various dimeric combinations, p50-p65 is most common. Binding of most NF-κB complexes to motifs in target promoters assists transcription, but homodimeric complexes of p50 or p52 can repress it.

A family of anchoring-domain containing proteins have been identified that keeps the NF-κB in its inactive state within the nucleus. These include IκBα, IκBβ, IκBγ, and IκKε, bcl-3, p105 and p100. The activation of NF-κB and its associated kinases like IKK is in most cases dependent on the production of reactive oxygen species by various stress stimuli. The broad spectrum of the function of phenolic antioxidants suggests their multiple targets through which they interfere with various cellular functions and protect against pathological lesions such as cancer and inflammatory diseases.

NFκB is activated by a wide variety of different stimuli such as pro inflammatory cytokines, oxidant free radicals, inhaled particles, ultraviolet radiation and bacterial or viral products. (Garg et al., 2002 Leukemia 16, 1053-1068). These stimuli reveal that NF-κB is a common pathway for cellular adaptation to stress. The stimuli include inflammatory cytokines (TNFα, IL 4 etc), immune related stress such as bacterial infection of S. aureus and their products such as lipopolysaccharide (LPS), viruses such as HIV-1 and their products such as hemagglutinin of the flu virus, physiological stress such as ischemia, physical stress such as UV irradiation, environmental hazards such as cigarette, smoke, and many therapeutic drugs such as taxol or haloperidol, apoptotic mediators such as anti Fas, growth factors such as insulin, physiological mediators such as angiotensin II or PAF, oxidative stress such as exposure to hydrogen peroxide etc.

NF-κB regulates expression of a number of genes whose products are involved in tumorigenisis and inflammation (Garg et al., 2002 Leukemia 16, 1053-1068). These include antiapoptotic genes (bcl-2 and bcl-XL), cell cycle regulatory genes (eg. Cyclin D1), proinflammatory genes like Tumor Necrosis Factor (TNF), Interleukin-1 (IL-1), inducible Nitric Oxide synthase (iNOS), matrix metalloproteinase (e.g., MMP-9), urokinase-type plasminogen activator (uPA), and many other chemokines.

NF-κB is associated with the expression of pro-inflammatory genes during the onset of inflammation and with the expression of anti-inflammatory genes during the resolution of inflammation. Inhibition of NF-κB at the onset of inflammation results in decreased inflammatory response. (Lawrence et al Nature Medicine 7:1291 (2001), Transcription factors belonging to the NF-κB family regulate a range of genes that mediate inflammation and cell survival.(Farrow B., Evers B. M., Surg. Oncol., 10, 153-169 (2002)).

NF-κB also regulates the production of prostaglandins via the proinflammatory gene cyclo-oxygenase-2 (COX-2), which has shown to be overexpressed in a variety of cancers including colorectal cancer and mesothelioma (Kalgutkar, A. S., Zhao, Z.(2001) Current Drug Targets 2:79-106; Marrogi, A., et al. (2000) Cancer Res 60:3696-3700). Cyclooxygenase (COX) is involved in the inflammatory process and catalyzes the rate-limiting step in the synthesis of prostaglandins from arachidonic acid. COX exists in two isoforms; COX-1 and COX-2. (Funk C. D., et al., FASEB J., 5, 2304-2312 (1991)). COX-1 is expressed constitutively in most tissues and appears to be responsible for maintaining normal physiological functions whereas COX-2 is detected in only certain types of tissues and is induced transiently and up-regulated by various pro-inflammatory agents, including lipopolysaccharide, cytokines, and growth factors. (Hinz B., Brune K., J. Pharmacol. Exp. Ther., 300, 367-375 (2002).)

Cell cycle aberrations and blocking of apoptosis provide molecular markers of cancer and cell cycle and apoptosis modulators act as targets for cancer prevention. A hallmark of cancer is the accumulation of cells with abnormal cell cycle regulation—cell division may be accelerated, cell death slowed, or a combination of these activities. Cell cycle regulatory targets are key cancer therapy targets and numerous cancer therapies induce apoptosis. Cell cycle is an endpoint that is related to cancer development and can be measured in cultured cells. Other endpoints include antioxidant, cellular signaling, etc, that may result in change in cell cycle. The cell cycle in all eukaryotes is composed of five phases, beginning with G1 phase, followed by the DNA synthesis or “S” phase, then the G2 phase, then mitosis or “M” phase, and finally G0, the quiescent state (Hunter, T. and Pines, J. (1991) Cell 66, 1071-1074). Cyclin:cyclin-dependent kinase complexes control the two critical checkpoints in the cell cycle at the G1/S and G2/M transitions by phosphorylating a variety of proteins, such as nuclear lamins and histones for nuclear membrane breakdown and chromosome condensation, as well as proteins leading to the transcription of genes required for proliferation. (Draetta, G. (1990) Trends in Biol. Sci 15, 378-382).

Currently, there is an increasing interest in therapeutic use of antioxidants to prevent tissue damage induced by overproduction of Reactive Oxygen Inducers (ROI), by reducing free radical formation or by scavenging or promoting the breakdown of these species (Cuzzocrea, S., Riley, O. P., Caputi, A. P., and Salvemini, D. (2001) Pharmacol. Rev. 53, 135-159). Experiments in different in vitro and in vivo systems have demonstrated the potent anti-oxidant action of plant polyphenols (Damianaki, A. et al., (2000) J. Cell. Biochem. 78:429-441), and it has been suggested that they can prevent oxidative stress related diseases (Aucamp, J. et al., (1997) Anticancer Res. 17:4381-4385).

Phenolic photochemical are diverse group of compounds that exhibit anti-inflammatory, antioxidant, anticarcinogenic, anti-diabetic, anti-atherosclerosis and immunomodulatory activities. These phytochemicals are commonly called chemotherapeutics or chemopreventive agents. Human beings consume such phytochemicals from dietary sources, either as natural components or as synthetic food additives. These phytochemicals may fight disease through suppression of the inflammatory response. Dysregulated inflammation is the cause of a great many diseases including cancer and atherosclerosis (Coussens, L. M., and Werb, Z. (2002) Nature 420:860-867.; Balkwill, F., and Mantoxani, A. (2001) Lancet 357, 539-545). It stands to reason, then, that suppression of inflammation, whether by phytochemicals or by other means, should delay the onset of disease (Craig, W. J. (1997) J Am Diet Assoc 97:S199-204; Craig, W. J. (1999) Am J Clin Nutr 70:491S-499S.).

Phenolic compounds widely occur in a variety of plants. Phenolic compounds are ubiquitous in the plant kingdom. These secondary plant metabolites are commonly divided into five major groups, the anthocyanidines, the flavonols/flavones, the flavanones, and the flavan-3-ols and their oligomers and the polymers, the proanthocyanidins. A less common group of flavonoids are chalcones and dihydrochalcones, which are mostly found in individual fruits and vegetables. The fifth group of phenolic compound is hydroxycinnamic acids. The most common hydroxycinnamic acid derivatives are esters of caffeic acid with quinic acid and the caffeic acid phenylethyl ester (Natarajan, K, et al. (1996) Proc. Natl. Acad. Sci. USA 93:9090-9095). Esters of caffeic acid with quinic acid are the main constituent in coffee, apple juice, artichoke, eggplant, peach, cherry, plum, elderberry, apricot etc. The caffeic acid phenyl ester (CAPE) is a structural relative of flavanoids that is an active component of propolis from honeybee hives. It has antiviral, anti-inflammatory, and immunomodulatory properties (Grunberger, D., Banerjee, R., Ersinger, K., Oltz, K., Efros, E. M., Caldwell, M., Esterez, V. and Nakarishi, K. (1988) Experientia 44, 230-232.) and has been shown to inhibit the growth of different types of transformed cells (Grunberger, D., et al. (1988) Experientia 44, 230-232; Burke T R Jr, Fesen M R, Mazumder A, Wang J, Carothers A M, Grunberger D, Driscoll J, Kohn K, Pommier Y. (1995) J. Med Chem 38(21):4171-4178; Su, Z Z, Grunberger, D. and Fisher, P. B. (1991) Mol. Carcinog. 4:231-242; Su, Z Z, Grunberger, D. and Fisher, P. B. (1994) Cancer Res. 54:1865-1870; Hlandon, B., et al. (1980) Arzneim. Forch. 30, 1847-1848; Guarini L, Su Z Z, Zucker S, Lin J, Grunberger D, Fisher P B. (1992) Cell. Mol. Biol. 38:513-527). In the transformed cells, CAPE and phenolic compounds are known to alter the redox state and induce apoptosis (Chiao, C., Carothers, A. M., Grunberger, D., Solomon, G., Preston, G. A. and Barrett, J. C. (1995) Cancer Res 55, 3576-3583). Although some of the polyphenols are considered to be non-nutritive, interest in these compounds has arisen because of their possible beneficial effects on health.

Several mechanisms have been studied for cancer prevention by polyphenolic phytochemical, including modulating cell signaling, inhibiting inflammation, anti-hormone actions, modulating growth factors, antioxidant activities, enhancing apoptosis, and inhibiting cell cycle.

While phenolic acids, such as Caffeic acid (3,4,dihydroxycinnamic acid), Cinnamic acid Ferulic acid, Vanillic acid etc are common in many plant foods, only a few examples of their esters with aromatic alcohols (eg. phenylethyl ferulate, caffeic acid phenylethyl ester (CAPE)) are found to be naturally occurring. The esters have antioxidant (Chen Z H and Ho, C H, J. Agric. Food Chem., 45(7): 2374-2378, 1997), anticancer & anti-inflammatory, anti-HIV (Burke et al., (1995) J. Med Chem 38(21):4171-4178), antimicrobial activities. The role of mediators in inflammatory and cancer have led to the derivation or designing of molecules which involve novel combinations of these natural compounds for functional therapeutic effects.

Chemotherapy is one of the most common treatments for cancer. It is the main treatment for some types of cancer, such as leukemia, Hodgkin's disease and non-Hodgkin's lymphomas. Cancers of the lung, breast, testes, colon, ovary, and stomach are also treated with chemotherapy. For some patients, chemotherapy may be the only treatment they receive. Majority of the chemotherapeutic agents presently used for cancer treatment were developed by screening in a growth inhibition assay that could inhibit tumor cell proliferation. These chemical substances inhibit the growth of a variety of cancer cells, utilizing a remarkable number of diverse mechanisms that include cell cycle arrest, induction of apoptosis, disruption of microtubules, inhibition of angiogenesis, and increasing oxidative damage. Taxols a well-established chemotherapeutic agent used for treating childhood and adult tumors acts by disrupting the microtubule function and causing growth arrest in the G2/M phase of the cell cycle. Thus, chemotherapy becomes effective because the drugs used effect some phase of the cell life cycle. Depending on the drug chosen, chemotherapy can affect malignant cells in one of the three ways: First, damage the DNA of cancer cells so that it can no longer reproduce, thus preventing replication. Second, inhibit the synthesis of new DNA strand so that no cell replication is possible. This is done by blocking the formation of nucleotides that are necessary for new DNA synthesis, hence arresting the cells in S phase. Third, stop the mitotic process by disrupting the microtubule spindle formation.

Apoptosis is the consequence of a series of precisely regulated events that are frequently altered in tumor cells. The mechanism of apoptosis involves a cascade of initiator and effector caspases that are activated sequentially (Kasibhatla, S. (2004) Molecular Cancer Therapeutics 3(11)1365-1373), followed by chromatin condensation, nuclear fragmentation, plasma membrane blebbing and cell shrinkage. Eventually the cells break into small membrane surrounded bodies (apoptotic bodies), which are eaten up by phagocytes without inciting an inflammatory response in the vicinity. Novel and synthetic molecules capable of modulating cell cycle by targeting G2/M checkpoint followed by induction of apoptosis in multidrug-resistance tumors remain compelling for drug discovery in oncology (Li, Q., Sham, H., Rosenberg, S., (1999) Annu Rev Med Chem 34, 139-242; Jordan, A., Hadfield, J. A., Lawrence, N. J., McGown, A. T. (1998) Med Res Rev 18, 259-296). Protein tyrosine phosphorylation is another central signal pathway involved in mediating various cellular processes such as cell cycle progress, transcriptional regulation, cell transformation, proliferation, differentiation and apoptosis (O'Callaghan et al., (2001) Cell Biol. Toxicol 17, 127-137; Kalidas et al., (2001) JAMA 286, 895-898). Several leukemic and breast cancerous cell lines (Sainsbury, J. R. C. et al., (1987) Lancet i:1398-1402) have an elevated phosphotyrosine content suggesting that disruption of balance between phosphorylation and dephosphorylation reactions could have dramatic consequences on normal regulation of cell proliferation.

Recently, a class of dihydro-benzofuran lignans was shown to posses potential antiproliferative and antitumoral activities (Pieters, L., Dyck, S. V., Gao, M., Bai, R., Hamel, E., Vlietinck, A. and Lemiere, G. (1999) J. Medical Chemistry 42, 5475-5481). Synthetic precursors and analogues of benzofuran lignans derivatives were synthesized and explored for their potential antiangiogenic and antitubilin/antimitotic activities in the past (Apers S, Paper D, Burgermeister J, Baronikova S, Van Dyck S, Lemiere G, Vlietinck A, Pieters L. (2002). J. Natural Product 65:718-720; Pieters, L. et. al., 1999).

### OBJECT OF THE INVENTION

It is the object of the present invention to provide novel compounds for anti-inflammatory and for cancer therapy.

It is the object of the present invention to provide derivatives of cinnamic acid as represented by formula I.

It is the object of the present invention to provide derivatives of vannilic acid as represented by formula II.

It is the object of the present invention to provide derivatives of 4-Hydroxy cinnamic acid as represented by formula III.

It is the object of the present invention to provide a novel benozfuran lignans derivatives.

It is the object of the present invention to provide methods of preparation of the novel compounds.

It is the object of the present invention to provide compositions and formulations of the novel compounds.

It is the object of the present invention to determine the potential use of the novel compounds in cancer and inflammation.

It is the object of the present invention to provide mechanisms of action of these compounds as anti inflammatory and chemotherapeutic agents.

### SUMMARY OF THE INVENTION

The present disclosure provides novel compounds, methods, compositions and potential uses for the treatment of cancer and inflammation.

The present invention relates to the combinatorial synthesis of wide variety of novel ester derivatives from known phenolic phytochemicals as represented by Formula I. II and III, and their potential use as antitumor and anticancer agents.

The present invention provides derivatives of Cinnamic acid as represented by the formula (I):

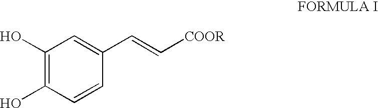

The present invention provides esters of cinnamic acid of formula I wherein R is selected from aryl, hetero aryl groups.

In the preferred embodiments the present invention includes compounds of formula I wherein R is selected from vannilic acid, ferulic acid, eugenol, salicylic acid and/or their derivatives.

The present invention provides derivatives of Vanillic acid as represented by the formula (II):

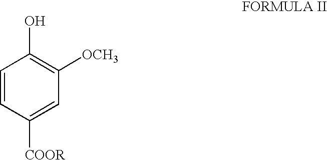

The present invention provides esters of vanillic acid of formula II wherein R is selected from aryl, hetero aryl groups.

In the preferred embodiments the present invention includes compounds of formula II wherein R is selected from vanillic acid, ferulic acid, eugenol, salicylic acid and/or their derivatives.

The present invention also provides esters of 4-hydroxy cinnamic acid as represented in formula

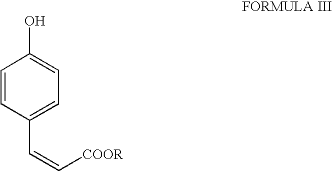

In the preferred embodiments the present invention includes compounds of formula III wherein R is selected from Vanillic acid, ferulic acid, eugenol, salicylic acid, cinnamic acid and/or their derivatives.

In one embodiment, the present invention relates to the compounds of formula I, II, III and their derivatives thereof including but not limited to polymorphs, isomers and prodrugs thereof, geometric or optical isomers thereof, and pharmaceutically acceptable esters, ethers, carbamates of such compounds, all solvates and hydrates thereof and all salts thereof.

The present invention further relates to a novel benzofuran lignan structure as a potent antimitotic agent and inducer of apoptosis. The inventors of the present invention have found that this novel benzofuranlignan structure, efficiently arrests Jurkat T lymphocytes (p53^{+/+}) in the G2/M phase of the cell cycle and induces apoptosis, thus inhibiting cell growth. It is for the first time that the synthesis of novel benzofuran lignan structures have shown potential antitumor/antiproliferative activities.

In one embodiment, the present invention relates to the compounds of benzofuran lignan structures and their derivatives thereof including but not limited to polymorphs, isomers and prodrugs thereof, geometric or optical isomers thereof, and pharmaceutically acceptable esters, ethers, carbamates of such compounds, all solvates and hydrates thereof and all salts thereof.

In one embodiment the present invention provides methods for preparation of the novel compounds, which includes all conventional methods of esterification of one acid with other phenol. The preferred process involves esterification, protection of all hydroxyl groups followed by hydrolysis to get corresponding acid which reacts with phenolic compound to get corresponding fused ester derivative. The deprotection of hydroxyl groups yields the compound of invention which is then purified and characterized by conventional techniques.

In one embodiment, the present invention provides the mechanism of action of the compounds of formula I, II, III, benozfuran lignan molecules and derivatives thereof. The present invention in particular has studied the effect of these molecules on NF kappa B modulation.

In one embodiment the present invention provides the pharmaceutical formulations comprising of any of compound of formulas I, II, III, benozfuran lignan molecules and derivatives thereof alone or in combination with a suitable pharmaceutically acceptable excipients. Such formulations are useful in cancer and inflammation. The compounds of the present invention can be administered alone or in combination with other active ingredients.

In one embodiment the present invention provides the method of treatment of cancer by administering to a subject a therapeutically effective amount of the compounds of formulas I, II, III, benozfuran lignan molecules and their derivatives which can be either given alone or in combination with other therapies.

In one embodiment the present invention provides the method of treating inflammation by administering to a subject a therapeutically effective amount of the compounds of formula I, II, III, benozfuran lignan molecules and their derivatives which can be either given alone or in combination with other therapies.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIG. 1: Effect of CAMVE on LPS induced nitrite production. Raw 264.7 cells were pretreated with indicated concentrations of CAMVE for 1 h before being incubated with LPS (250 ng/ml) for 24 h. The culture supernatant was subsequently isolated and mixed with an equal volume of Griess reagent (1% sulfanilamide, 0.1% naphthylenediamine dihydrochloride, and 2% phosphoric acid) and incubated at room temperature for 15 min. NaNO₂ was used to generate a standard curve, and nitrite production was determined by measuring optical density at 540 nm. Each column shows the mean±S.D. of triplicate determinations.

FIG. 2: Effect of CAMVE on TNF induced ROI generation (A), and Lipid Peroxidation (B). For A, Jurkat cells were pretreated with indicated concentrations of CAMVE for 1 h. After being stimulated with TNF (1 nM) for 4 h, the relative mean fluorescence intensity (MFI) was measured using a FACS Calibur (BD). The results shown are representative of two independent experiments. For B, Jurkat cells were pretreated with indicated concentrations of CAMVE for 3 h and then incubated with TNF (1 nM) for 1 h and assayed for lipid peroxidation, as described in the “Materials and Methods”.

FIG. 3: Effect of CAMVE on TNF or LPS dependant NF-κB activation is dose dependent. Jurkat cells were preincubated at 37° C. for 3 h with indicated concentrations of CAMVE followed by 30 min incubation with 0.1 nM TNF or 100 ng/ml SA-LPS (serum activated LPS). After these treatments, nuclear extracts were prepared and then assayed for NF-κB activation as described in the “Materials and Methods”. In the assay performed, the use of specific antibodies against p65 and p50 subunits of the NF-κB heterodimer bound to it's specific oligo coated wells, confirms that the TNF-activated complex consisted of p50 and p65 subunits of the NF-κB transcription factor.

FIG. 4: Effect of CAMVE on NF-κB activation in different cell lines. A. HeLa, MCF-7, U937, were incubated at 37° C. for 3 h with 15 μM dose of CAMVE and then stimulated with 0.1 nM TNF for 30 min. After these treatments, nuclear extracts were prepared and assayed for NF-κB using BD Transfactor ELISA method. B. RAW 264.7 cells pretreated with indicated doses of CAMVE were stimulated with 100 ng/ml LPS for 30 min and assayed for NF-κB activation.

FIG. 5: Effect of CAMVE on TNF or LPS induced nuclear translocation of p65. A. HeLa cells either untreated or pretreated with 15 μM CAMVE for 3 h at 37° C. were stimulated with 0.1 nM TNF and immunocytochemical analysis were performed as described in “Materials and Methods”. B. Jurkat (5×10⁵ cells/ml) cells were pretreated with indicated concentrations of CAMVE for 3 h and then stimulated with 0.1 nM of TNF for 30 min or 100 ng/ml of SA-LPS (serum activated LPS) for 15 min. After these treatments, cells were washed twice with PBS and nuclei were isolated by incubating the cells with 2000 Pipes-Triton buffer for 30 min at 4° C. Nuclei were stained for p65 and analyzed using FACS. (i). Representative histogram overlay showing TNF or LPS stimulated (1), basal (2), CAMVE stimulated (3), p65 staining in the nucleus. (ii). Ordinates gives the Isotype-corrected MFI for various treatments.

FIG. 6: Effect of CAMVE on TNF or LPS induced COX-2 expression. Cells were pretreated with indicated concentrations of CAMVE for 3 h and then stimulated with either TNF (0.1 nM) or SA-LPS (100 ng/ml) for 12 h. After harvesting the treated cells, the cellular lysates were checked for COX-2 protein expression by an enzyme immunoassay as described in the “Materials and Methods”.

FIG. 7: Effect of CAMVE on TNF or LPS induced ICAM1 (CD54) expression. Cells (5×10⁵ cells/ml) were pretreated with different concentrations of CAMVE for 3 h and then treated with TNF (0.1 nM) or SA-LPS (100 ng/ml) for 12 h at 37° C. in a CO₂ incubator. Treated cells were washed and stained with anti human CD54-FITC conjugated antibody to measure the amount of surface ICAM1 expression. The relative mean fluorescence intensity (MFI) was measured using FACS Calibur (BD). A. Representative histogram overlay showing TNF or LPS stimulated (1), basal (2), CAMVE pretreated (3). B. Values are given as MFI (mean±S.D.) in percent of basal expression, which was set to 100%.

FIG. 8: CAMVE potentiates apoptosis induced by TNF or chemotherapeutic agents. A1. Jurkat cells were incubated at 37° C. with TNF, in the presence and absence of 10 μM of CAMVE for 72 h and the viable cells were assayed using MTT reagent. The results are shown as the mean±S.D. from triplicate culture. A2. Jurkat cells (1×10⁶ cells/ml) were pretreated with CAMVE for 3 h as indicated and incubated with TNF for 24 hrs, and PARP cleavage was determined by FACS analysis as described in the “Materials and Methods”. M₂ gated population represents the percentage apoptotic population B. SA-LPS/Jkt cells pretreated with 10 μM of CAMVE for 3 h, were treated with 1 μM of cis-plastin, doxorubicin, taxol or vincristine for 72 h. The cytotoxicity was then assayed by MTT method. Results are shown as the mean±S.D. from triplicate samples.

FIG. 9: CAMVE induces differential cytotoxicity in different human tumor cell lines. Different human tumor cell lines were cultured either in the presence or absence of CAMVE (30 μM) for 72 h. The MTT assay was done and absorbance taken at 570 nm. The result indicated are mean O.D. of triplicate culture.

FIG. 10: Effect of CAMVE on Cell Cycle distribution. A. 5×10⁵ cells were treated with indicated concentrations of CAMVE as indicated and cell cycle analyses were performed using Flow Cytometry as described in the “Materials and Methods”. The percentage of cells in G1, S, and G2-M phase were calculated using Cell Quest analysis software and are represented within the histograms. The data shown here are from a representative experiment repeated three times with similar results. B. Expression of human Cyclin D1, was assayed by semi quantitative RT-PCR analysis with GAPDH as internal control. Cells were treated for 24 h with various doses of the compound (0, 5, 10 and 15 μM), then total RNA was extracted and submitted to RT-PCR.

FIG. 11: Dose response for compound 27 induced loss of cell viability and cell proliferation in Jurkat cell line. Jurkat cells were treated with 10, 50, 100, 500 nM of the compound, and cell viability was determined by MTT assay 24 h, 48 h after treatment and the GI₅₀ value was estimated. Error bars indicate±S.D.

FIG. 12: Cell Cycle analysis of Jurkat cells after treatment with various doses of the compound stated as compound 27. 5×10⁵ cells were treated with different concentration of the compound for 24 h and after staining with PI, cell cycle distribution was analyzed using Flow Cytometer. The data indicate the percentage of cells in each phase of the cell cycle. All experiments were performed in duplicate and gave similar results.

FIG. 13: Changes in Cell Cycle distribution with time after treatment of Jurkat cells with the compound stated as compound 27. Jurkat cells were treated with 0.1 μM and 0.5 μM of the compound for 24, 48, 72 h and the percentage of cells in the cell cycle phases (G1, S, and G2/M) were analyzed by flow cytometry. Results are expressed as means±S.D.

FIG. 14: Induction of Caspase 3 by compound 27. Jurkat cells were treated with indicated concentrations of the compound for 16 and 24 h and harvested in lysis buffer. Cellular lysates were incubated with Ac-DEVD-pNA as described in the “Materials and Methods” for 2 h at 37° C. Absorbance was recorded at 405 nm.

FIG. 15: Induction of apoptosis and PARP cleavage by compound 27. Jurkat cells were treated with 100 and 500 nM of the compound for indicated time period and PARP cleavage was determined using FACS analysis as described in the “Materials and Methods”. Percentage apoptotic populations are represented as the M₂ gated population.

FIG. 16: Compound 27 induced apoptosis in Jurkat cells. A. Morphological aspects of propidium iodide stained cells. Jurkat cells were treated for 24 h with different concentrations of the compound and stained with propidium iodide. Arrows identify apoptotic or fragmented nuclei. B. Fragmentations of genomic DNA in cells after treatment for 24 h with indicated concentrations of the compound. Fragmented DNA was extracted and analyzed on 2% agarose gel.

FIG. 17: Differential effect of compound 27 on the cell cycle distribution in U937 cell line. Cells were treated with different concentration of the compound for 24 h and after staining with PI, cell cycle distribution was analyzed using Flow Cytometer. The data indicates the percentage of cells in each phase of the cell cycle.

FIG. 18: Effects of compound 27 on p53, Bax, bcl-2 mRNA levels in Jurkat cells. Expression of human p53, bax-α, bcl-2, was assayed by semi quantitative RT-PCR analysis with GAPDH as internal control. Cells were treated for 24 h with various doses of the compound (0, 50, 100 and 500 nM), then total RNA was extracted and submitted to RT-PCR.

FIG. 19: Effect of compound 27 on the apoptosis in cells with different p53 status. Extent of apoptosis in different cells was measured by staining the cells for PARP cleavage followed by FACS analysis. Cells were treated with 100 nM of the compound for 24 h and the level of apoptosis was seen. Percentage apoptotic populations are represented as the M₂ gated population.

FIG. 20: Suppression of phosphotyrosine levels in Jurkat cells by compound 27. 1×10⁶ cells were treated with indicated concentrations of the compound for 24 h. Cells were fixed and permeabilized as described in the “Materials and Methods”, and the extent of tyrosine phosphorylation in the cells was determined by measuring the increase in fluorescence produced by the FITC-labeled monoclonal antibody compared to the FITC-labeled isotype control antibody. 100 nM concentration was sufficient to bring significant reduction in tyrosine phosphorylation levels compared to the control values.

### DETAILED DESCRIPTION OF THE INVENTION

Combinatorial synthesis and subsequent assays for anti-inflammatory and antitumor activities of various novel ester derivatives has been applied in this study. The invention relates to their rational use as modulators of cell signaling and use as chemotherapeutic agents against inflammation and carcinogenic diseases.

The inventors of the present invention have recognized the potential of NF-κB as a therapeutic target, and have focused on preparing novel ester derivatives classified into three categories according to the common phenolic acid present in such class. These are derivatives of cinnamic acid as represented in formula I, derivatives of vannillic acid as represented in formula II and derivatives of hydroxy cinnamic acid as represented by formula III. These novel ester derivatives can suppress NF-κB activation induced by inflammatory agents and carcinogens and block NF-κB regulated gene expression that mediates inflammation and carcinogenesis. It has been surprisingly found by the inventors of the present invention, that these ester derivatives could inhibit NF-κB activation and potentiate apoptosis mediated by chemotherapeutic agents. Further, the inhibition of TNF induced ROI generation and inhibition of NF-κB activated gene expression was also observed. The present invention deals with the combinatorial synthesis of wide variety of novel ester derivatives from known phenolic phytochemicals, and their potential use as antitumor and anticancer agents.

The inventors of the present invention have also discovered a novel benzofuran lignan structure as a potent antimitotic agent and inducer of apoptosis. The inventors of the present invention have noticed that this novel benzofuran lignan structure, efficiently arrests Jurkat T lymphocytes (p53^{+/+}) in the G2/M phase of the cell cycle and induces apoptosis, thus inhibiting cell growth. The protooncogenes, p53 (tumor suppressor gene), bcl-2 (antiapoptotic gene), bax-α (proapoptotic gene), are known to regulate cell cycle and apoptosis (Hale et. al., (1996). Eur. J. Biochem., 236, 1-26). The influence of p53 gene product, a key element in apoptosis and G2/M arrest, has been characterized in depth (Bunz, F. et. al., (1998) Science 282, 1497-1501). In this study, the molecular mechanisms of the antiproliferative and apoptotic effects of our novel molecule were investigated to determine whether the transduction signals and/or genes expression are involved and whether it could also affect the tyrosine phosphorylation status. It is for the first time that the synthesis of novel benzofuran lignan structures have shown potential antitumor and antiproliferative activities.

### DEFINITIONS

The term “compounds” of the invention as used herein refers to the compounds derived from the cinnamic acid, tannic acid and gallic acid, more preferably the esters of these acids as represented by some compounds described in the Table 1.

The term “pharmaceutically acceptable” as used herein refers to the substance including carrier, diluent, vehicle excipient, or composition being compatible chemically and/or toxicologically, with the other ingredients comprising a formulation that is not deleterious to the recipient thereof.

The term “aryl” means an aromatic hydrocarbon group having a single (e.g. phenyl) or a fused ring system (e.g. naphthalene, anthracene, phenanthrene, etc.). A typical aryl group is aromatic carbocylic ring having 6, 7, 8, 9 or 10 carbon atoms, such as phenyl, naphthyl, tetrahydronaphthyl or indenyl, which may optionally be substituted with one or more substituents selected from hydroxy, amino, halogen, nitro, cyano, C₁ to C₄ alkyl, C₂ to C₄ alkenyl, C₂ to C₄ alkynyl, C₁ to C₄ alkoxy, C₁ to C₄ dialkylamino, the alkyl moieties having the same meaning as previously defined. The preferred aromatic hydrocarbon group is phenyl.

The term “heteroaryl” means a substituted or unsubstituted aromatic group having at least including one heteroatom selected from N, O and/or S, like imidazolyl, thiadiazolyl, pyridyl, (benzo)thienyl, (benzo)furyl, quinolyl, tetrahydroquinolyl, quinoxalyl or indolyl. The substituents on the heteroaryl group may be selected from the group of substituents listed for the aryl group. The heteroaryl group may be attached via a carbon atom or a heteroatom, if feasible.

The term “heterocyclic group” refers to radicals or groups derived from monocyclic or polycyclic saturated or unsaturated, substituted or unsubstituted heterocyclic nuclei having 5, 6, 7, 8, 9, 10, 11, 12, 13 or 14 ring atoms and containing 1, 2 to 3 hetero atoms selected from the group consisting of nitrogen, oxygen or sulfur.

The term substituent is “non-interfering” substituents. By “non-interfering” is meant that the group is suitable chemically and stability wise to occupy the designated position and perform the designated or intended role. Thus unsuitable groups are excluded from the definition of “non-interfering”.

In addition, compounds of Formula (I), (II), (III), Benozfuran lignan and derivatives thereof may be labeled with an isotope (e.g., ³H, ¹⁴C, ³⁵S, ¹²⁵I, etc.).

A “prodrug” refers to a compounds capable of being converted to compounds of the present invention by reactions of an enzyme, gastric juice, or the like, under physiological conditions in vivo, specifically compounds capable of being converted to compounds of the present invention upon enzymatic oxidation, reduction, hydrolysis, or the like, or a compounds capable of being converted to compounds of the present invention upon hydrolysis or the like by gastric juice or the like.

A “polymorph” refers to a compound that occurs in two or more forms.

The phrase “therapeutically effective amount” means an amount of a compound of the present invention that—treat or prevent the particular disease, condition, or disorder; or attenuates, ameliorates, or eliminates one or more symptoms of the particular disease, condition, or disorder; or prevents o delays the onset of one or more symptoms of the particular disease, condition, or disorder described herein.

As used herein, “treatment” or “treating” is an approach for obtaining beneficial or desired results including clinical results. For purposes of this invention, beneficial or desired clinical results include, but are not limited to, one or more of the following: decreasing one more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, increasing the quality of life, and/or prolonging survival (including overall survival and progression free survival. In some embodiments, the composition reduces the severity of one or more symptoms associated with cancer by at least about any of 10%, 20%, 30%, 40%, 50%, 60%, 70%, 80%, 90%, 95%, or 100% compared to the corresponding symptom in the same subject prior to treatment or compared to the corresponding symptom in other subjects not receiving the composition. Also encompassed by “treatment” is a reduction of pathological consequence of cancer. The methods of the invention contemplate any one or more of these aspects of treatment.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, such as routine physical exams or x-ray. Development may also refer to disease progression that may be initially undetectable and includes occurrence and onset.

“Adjuvant setting” refers to a clinical setting in which an individual has had a history of cancer, and generally (but not necessarily) been responsive to therapy, which includes, but is not limited to, surgery (e.g., surgical resection), radiotherapy, and chemotherapy. However, because of their history of the cancer, these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated.

“Neoadjuvant setting” refers to a clinical setting in which the method is be carried out before the primary/definitive therapy.

As used herein, an “at risk” individual is an individual who is at risk of developing cancer. An individual “at risk” may or may not have detectable disease, and may or may not have displayed detectable disease prior to the treatment methods described herein. “At risk” denotes that an individual has one or more so-called risk factors, which are measurable parameters that correlate with development of cancer, which are described herein. An individual having one or more of these risk factors has a higher probability of developing cancer than an individual without these risk factor(s).

As used herein, by “pharmaceutically active compound” is meant a chemical compound that induces a desired effect, e.g., treating, stabilizing, preventing, and/or delaying cancer.

As used herein, by “combination therapy” is meant a first therapy that includes compositions comprising novel compounds of the invention in conjunction with a second therapy (e.g., surgery or a chemotherapeutic agent) useful for treating, stabilizing, preventing, and/or delaying cancer. Administration in “conjunction with” another compound includes administration in the same or different composition(s), either sequentially, simultaneously, or continuously. In some embodiments, the combination therapy optionally includes one or more pharmaceutically acceptable carriers or excipients, non-pharmaceutically active compounds, and/or inert substances.

The term “effective amount” refers to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response or a complete response), increase overall survival time, and/or improve one or more symptoms of cancer (e.g. as assessed by FOSI).

As is understood in the art, an “effective amount” may be in one or more doses, i.e., a single dose or multiple doses may be required to achieve the desired treatment endpoint. An effective amount may be considered in the context of administering one or more therapeutic agents, and a nanoparticle composition comprising a compound of the invention may be considered to be given in an effective amount if, in conjunction with one or more other agents, a desirable or beneficial result may be or is achieved.

In some embodiments, the amount of the composition, first therapy, second therapy, or combination therapy is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

### COMPOUNDS OF THE PRESENT INVENTION

The present invention relates to the compounds of formula (I) and derivatives thereof including but not limited to polymorphs, isomers and prodrugs thereof, geometric or optical isomers thereof, and pharmaceutically acceptable esters, ethers, carbamates of such compounds, all solvates and hydrates thereof and all salts thereof. However, in accordance with the present invention the R group is preferably selected from vanillic acid, ferulic acid, eugenol, salicylic acid and/or their derivatives.

Particularly the present invention provides the following exemplary compounds of Formula I which are represented by structure numbers as follows:
Compound 1. Methyl 4-{[(2E)-3-(3,4-dihydroxyphenyl)prop-2-enoyl]oxy}-3-methoxy benzoate. (CAMVE)
Compound 2. 2-methoxy-4-[(1E)-3-methoxy-3-oxoprop-1-en-1-yl]phenyl (2E)-3-(3,4 dihydroxy phenyl)acrylate.
Compound 3. Methyl 2-{[(2E)-3-(3,4-dihydroxyphenyl)prop-2-enoyl]oxy}benzoate.
Compound 4. 4-Allyl-2-methoxyphenyl (2E)-3-(3,4-dihydroxyphenyl)acrylate.
Compound 5. (±)-2β-[4-O-(3,4,-dihydroxycinnamyl)-3-methoxyphenyl]-3α-methyl-7-methoxy-5-[(E)-1-propenyl]-2,3-dihydrobenzofuran
Compound 6. Methyl(E)-3-[2β-{4-O-(3,4-dihydroxycinnamyl)-3-methoxyphenyl}-7-methoxy-3α-methoxycarbonyl-2,3-dihydro-1-benzofuran-5-yl]propen-2-enate.
Compound 7. 2-Methoxy-4-[(1E)-prop-1-en-1-yl]phenyl (2E)-3-(3,4-dihydroxyphenyl)acrylate
Compound 8. 4-Formyl-2-methoxyphenyl (2E)-3-(3,4-dihydroxyphenyl)acrylate

The present invention further relates to the compounds of formula (II) and derivatives thereof including but not limited to polymorphs, isomers and prodrugs thereof, geometric or optical isomers thereof, and pharmaceutically acceptable esters, ethers, carbamates of such compounds, all solvates and hydrates thereof and all salts thereof. However in accordance with the present invention the R group is preferably selected from Vanillic acid, ferulic acid, eugenol, salicylic acid and/or their derivatives.

The present invention provides the following exemplary compounds of formula II:
Compound 9. 4-(Methoxycarbonyl)phenyl 4-hydroxy-3-methoxybenzoate
Compound 10. 2-Methoxy-4-(methoxycarbonyl)phenyl 4-hydroxy-3-methoxybenzoate
Compound 11. 2-Methoxy-4-[(1E)-3-methoxy-3-oxoprop-1en-1yl]phenyl 4hydroxy-3-methoxy benzoate.
Compound 12. Methyl(E)-3-[2β-{4-O-(3-methoxy-4-hydroxyphenyl carbonyl)-3-methoxyphenyl}-7-methoxy-3α-methoxycarbonyl-2,3-dihydro-1-benzofuran-5yl]prop-2-enoate
Compound 13. 4-Allyl-2-methoxyphenyl 4-hydroxy-3-methoxybenzoate.
Compound 14. 2-Methoxy-4-[(1E)-prop-1en-1-yl]phenyl 4-hydroxy-3-methoxybenzoate.
Compound 15. 4-Formyl-2-methoxyphenyl 4-hydroxy-3-methoxybenozoate.
Compound 16. (±)-2β-[4-O-(3-Hydroxy-4-methoxy)-3-methoxyphenyl]-3α-methyl-7-methoxy-5-[(E)-1-propenyl]-2,3-dihydrobenzofuran.

The present invention further relates to the compounds of formula (III) and derivatives thereof including but not limited to polymorphs, isomers and prodrugs thereof, geometric or optical isomers thereof, and pharmaceutically acceptable esters, ethers, carbamates of such compounds, all solvates and hydrates thereof and all salts thereof. However in accordance with the present invention the R group is preferably selected from Vanillic acid, ferulic acid, eugenol, salicylic acid and/or their derivatives.

The present invention provides the following exemplary compounds of Formula III:
Compound 17. Methyl 4-{[(2E)-3-(4-hydroxyphenyl)prop-2-enoyl]oxy}-3-methoxybenzoate
Compound 18. 2-methoxy-4-[(1E)-3-methoxy-3-oxoprop-1-en-1-yl]phenyl(2E)-3-(4-hydroxy phenyl)acrylate
Compound 19. 4-Formyl-2-methoxyphenyl (2E)-3-(4-hydroxyphenyl)acrylate
Compound 20. 2-Methoxyphenyl (2E)-3-(4-hydroxyphenyl)acrylate
Compound 21. 4-Allyl-2-methoxyphenyl (2E)-3-(4-hydroxyophenyl)acrylate
Compound 22. Methyl [3,4-bis O-(4-hydroxyphenylacryloyl)]phenylacrylate.
Compound 23. 2-Methoxy-4-(1E)-prop-1en-1yl]phenyl (2e)-3-(4-hydroxyphenyl)acrylate
Compound 24. Methyl (E)-3-[2β-{4-O-(4-hydroxycinnamoyl)-3-methoxyphenyl}-7-methoxy-3α-methoxycarbonyl-2,3-dihydro-1-benzofuran-5-yl-prop-2-enoate

Particularly the present invention provides the compounds of benzofuran derivatives
Compound 25. (±)-2β-{4-O-(3-methoxy-4-hydroxy cinnamoyl)-3-methoxyphenyl]-3α-methyl-7-methoxy-5-[(E)-1-propenyl]-2,3-dihydrobenzofuran.
Compound 26. 2-methoxy-4-(methoxycarbonyl)phenyl 3,4,5-trihydroxybenzoate
Compound 27. 5-[(E)-2-carboxyvinyl]-2β-(4-hydroxy-3-methoxyphenyl)-7-methoxy-2,3-dihydro-1-benzofuran-3α-carboxylic acid
Compound 28. 5-[(E)-2-carboxyvinyl]-7-hydroxy-2β-(4-hydroxy-3-methoxy phenyl)-2,3-dihydro-1-benzofuran-3α-carboxylic acid
Compound 29. (±)-2β-[4-O-(4-hydroxy cinnamoyl)-3-methoxyphenyl]-3α-methyl-7-methoxy-5-[(E)-1-propenyl]-2,3-dihydrobenzofuran.

Accordingly, the present invention also encompasses prodrugs of compounds of the present invention. Suitable active metabolites of compounds within the scope of Formulas (I), (II) (III), or benzofuran lignan derivatives in any suitable form, are also included herein.

The compounds of the present invention may contain asymmetric or chiral centers, and therefore may exist in different stereoisomeric forms. All suitable optical isomers and stereoisomeric forms of the compounds of the present invention as well as mixtures thereof, including racemic mixtures, form part of the present invention. In addition, the present invention embraces all geometric and positional isomers. Moreover, some compounds of the present invention may exhibit polymorphism. The present invention includes all polymorphic forms of the compounds according to the invention, which forms the further aspect of the invention. It is to be understood that the present invention encompasses any and all racemic, optically-active, polymorphic and stereoisomeric forms, or mixtures thereof, which form or forms possess properties useful in the treatment of the conditions indicated herein.

Furthermore, the present invention also include isotopically-labeled compounds of the present invention which are identical to those recited herein, but for the fact that one or more atoms are replaced by an atom having an atomic mass or mass number different from the atomic mass or mass number usually found in nature.

### Preparation of Compounds of the Present Invention

The present invention provides process for preparation of compounds of Formulas I, II, III. Those skilled in the art will understand from this disclosure how to prepare the most preferred compounds of the present invention using any suitable known method. Compounds of Formulas I, II, III and benzofuran lignan derivatives unless otherwise indicated, R, as described above may be conveniently prepared according to general process as given herein later.

In addition, the examples provided herein further illustrate the preparation of the compounds of the present invention. Moreover, those skilled in the art will understand from the present disclosure how to modify Scheme I, and the details of the examples described hereinafter to prepare any specific compound of Formulas I, II, III and benzofuran lignan derivatives of the present invention as desired. It should be understood that Scheme I is provided solely for the purposes of illustration and depicts potential route for synthesizing compounds of Formulas I, II, III and benzofuran lignan derivatives and does not limit the invention.

Those skilled in the art will appreciate that other synthetic routes may be used to synthesize the compounds of the present invention. Although specific starting materials and reagents are depicted in the examples illustrated below, the suitable substitution can be easily made to provide a variety of derivatives and reaction conditions. In addition, many of the compounds prepared by the method described below can be further modified in light of the disclosure using the conventional chemistry known to those skilled in the art.

### Formulations of Compounds of the Present Invention

A compound of Formulas (I), (II), (III) or benzofuran lignan derivatives or a derivative thereof can be administered in any conventional form not limited to oral, buccal, nasal, inhalation spray in unit dosage form, parenteral, (for example, intravenous, intramuscular, subcutaneous intrasternal or by infusion techniques), topical (for example, powder, ointment or drop), transdermal, intracisternal, intravaginal, intraperitoneal, intravesical, or rectal,. In another aspect of the invention, the compound of the present invention and at least one other pharmaceutically active agent may be administered either separately or in the pharmaceutical composition comprising both.

The compounds of this invention may also be administered to a mammal other than a human. The method of administration and the dosage to be administered to such a mammal will depend, for example, on the animal species and the disease or disorder being treated. The compounds of this invention may be administered to animals in any suitable manner, e.g., orally, parenterally or transdermally, in any suitable form such as, for example, a capsule, bolus, tablet, pellet, or pill. Such formulations are prepared in a conventional manner in accordance with standard veterinary practice. A solid dose formulation according to the invention is a solid gel (e.g. a gel which is flexible but which has dimensional stability), pastille, compressed tablet, lozenge, capsule etc, or a gel-spray. The dosage units are preferably homogeneous in composition, but also included within the scope of the invention are multi-layered dosage units formed from layers of differing composition, for example two-layered tablets and gels in which the different layers contain different active ingredients and/or exhibit different release characteristics.

The pharmaceutical formulation comprising a compound of Formulas (I), (II), (III) or benzofuran lignan derivatives or the derivatives thereof may be formulated in a conventional manner known to those skilled at the art using one or more pharmaceutically acceptable diluent, carrier, or vehicle.

In some embodiments, the compositions of the invention also include a stabilizing agent for use in the methods of treatment, methods of administration, and dosage regimes described herein. In some embodiments, the compositions of the invention include an antimicrobial agent and/or a sugar and/or a stabilizing agent for use in the methods of treatment, methods of administration, and dosage regimes described herein. The present invention in another variation provides for compositions and methods of preparation which retain the desirable therapeutic effects and remain physically and/or chemically stable upon exposure to certain conditions such as prolonged storage, elevated temperature, or dilution for parenteral administration. The stabilizing agent includes, for example, chelating agents (e.g., citrate, malic acid, edetate, or pentetate), sodium pyrophosphate, and sodium gluconate. In some embodiments, the invention provides pharmaceutical formulations of compositions comprising a compound of Formulas (I), (II), (III) or benzofuran lignan derivatives comprising sodium citrate, sodium pyrophosphate, EDTA, sodium gluconate, and/or sodium chloride. In another variation, the invention provides compositions comprising a compound of Formulas (I), (II), (III) or benzofuran lignan derivatives used for preparing the formulation in an anhydrous form prior to being incorporated into the formulation.

In some embodiments, a stabilizing agent is not contained or used in the methods of treatment, methods of administration, and dosage regimes described herein. In some embodiments a solubility enhancer such as polyoxyethylene castor oil derivatives, particularly cremophor is included.

Further excipients may be included in the formulations according to the invention as appropriate. For example, the formulations may include one or more antioxidants. Preferred antioxidants include alpha-tocopherol, ascorbyl palmitate, butylated hydroxy anisole (BHA) etc. The formulation may also include one or more coloring agents. Suitable coloring agents include, for example, curcumin or chlorophylls.

It is known that the delivery of biologics in the form of a particulate suspension allows targeting to organs such as the liver, lungs, spleen, lymphatic circulation, and the like, due to the uptake in these organs, of the particles by the reticuloendothelial (RES) system of cells. Targeting to the RES containing organs may be controlled through the use of particles of varying size, and through administration by different routes. Suitable nontoxic pharmaceutically acceptable excipients for use in the compositions of the present invention will be apparent to those skilled in the art of pharmaceutical formulations and examples are described in REMINGTON: The Science and Practice of Pharmacy, 20th Edition, A. R. Gennaro, ed., (2000). The choice of suitable carriers will depend on the exact nature of the particular dosage form desired, e.g., whether the compounds of the invention are to be formulated into microemulsions, suspensions, microparticles, or nanoparticles, as well as on the physicochemical properties of the compounds.

### Administration of Compounds of the Invention

The dose of a compound of Formulas (I), (II), (III) or benzofuran lignan derivatives or derivatives thereof to be administered to a mammal including human or animal for the purposes as mentioned above is not specifically limited. Rather it is widely variable and subject to the pathologies, conditions, symptoms, or age of the subject and judgment of the attending physician or veterinarian. While it may be practical to administer the daily dose of a compound of this invention, in portions, at various hours of the day, in any given case, the amount of compound of this invention will depend on such factors as the solubility of the compound, prodrug, isomer or pharmaceutically acceptable salt of this invention, the formulation used and the route of administration (e.g., orally, transdermally, parenterally or topically).

By the term “administering,” it is meant that compositions comprising a compound of Formulas (I), (II), (III) or benzofuran lignan derivatives are delivered to the host in such a manner that it can achieve the desired purpose. As mentioned The compositions can be administered by an effective route, such as orally, topically, rectally, etc. The compositions can be administered to any host in need of treatment, e.g., vertebrates, such as mammals, including humans, male humans, female humans, primates, pets, such as cats and dogs, livestock, such as cows, horses, birds, chickens, etc.

An “effective amount” of the compositions are administered to such a host. Effective amounts are such amounts which are useful to achieve the desired effect, preferably a beneficial or therapeutic effect as described above. Such amount can be determined routinely, e.g., by performing a dose-response experiment in which varying doses are administered to cells, tissues, animal models to determine an effective amount for achieving a desired result. Amounts are selected based on various factors, including the milieu to which the composition is administered (e.g., a cancer patient, animal model, tissue culture cells, etc.), the site of the cells to be treated, the age, health, gender, and weight of a patient or animal to be treated, etc. Useful amounts include, 10 milligrams-100 grams, preferably, e.g., 100 milligrams-10 grams, 250 milligrams-2.5 grams, 1 gm, 2 gm, 3 gm, 500 milligrams-1.25 grams. etc., per dosage of different forms of the compositions depending upon the need of the recipients and the method of preparation.

The term “effective amount” used herein further refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to cause a tumor to shrink and/or to decrease the growth rate of the tumor (such as to suppress tumor growth). In some embodiments, an effective amount is an amount sufficient to delay development. In some embodiments, an effective amount is an amount sufficient to prevent occurrence and/or recurrence. An effective amount can be administered in one or more administrations. The compositions described herein can be administered alone or in combination with other pharmaceutical agents, including poorly water soluble pharmaceutical agents. For example, a compounds of Formulas (I), (II), (III) or benzofuran lignan derivatives can be co-administered with one or more other chemotherapeutic agents including, but not limited to, carboplatin, Navelbine® (vinorelbine), anthracycline (Doxil), lapatinib (GW57016), Herceptin, gemcitabine (Gemzar®), capecitabine (Xeloda®), alimta, cisplatin, 5-fluorouracil, epirubicin, cyclophosphamide, avastin, velcade®, etc. In some embodiments, the compounds of Formulas (I), (II), (III) or benzofuran lignan derivatives are co-administered with a chemotherapeutic agent selected from the group consisting of antimetabolites (including nucleoside analogs), platinum-based agents, alkylating agents, tyrosine kinase inhibitors, anthracycline antibiotics, vinca alkloids, proteasome inhibitors, macrolides, and topoisomerase inhibitors. These other pharmaceutical agents can be present in the same composition as the compounds of Formulas (I), (II), (III) or benzofuran lignan derivatives are, or in a separate composition that is administered simultaneously or sequentially with the compositions comprising the compounds of Formulas (I), (II), (III) or benzofuran lignan derivatives.

The amount of the inventive composition administered to an individual (such as a human) may vary with the particular composition, the method of administration, and the particular type of recurrent cancer being treated. The amount should be sufficient to produce a desirable beneficial effect. For example, in some embodiments, the amount of the composition is effective to result in an objective response (such as a partial response or a complete response). In some embodiments, the amount of the compounds of Formulas (I), (II), (III) or benzofuran lignan derivatives is sufficient to result in a complete response in the individual. In some embodiments, the amount of the compounds of Formulas (I), (II), (III) or benzofuran lignan derivatives is sufficient to result in a partial response in the individual. In some embodiments, the amount of the taxane nanoparticle composition administered alone is sufficient to produce an overall response rate of more than about any of 40%, 50%, 60%, or 64% among a population of individuals treated with the taxane nanoparticle composition. Responses of an individual to the treatment of the methods described herein can be determined, for example, based on RECIST or CA-125 level. For example, when CA-125 is used, a complete response can be defined as a return to a normal range value of at least 28 days from the pretreatment value. A partial response can be defined as a sustained over 50% reduction from the pretreatment value.

In some embodiments, the amount of the composition is sufficient to prolong progress-free survival of the individual (for example as measured by RECIST or CA-125 changes). In some embodiments, the amount of the composition is sufficient to prolong overall survival of the individual. In some embodiments, the amount of the composition is sufficient to produce clinical benefit of more than about any of 50%, 60%, 70%, or 77% among a population of individuals treated with the taxane nanoparticle composition.

In some embodiments, the amount of the compound of Formulas (I), (II), (III) or benzofuran lignan derivatives in the composition is below the level that induces a toxicological effect (i.e., an effect above a clinically acceptable level of toxicity) or is at a level where a potential side effect can be controlled or tolerated when the composition is administered to the individual. In some embodiments, the amount of the composition is close to a maximum tolerated dose (MTD) of the composition following the same dosing regime. In some embodiments, the amount of the composition is more than about any of 80%, 90%, 95%, or 98% of the MTD.

In some embodiments, the amount of compounds of Formulas (I), (II), (III) or benzofuran lignan derivatives in the in the effective amount of the composition (e.g., a unit dosage form) is in the range of about 5 mg to about 500 mg, such as about 30 mg to about 300 mg or about 50 mg to about 200 mg. In some embodiments, the concentration of the compounds of Formulas (I), (II), (III) or benzofuran lignan derivatives in the composition is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml.

Exemplary effective amounts of compounds of Formulas (I), (II), (III) or benzofuran lignan derivatives in the composition is about 5 to about 300 mg/m² of a subject, such as about 100 to about 150 mg/m², about 120 mg/m², about 130 mg/m², or about 140 mg/m².

In some embodiments of any of the above aspects, the effective amount of compounds of Formulas (I), (II), (III) or benzofuran lignan derivatives in the composition includes at least about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of the subject.

Exemplary dosing frequencies include, but are not limited to, weekly without break; weekly, three out of four weeks; once every three weeks; once every two weeks; weekly, two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7×(i.e., daily) a week. In some embodiments, the intervals between each administration are less than about any of 6 months, 3 months, 1 month, 20 days, 15, days, 10 days, 7 days, 5 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

The administration of the composition can be extended over an extended period of time, such as from about a month up to about seven years. In some embodiments, the composition is administered over a period of at least about any of 2, 3, 4, 5, 6, 7, 8, 9, 10, 11, 12, 18, 24, 30, 36, 48, 60, 72, or 84 months. The dosing frequency of the composition may be adjusted over the course of the treatment based on the judgment of the administering physician.

The compositions described herein allow infusion of the composition to an individual under an infusion time that is shorter than about 24 hours. In some embodiments, the composition is administered over an infusion period of about 30 minutes or more.

In some embodiments, the invention provides a method of treating cancer in an individual by parenterally administering to the individual (e.g., a human) an effective amount of a composition comprising compounds of Formulas (I), (II), (III) or benzofuran lignan derivatives. The invention also provides a method of treating cancer in an individual by intravenous, intra-arterial, intramuscular, subcutaneous, inhalation, intraperitoneal, nasally, or intra-tracheal administering to the individual (e.g., a human) an effective amount of a composition comprising compounds of Formulas (I), (II), (III) or benzofuran lignan derivatives. In some embodiments, the route of administration is intraperitoneal. In some embodiments, the route of administration is intravenous, intra-arterial, intramuscular, or subcutaneous. In some embodiments, the compounds of Formulas (I), (II), (III) or benzofuran lignan derivative is the only pharmaceutically active agent for the treatment of cancer that is contained in the composition.

Any of the compositions described herein can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In one variation of the invention, nanoparticles (such as albumin nanoparticles) of the inventive compounds can be administered by any acceptable route including, but not limited to, orally, intramuscularly, transdermally, intravenously, through an inhaler or other air borne delivery systems and the like.

In some embodiments, compounds of Formulas (I), (II), (III) or benzofuran lignan derivatives may be administered with a second therapeutic compound and/or a second therapy. The dosing frequency of the compounds of Formulas (I), (II), (III) or benzofuran lignan derivatives and the second compound may be adjusted over the course of the treatment based on the judgment of the administering physician. In some embodiments, the first and second therapies are administered simultaneously, sequentially, or concurrently. When administered separately, the compounds of Formulas (I), (II), (III) or benzofuran lignan derivatives and the second compound can be administered at different dosing frequency or intervals. In some embodiments, sustained continuous release formulation of the compounds of Formulas (I), (II), (III) or benzofuran lignan derivatives and/or second compound may be used. Various formulations and devices for achieving sustained release are known in the art. A combination of the administration configurations described herein can be used.

The present invention also provides metronomic therapy regimes for any of the methods of treatment and methods of administration described herein. In some embodiments, the compound of Formulas (I), (II), (III) or benzofuran lignan derivatives is administered over a period of at least one month, wherein the interval between each administration is no more than about a week, and wherein the dose of the compound of Formulas (I), (II), (III) or benzofuran lignan derivatives at each administration is about 0.25% to about 25% of its maximum tolerated dose following a traditional dosing regime.

### Uses of the Compounds of the Invention

The present invention provides compounds of formula I, II, III and benzofuran lignan derivatives for the methods of treatment of diseases or conditions associated with NF kappa B modulation.

As indicated herein the compounds of the present inventions are useful more particularly in inflammatory conditions such as rheumatoid arthritis, inflammatory bowel disease, asthma, dermatosis including psoriasis, atopic dermatitis, and other conditions wherein NF kappa B modulation/activation is indicated.

The compounds are also useful in autoimmune diseases, tissue and organ rejections in transplantations, Alzheimer's diseases, stroke, atherosclerosis, restenosis.

The compounds of the present invention are also useful in cancer where NF kappa B transcription factor is involved. Cancers to be treated by compositions comprising compounds of formula I, II, III and benzofuran lignan derivatives include, but are not limited to, carcinoma, lymphoma, blastoma, sarcoma, and leukemia. Examples of cancers that can be treated by compositions described herein include, but are not limited to, squamous cell cancer, lung cancer (including small cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, melanoma, endometrical or uterine carcinoma, salivary gland carcinoma, kidney or renal cancer, liver cancer, prostate cancer, vulval cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome. In some embodiments, there is provided a method of treating metastatic cancer (that is, cancer that has metastasized from the primary tumor). In some embodiments, there is provided a method of reducing cell proliferation and/or cell migration. In some embodiments, there is provided a method of treating hyperplasia. In one aspect, compounds of formula I, II, III and benzofuran lignan derivatives for treating breast, ovary, testicle, prostate, head, neck, eye, skin, mouth, throat, esophagus, chest, bone, lung, colon, sigmoid, rectum, stomach, kidney, liver, pancreas, brain, intestine, heart or adrenal cancer or neoplastic disease are provided.

In some embodiments, formulations of compounds of formula I, II, III and benzofuran lignan derivatives for treating cancer at advanced stage(s) are provided. In some embodiments, there are provided methods of treating breast cancer (which may be HER2 positive or HER2 negative), including, for example, advanced breast cancer, stage IV breast cancer, locally advanced breast cancer, and metastatic breast cancer. In some embodiments, the cancer is lung cancer, including, for example, non-small cell lung cancer (NSCLC, such as advanced NSCLC), small cell lung cancer (SCLC, such as advanced SCLC), and advanced solid tumor malignancy in the lung. In some embodiments, the cancer is ovarian cancer, head and neck cancer, gastric malignancies, melanoma (including metastatic melanoma), colorectal cancer, pancreatic cancer, and solid tumors (such as advanced solid tumors). In some embodiments, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, gliomas, glioblastomas, neuroblastomas, and multiple myeloma. In some embodiments, the cancer is a solid tumor. In some embodiments, the cancer is selected from the group consisting of prostate cancer, colon cancer, breast cancer, head and neck cancer, pancreatic cancer, lung cancer, and ovarian cancer.

The compounds of the present invention are also useful in certain viral infections such as AIDS, osteoarthritis, osteoporosis.

Not limited to the above said conditions and disorders wherein NF kappa B is modulated, the present invention provides compounds which are useful in other inflammatory and cancer conditions.

The compounds of the present invention are also useful in combination therapies either given along with other medications or therapies.

Suitable anti-proliferative drugs or cytostatic compounds to be used in combination with the agents of the invention include anti-cancer drugs. Anti-cancer drugs are well known and include: Acivicin®; Aclarubicin®; Acodazole Hydrochloride®; Acronine®; Adozelesin®; Aldesleukin®; Altretamine®; Ambomycin®; Ametantrone Acetate®; Aminoglutethimide®; Amsacrine®; Anastrozole®; Anthramycin®; Asparaginase®; Asperlin®; Azacitidine®; Azetepa®; Azotomycin®; Batimastat®; Benzodepa®; Bicalutamide®; Bisantrene Hydrochloride®; Bisnafide Dimesylate®; Bizelesin®; Bleomycin Sulfate®; Brequinar Sodium®; Bropirimine®; Busulfan®; Cactinomycin®; Calusterone®; Caracemide®; Carbetimer®; Carboplatin®; Carmustine®; Carubicin Hydrochloride®; Carzelesin®; Cedefingol®; Chlorambucil®; Cirolemycin®; Cisplatin®; Cladribine®; Crisnatol Mesylate®; Cyclophosphamide®; Cytarabine®; Dacarbazine®; Dactinomycin®; Daunorubicin Hydrochloride®; Decitabine®; Dexormaplatin®; Dezaguanin®; Dezaguanine Mesylate®; Diaziquone®; Docetaxel®; Doxorubicin®; Doxorubicin Hydrochloride®; Droloxifene®; Droloxifene Citrate®; Dromostanolone Propionate®; Duazomycin®; Edatrexate®; Eflornithine Hydrochloride®; Elsamitrucin®; Enloplatin®; Enpromate®; Epipropidine®; Epirubicin Hydrochloride®; Erbulozole®; Esorubicin Hydrochloride®; Estramustine®; Estramustine Phosphate Sodium®; Etanidazole®; Etoposide®; Etoposide Phosphate®; Etoprine®; Fadrozole Hydrochloride®; Fazarabine®; Fenretinide®; Floxuridine®; Fludarabine Phosphate®; Fluorouracil®; Fluorocitabine®; Fosquidone®; Fostriecin Sodium®; Gemcitabine®; Gemcitabine Hydrochloride®; Hydroxyurea®; Idarubicin Hydrochloride®; Ifosfamide®; Ilmofosine®; Interferon Alfa-2a®; Interferon Alfa-2b®; Interferon Alfa-n1®; Interferon Alfa-n3®; Interferon Beta-I a®; Interferon Gamma-I b®; Iproplatin®; Irinotecan Hydrochloride®; Lanreotide Acetate®; Letrozole®; Leuprolide Acetate®; Liarozole Hydrochloride®; Lometrexol Sodium®; Lomustine®; Losoxantrone Hydrochloride®; Masoprocol®; Maytansine®; Mechlorethamine Hydrochloride®; Megestrol Acetate®; Melengestrol Acetate®; Meiphalan®; Menogaril®; Mercaptopurine®; Methotrexate®; Methotrexate Sodium®; Metoprine®; Meturedepa®; Mitindomide®; Mitocarcin®; Mitocromin®; Mitogillin®; Mitomalcin®; Mitomycin®; Mitosper®; Mitotane®; Mitoxantrone Hydrochloride®; Mycophenolic Acid®; Nocodazole®; Nogalamycin®; Ormaplatin®; Oxisuran®; Paclitaxel®; Pegaspargase®; Peliomycin®; Pentamustine®; Peplomycin Sulfate®; Perfosfamide®; Pipobroman®; Piposulfan®; Piroxantrone Hydrochloride®; Plicamycin®; Plomestane®; Porfimer Sodium®; Porfiromycin®; Prednimustine®; Procarbazine Hydrochloride®; Puromycin®; Puromycin Hydrochloride®; Pyrazofurin®; Riboprine®; Rogletimide®; Safingol®; Safingol Hydrochloride®; Semustine®; Simtrazene®; Sparfosate Sodium®; Sparsomycin®; Spirogermanium Hydrochloride®; Spiromustine®; Spiroplatin®; Streptonigrin®; Streptozocin®; Sulofenur®; Talisomycin®; Taxol®; Taxotere®; Tecogalan Sodium®; Tegafur®; Teloxantrone Hydrochloride®; Temoporfin®; Teniposide®; Teroxirone®; Testolactone®; Thiamiprine®; Thioguanine®; Thiotepa®; Tiazoftirin®; Tirapazamine®; Topotecan Hydrochloride®; Toremifene Citrate®; Trestolone Acetate®; Triciribine Phosphate®; Trimetrexate®; Trimetrexate Glucuronate®; Triptorelin®; Tubulozole Hydrochloride®; Uracil Mustard®; Uredepa®; Vapreotide®; Verteporfin®; Vinblastine Sulfate®; Vincristine Sulfate®; Vindesine®; Vindesine Sulfate®; Vinepidine Sulfate®; Vinglycinate Sulfate®; Vinleurosine Sulfate®; Vinorelbine Tartrate®; Vinrosidine Sulfate®; Vinzolidine Sulfate®; Vorozole®; Zeniplatin®; Zinostatin®; Zorubicin Hydrochloride®.

Other anti-cancer drugs suitable for combination therapy include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminol evulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; aza osine; baccatin III derivatives; balanol; batimastat; BCR/ABL antagonists; benzochlorins; benzoylstaurosporine; beta-lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti cancer compound; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O₆-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

### EXAMPLES

The invention will now be illustrated with the aid of following non-limiting examples. It should be understood, however, that the invention is not limited to the solely to the particular examples given below. It will be apparent that those skill in the art that any modifications, both to the materials and methods, may be practiced without departing from the purpose and interest of this invention.

### General Processes for the Preparation of Compounds of Formula I, II and III

The following standard protocols were followed in the descriptions of the manufacture of the compounds:
a) All operations which were carried out at room temperature or ambient temperature were in the range of 18 to 25° C.
b) Evaporation of the solvent was carried out under reduced pressure (600-4000 pascals; 4.5-30 mm Hg) with a bath temperature of up to 40° C.

c) The course of the reaction was monitored by thin layer chromatography (TLC) and reaction times are given for illustration only.
d) Melting points are uncorrected, the melting points are given for the materials prepared as described, polymorphism may result in isolation of materials with different melting points in some preparations.
e) The structure and purity of all final products were assured by at least one of the following techniques: TLC, NMR (nuclear magnetic resonance) spectroscopy, IR (Infrared spectroscopy), or microanalytical data. and HPLC
f) Yields are given for illustration only.
g) When given, NMR data is in the form of delta (.delta.) values for major diagnostic protons given in parts per million (ppm) relative to tetramethylsilane (TMS) as internal standard determined at 300 MHz or 400 MHz using the indicated solvent.
h) chemical symbols have their usual meanings; the following abbreviations have also been used: v (volume), w (weight), B. P. (boiling point), M. R. (Melting range), M. pt. (melting point), L (liters), ml (milliliters), gms (grams), mg (milligrams), mol (moles), mmol (millimoles) eq (equivalents) deg C (degree centigrade), conc. HCl (concentrated hydrochloric acid) any other

### General Procedure for the Preparation of Compounds of Formula I, II and III

The starting material was the appropriate acid. Specifically, cinnamic acid for compounds of formula I, vanillic acid for the formula II and 3,4,-dihydroxy cinnamic acid for formula III.

The process involves esterification, protection of all hydroxyl groups as MOM ether followed by hydrolysis to get corresponding acid which reacts with phenolic compound to get corresponding fused ester derivative. The deprotection of hydroxyl groups using methanolic HCl yields the compound of invention, which is then purified by silica gel column chromatography and characterized by conventional techniques (¹H NMR, MASS). The resulting pure compound was then analysed for its melting point, NMR, CMR, Mass Spectroscopy to determine its final structure and purity.

### Example 1

### Methyl 4-{[(2E)-3-(3,4-dihydroxyphenyl)prop-2-enoyl]oxy}-3-methoxybenzoate

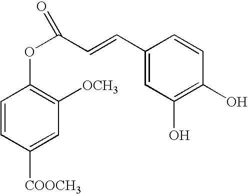

The above compound was prepared as per the general procedure by condensation of 3,4-dihydroxy cinnamic acid with methyl vannilate.

¹H NMR (CDCl₃, 400 MHz) δₚₚₘ: −3.8 (s, 3H, 1×Ar—OCH₃), 3.85 (s, 3H, 1×Ar—OCH₃), 6.42 (d, 1H, J=16 Hz), 6.8-7.7 (m, Ar—H), 7.8 (d, 1H, J=16 Hz), 8.05 (brs, 1H, —OH), 8.35 (brs, 1H, —OH). TOF MS ES: −367 (M⁺+Na). Molecular formula: —C₁₈H₁₆O₇. M. R.: 186-189° C.

### Example 2

### 2-methoxy-4-[(1E)-3-methoxy-3-oxoprop-1-en-1-yl]phenyl (2E)-3-(3,4 dihydroxy phenyl)acrylate

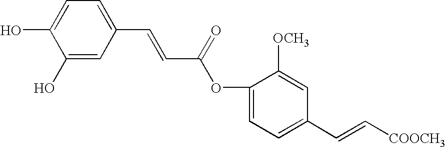

The above compound was prepared as per the general procedure by condensation of 3,4-dihydroxycinnamic acid with methyl ferulate.

¹H NMR (CDCl₃, 400 MHz) δₚₚₘ: −3.89 (s, 3H, 1×Ar—OCH₃), 3.92 (s, 3H, 1×Ar—OCH₃), 6.42 (d, 1H, J=15.7 Hz), 6.86-7.69 (m, Ar—H), 7.74 (d, 1H, J=15.7 Hz), 8.5 (broad hump, 2H, 2×—OH). TOF MS ES: −393 (M⁺+Na). Molecular formula: —C₂₀H₁₈O₇. M.R.: −182-188° C.

### Example 3

### Methyl 2-{[(2E)-3-(3,4-dihydroxyphenyl)prop-2-enoyl]oxy}benzoate

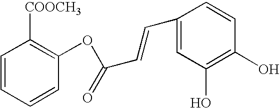

The above compound was prepared as per the general procedure by condensation of 3,4-dihydroxycinnamic acid with methyl salicylate.

¹H NMR (CDCl₃, 400 MHz) δₚₚₘ: −3.82 (s, 3H, 1×Ar—COOCH₃), 6.44 (d, 1H, J=15.8 Hz), 6.8-8.2 (m, Ar—H), 7.73 (d, 1H, J=15.8 Hz), 8.5 (brs, 1H, —OH), 8.8 (brs, 1H, —OH). TOF MS ES: −337 (M⁺+Na). Molecular formula: —C₁₇H₁₄O₆. M. R.: −152-154° C.

### Example 4

### 4-allyl-2-methoxyphenyl (2E)-3-(3,4-dihydroxyphenyl)acrylate

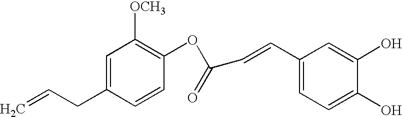

The above compound was prepared as per the general procedure by condensation of 3,4-dihydroxycinnamic acid with eugenol.

¹H NMR (DMSO-d₆, 400 MHz) δₚₚₘ: −3.74 (s, 3H, 1×Ar—OCH₃), 5.0-5.2 (m, 2H, Benzylic —CH₂), 5.9-6.1 (m, 1H, olefinic proton), 6.47 (d, 1H, J=15.8 Hz), 6.7-7.2 (m, Ar—H), 7.63 (d, 1H, J=15.8 Hz), TOF MS ES: −349 (M⁺+Na). Molecular formula: —C₁₉H₁₈O₅. M. R.: −139-142° C.

### Example 5

### (±)-2β-[4-O-(3,4,-dihydroxycinnamoyl)-3-methoxyphenyl]-3α-methyl-7-methoxy-5-[(E)-1-propenyl]-2,3 dihydrobenzofuran

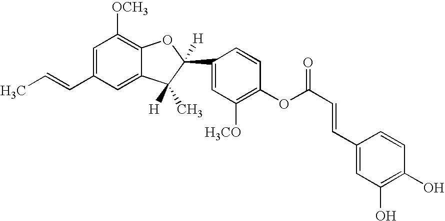

The above compound was prepared as per the general procedure by condensation of 3,4-dihydroxycinnamic acid with dehydrodiisoeugenol.

¹H NMR (DMSO-d₆, 400 MHz) δₚₚₘ: −1.38 (d, 3H, J=6.7 Hz), 1.82 (d, 3H, J=6.7 Hz), 3.77 (s, 3H, 1×Ar—OCH₃), 3.80 (s, 3H, 1×Ar—OCH₃), 5.21 (d, 1H, J=10 Hz), 6.0-6.2 (m, 1H, olefinic proton), 6.34 (d, 1H, J=15.8 Hz), 6.49 (d, 1H, J=15.8 Hz), 6.6-7.4 (m, Ar—H), 7.65 (d, 1H, J=15.8 Hz). TOF MS ES: −489 (M+H). Molecular formula: —C₂₉H₂₈O₇. M. R.: −94-98° C.

### Example 6

### Methyl (E)-3-[2β-{4-O-(3,4-dihydroxy cinnamoyl)-3-methoxyphenyl}-7-methoxy-3α-methoxycarbonyl-2,3-dihydro-1-benzofuran-5-yl]prop-2-enoate

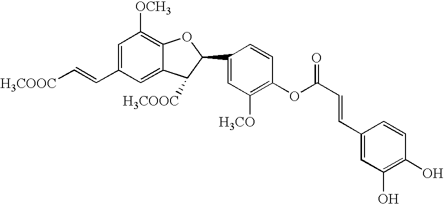

The above compound was prepared as per the general procedure by condensation of 3,4-dihydroxycinnamic acid with Methyl(E)-3-[2-(4-hydroxy-3-methoxyphenyl)-7-methoxy-3-methoxycarbonyl-2,3-dihydro-1-benzofuran-5-yl]prop-2-enoate.

¹H NMR (DMSO-d₆, 400 MHz) δₚₚₘ: −3.71 (s, 6H, 1×Ar—OCH₃), 3.76 (s, 6H, 2×Ar—COOCH₃), 3.86 (s, 3H, 1×Ar—OCH₃), 4.62 (d, 1H, J=7.3 Hz), 6.06 (d, 1H, J=7.9 Hz Benzylic proton), 6.4-7.6 (m, Ar—H and olefinic protons), 7.63 (d, 1H, J=15.9 Hz), 7.65 (d, 1H, J=15.9 Hz). TOF MS ES: −599 (M⁺+Na). Molecular formula: —C₃₁H₂₈O₁₁. M. R.: −176-180° C.

### Example 7

### 2-methoxy-4-[(1E)-prop-1-en-1-yl]phenyl(2E)-3-(3,4-dihydroxy phenyl)acrylate

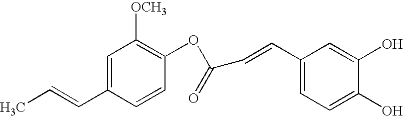

The above compound was prepared as per the general procedure by condensation of 3,4-dihydroxycinnamic acid with isoeugenol.

¹H NMR (DMSO-d₆, 400 MHz) δₚₚₘ: −1.85 (d, 1H, J=6.8 Hz), 3.78 (s, 3H, 1×Ar—OCH₃), 5.57 (d, 1H, J=7.8 Hz), 6.6-7.4 (m, Ar—H,), 7.63 (d, 1H, J=15.8 Hz), 9.2 (brs, 1H, —OH), 9.7 (brs, 1H, —OH). TOF MS ES: −349 (M⁺+Na). Molecular formula: —C₁₉H₁₈O₅. M. R.: −168-172° C.

### Example 8

### 4-formyl-2-methoxyphenyl (2E)-3-(3,4-dihydroxyphenyl)acrylate

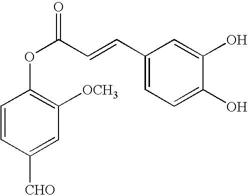

The above compound was prepared as per the general procedure by condensation of 3,4-dihydroxycinnamic acid with Vanillin.

¹H NMR (DMSO-d₆, 400 MHz) δₚₚₘ: −3.86 (s, 3H, 1×Ar—OCH₃), 6.52 (d, 1H, J=15.8 Hz), 6.7-7.5 (m, Ar—H), 7.69 (d, 1H, J=15.8 Hz), 9.6 (broad hump, 2H, 2×—OH), 9.99 (s, 1H, —CHO). TOF MS ES: −337 (M⁺+Na). Molecular formula: —C₁₇H₁₄O₆. M. R.: −154-159° C.

### Example 9

### 2-(Methoxycarbonyl)phenyl 4-hydroxy-3-methoxybenzoate

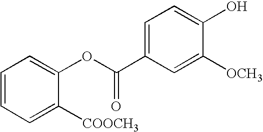

The above compound was prepared as per the general procedure by condensation of vanillic acid with methyl salicylate.

¹H NMR (CDCl₃, 500 MHz) δₚₚₘ: −3.66 (s, 3H, 1×Ar—OCH₃), 3.87 (s, 3H, 1×Ar—OCH₃), 6.1 (brs, 1H, —OH), 6.9-8.0 (m, Ar—H). TOF MS ES: −325 (M⁺+Na). Molecular formula: —C₁₆H₁₄O₆. M. R.: −86-88° C.

### Example 10

### 2-methoxy-4-(methoxycarbonyl)phenyl 4-hydroxy-3-methoxy benzoate

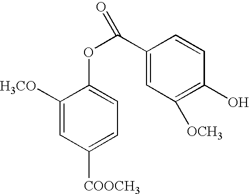

The above compound was prepared as per the general procedure by condensation of vanillic acid with methyl vanillate.

¹H NMR (CDCl₃, 300 MHz) δₚₚₘ: −3.87 (s, 3H, 1×Ar—OCH₃), 3.92 (s, 3H, 1×Ar—OCH₃), 3.97 (s, 3H, 1×Ar—OCH₃), 6.8-8.0 (m, Ar—H and —OH). TOF MS ES: −355 (M⁺+Na). Molecular formula: —C₁₇H₁₆O₇. M. R.: −131-133° C.

### Example 11

### 2-methoxy-4-[(1E)-3-methoxy-3-oxoprop-1-en-1yl]phenyl 4-hydroxy-3-methoxybenzoate

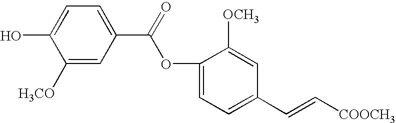

The above compound was prepared as per the general procedure by esterification of vanillic acid with methyl ferulate.

¹H NMR (CDCl₃, 400 MHz) δₚₚₘ: −3.81 (s, 3H, 1×Ar—OCH₃), 3.85 (s, 3H, 1×Ar—OCH₃), 3.96 (s, 3H, 1×Ar—OCH₃), 6.42 (d, 1H, J=16.2 Hz), 6.8-7.8 (m, Ar—H), 7.68 (d, 1H, J=16.5 Hz), 8.2 (brs, 1H, —OH). Molecular formula: —C₁₉H₁₈O₇. TOF MS ES: −381 (M⁺+Na). M. R.: −152-154° C.

### Example 12

### Methyl(E)-3-[2β-{4-O-(3-methoxy-4-hydroxyphenyl carbonyl)-3-methoxyphenyl}-7-methoxy-3α-methoxycarbonyl-2,3-dihydro-1-benzofuran-5-yl]prop-2-enoate

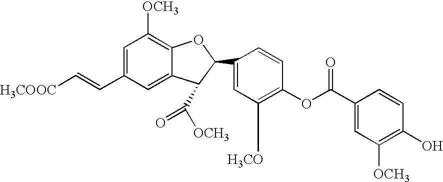

The above compound was prepared as per the general procedure by condensation of vanillic acid with Methyl (E)-3-[2β-(4-hydroxy-3-methoxyphenyl)-7-methoxy-3α-methoxycarbonyl-2,3-dihydro-1-benzofuran-5-yl]prop-2-enoate.

¹H NMR (CDCl₃, 400 MHz) δₚₚₘ: −3.81 (s, 6H, 1×Ar—COOCH₃), 3.86 (s, 3H, 1×Ar—OCH₃), 3.94 (s, 3H, 1×Ar—OCH₃), 3.96 (s, 3H, 1×Ar—OCH₃), 4.38 (d, 1H, J=8.4 Hz), 6.21 (d, 1H, J=7.7 Hz), 6.33 (d, 1H, J=16 Hz), 6.8-7.8 (m, Ar—H), 7.65 (d, 1H, J=16 Hz), 7.9 (brs, 1H, —OH). TOF MS ES: −587 (M⁺+Na). Molecular formula: —C₃₀H₂₈O₁₁. M. R.: −217-220° C.

### Example 13

### 4-allyl-2-methoxyphenyl 4-hydroxy-3-methoxybenzoate

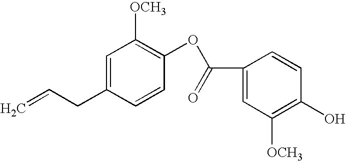

The above compound was prepared as per the general procedure by esterification of vanillic acid with eugenol.

¹H NMR (DMSO-d₆, 400 MHz) δₚₚₘ: −3.73 (s, 3H, 1×Ar—OCH₃), 3.84 (s, 3H, 1×Ar—OCH₃), 5.06-5.15 (m, 2H, Benzylic —CH₂), 5.94-6.03 (m, 1H, olefinic proton), 6.79-7.63 (m, Ar—H and olefinic protons), 10.17 (brs, 1H, —OH). TOF MS ES: −337 (M⁺+Na). Molecular formula: —C₁₈H₁₈O₅. M. R.: −68-71° C.

### Example 14

### 2-Methoxy-4-[(1E)-prop-1en-1-yl]phenyl4-hydroxy-3-methoxy benzoate

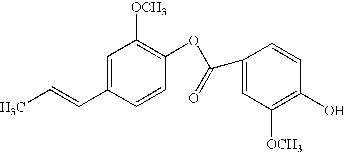

The above compound was prepared as per the general procedure by condensation of vanillic acid with isoeugenol.

¹H NMR (CDCl₃, 400 MHz) δₚₚₘ: −1.81 (d, 1H, J=8.0 Hz), 3.74 (s, 3H, 1×Ar—OCH₃), 3.87 (s, 3H, 1×Ar—OCH₃), 6.0-6.2 (m, 1H, olefinic proton), 6.31 (d, 1H, J=16 Hz), 6.8-7.8 (m, Ar—H). TOF MS ES: −337 (M⁺+Na). Molecular formula: —C₁₈H₁₈O₅. M. R.: 148-150° C.

### Example 15

### 4-formyl-2-methoxyphenyl 4-hydroxy-3-methoxybenozoate

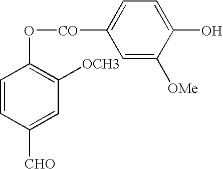

The above compound was prepared as per the general procedure by condensation of vanillic acid with vanillin.

¹H NMR (DMSO-d₆, 400 MHz) δₚₚₘ: −3.84 (s, 6H, 2×Ar—OCH₃), 6.8-7.8 (m, Ar—H), 9.99 (s, 1H, —CHO), 10.25 (s, 1H, —OH). TOF MS ES: −325 (M⁺+Na). Molecular formula: —C₁₆H₁₄O₆. M. R.: −134-138° C.

### Example 16

### (±)-2β-[4-O-(3-methoxy-4-hydroxy benzoyl)-3-methoxy phenyl]-3α-methyl-7-methoxy-5-[(E)-1-propenyl]-2,3-dihydrobenzofuran

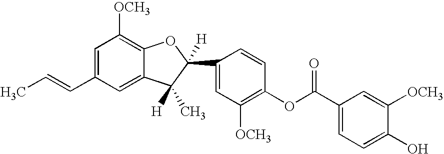

The above compound was prepared as per the general procedure by condensation of vanillic acid with dehydrodiisoeugenol.

¹H NMR (DMSO-d₆, 400 MHz) δₚₚₘ: −1.36 (d, 3H, J=6.7 Hz), 1.8 (d, 3H, J=5.5 Hz), 3.76 (s, 3H, 1×Ar—OCH₃), 3.80 (s, 3H, 1×Ar—OCH₃), 3.84 (s, 3H, 1×Ar—OCH₃), 4.0 (m, 1H), 5.23 (d, 1H, J=8.54 Hz), 6.0-6.2 (m, 1H, olefinic proton), 6.35 (d, 1H, J=15.8 Hz), 6.6-7.8 (m, Ar—H), 10.18 (s, 1H, —OH). TOF MS ES: −477 (M+H). Molecular formula: —C₂₈H₂₈O₇. M. R.: −136-140° C.

### Example 17

### methyl 4-{[(2E)-3-(4-hydroxyphenyl)prop-2-enoyl]oxy}-3-methoxy benzoate

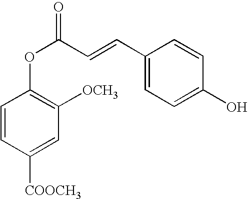

The above compound was prepared as per the general procedure by condensation of 4-hydroxycinnamic acid with methyl vanillate.

¹H NMR (CDCl₃, 400 MHz) δₚₚₘ: −3.89 (s, 3H, 1×Ar—COOCH₃), 3.93 (s, 3H, 1×Ar—OCH₃), 6.27 (brs, 1H, —OH). 6.59 (d, 1H, J=15.2 Hz), 6.8-7.8 (m, Ar—H), 7.83 (d, 1H, J=15.8 Hz). TOF MS ES: −351 (M⁺+Na). Molecular formula: —C₁₈H₁₆O₆. M. R.: −162-165° C.

### Example 18

### 2-methoxy-4-[(1E)-3-methoxy-3-oxoprop-1-en-1-yl]phenyl (2E)-3-(4-hydroxyphenyl)acrylate

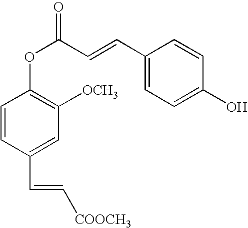

The above compound was prepared as per the general procedure by condensation of 4-hydroxy cinnamic acid with methyl ferulate.

¹H NMR (CDCl₃, 400 MHz) δₚₚₘ: −3.69 (s, 3H, 1×Ar—OCH₃), 3.75 (s, 3H, 1×Ar—COOCH₃), 6.29 (d, 1H, J=15.8 Hz), 6.34 (d, 1H, J=15.8 Hz), 6.6-7.4 (m, Ar—H), 7.54 (d, 1H, J=15.8 Hz), 7.67 (d, 1H, J=15.8 Hz), 9.37 (brs, 1H, —OH). TOF MS ES: −377 (M⁺+Na). Molecular formula: —C₂₀H₁₈O₆. M. R.: −174-180° C.

### Example 19

### 4-formyl-2-methoxyphenyl (2E)-3-(4-hydroxyphenyl)acrylate

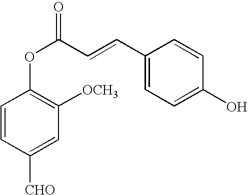

The above compound was prepared as per the general procedure by condensation of 4-hydroxy cinnamic acid with vanillin.

¹H NMR (DMSO-d₆, 400 MHz) δₚₚₘ: −3.86 (s, 3H, 1×Ar—OCH₃), 6.65 (d, 1H, J=16 Hz), 6.7-7.7 (m, Ar—H), 7.77 (d, 1H, J=16 Hz), 10.16 (s, 1H, —CHO), 10.27 (s, 1H, —OH). LCMS (Negative Mode, Q1MS): −297 (M−H). Molecular formula: —C₁₇H₁₄O₅. M. R.: 136-138° C.

### Example 20

### methyl 2-{[(2E)-3-(4-hydroxyphenyl)prop-2-enoyl]oxy}benzoate

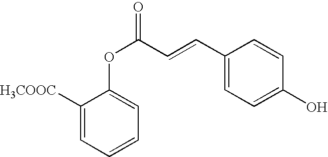

The above compound was prepared as per the general procedure by condensation of 4-hydroxy cinnamic acid with methyl salicylate.

¹H NMR (DMSO-d₆, 400 MHz) δₚₚₘ: −3.74 (s, 3H, 1×Ar—COOCH₃), 6.65 (d, 1H, J=15.8 Hz), 6.8-8.0 (m, Ar—H), 7.75 (d, 1H, J=15.8 Hz), 10.15 (s, 1H, —OH). TOF MS ES: −321 (M⁺+Na). Molecular formula: —C₁₇H₁₄O₅. M. R.: −160-163° C.

### Example 21

### 4-allyl-2-methoxyphenyl (2E)-3-(4-hydroxyophenyl)acrylate

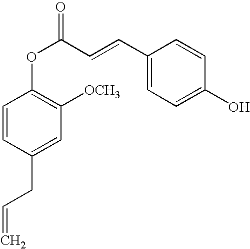

The above compound was prepared as per the general procedure by condensation of 4-hydroxy cinnamic acid with eugenol.

¹H NMR (DMSO-d₆, 400 MHz) δₚₚₘ: −3.74 (s, 3H, 1×Ar—OCH₃), 5.0-5.15 (m, 2H, Benzylic —CH₂), 5.8-6.1 (m, 1H, olefinic proton), 6.61 (d, 1H, J=15.8 Hz), 6.7-7.7 (m, Ar—H), 7.71 (d, 1H, J=15.8 Hz), 10.12 (s, 1H, —OH). TOF MS ES: −333 (M⁺+Na). Molecular formula: —C₁₉H₁₈O₄. M. R.: −147-151° C.

### Example 22

### methyl [3,4-bis O-(4-hydroxyphenylacryloyl)]phenylacrylate

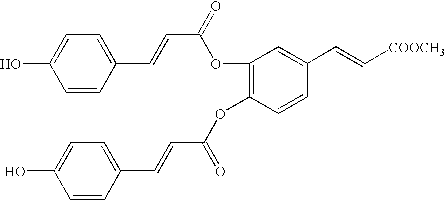

The above compound was prepared as per the general procedure by condensation of 4-hydroxy cinnamic acid with 3,4-dihydroxy methyl cinnamate.

¹H NMR (DMSO-d₆, 400 MHz) δₚₚₘ: −3.74 (s, 3H, 1×Ar—COOCH₃), 6.4-8.0 (m, Ar—H and olefinic protons), 10.14 (brs, 2H, 2×—OH). TOF MS ES: −513 (M⁺+Na). Molecular formula: —C₂₈H₂₂O₈. M. R.: −195-200° C.

### Example 23

### 2-methoxy-4-[(1E)-prop-1-en-1-yl]phenyl (2E)-3-(4-hydroxyphenyl)acrylate

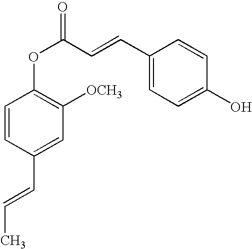

The above compound was prepared as per the general procedure by condensation of 4-hydroxy cinnamic acid with isoeugenol.

¹H NMR (DMSO-d₆, 400 MHz) δₚₚₘ: −1.85 (d, 1H, J=6.1 Hz), 3.78 (s, 3H, 1×Ar—OCH₃), 5.57 (d, 1H, J=7.8 Hz), 6.2-6.5 (m, olefinic proton), 6.61 (d, 1H, J=16.4 Hz), 6.7-7.7 (m, Ar—H), 7.72 (d, 1H, J=15.8 Hz), 10.12 (brs, 1H, —OH). TOF MS ES: −333 (M⁺+Na). Molecular formula: —C₁₉H₁₈O₄. M. R.: −195-200° C.

### Example 24

### methyl (E)-34213-{4-O-(4-hydroxy cinnamoyl)-3-methoxyphenyl}-7-methoxy-3α-methoxycarbonyl-2,3-dihydro-1-benzofuran-5-yl]prop-2-enoate

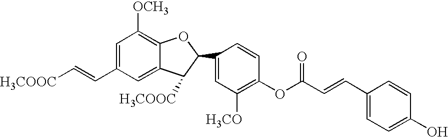

The above compound was prepared as per the general procedure by condensation of 4-hydroxy cinnamic acid with Methyl (E)-3-[2β-(4-hydroxy-3-methoxyphenyl)-7-methoxy-3α-methoxycarbonyl-2,3-dihydro-1-benzofuran-5-yl]prop-2-enoate.

¹H NMR (CDCl₃, 400 MHz) δₚₚₘ: −3.80 (s, 3H, 1×Ar—OCH₃), 3.83 (s, 3H, 2×Ar—OCH₃), 3.86 (s, 3H, 1×Ar—OCH₃), 3.94 (s, 3H, 1×Ar—OCH₃), 4.38 (d, 1H, J=7.9 Hz), 6.18 (d, 1H, J=7.9 Hz), 6.34 (d, 1H, J=15.8 Hz), 6.46 (d, 1H, J=15.8 Hz), 6.6-7.6 (m, Ar—H), 7.64 (d, 1H, J=15.8 Hz), 7.78 (d, 1H, J=15.9 Hz), 9.48 (brs, 1H, —OH). TOF MS ES: −583 (M⁺+Na). Molecular formula: —C₃₁H₂₈O₁₀. M. R.: −205-210° C.

### Example 25

### (±)-2β-[4-O-(3-methoxy-4-hydroxy cinnamoyl)-3-methoxyphenyl]-3α-methyl-7-methoxy-5-[(E)-1-propenyl]-2,3-dihydrobenzofuran

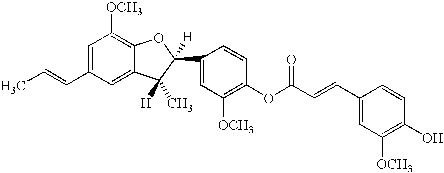

The above compound was prepared as per the general procedure by condensation of ferulic acid and dehydrodiisoeugenol.

¹H NMR (CDCl₃, 400 MHz) δₚₚₘ: −1.26 (d, 3H, J=13 Hz), 1.36 (d, 3H, J=6.7 Hz), 3.4 (m, 1H), 3.76 (s, 3H, 1×Ar—OCH₃), 3.84 (s, 3H, 1×Ar—OCH₃), 3.86 (s, 3H, 1×Ar—OCH₃), 5.11 (d, 1H, J=9.1 Hz), 6.0-6.2 (m, 1H, olefinic proton), 6.27 (d, 1H, J=2.0 Hz), 6.44 (d, 1H, J=15.9 Hz), 6.6-7.6 (m, Ar—H), 7.73 (d, 1H, J=15.9 Hz). TOF MS ES: −525 (M⁺+Na). Molecular formula: —C₃₀H₃₀O₇. M. R.: −115-118° C.

### Example 26

### 2-methoxy-4-(methoxycarbonyl)phenyl 3,4,5-trihydroxybenzoate

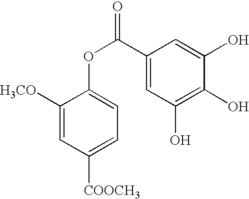

The above compound was prepared as per the general procedure by condensation of gallic acid and methyl vanillate.

¹H NMR (DMSO-d₆, 400 MHz) δₚₚₘ: −3.82 (s, 3H, 1×Ar—COOCH₃), 3.87 (s, 3H, 1×Ar—OCH₃), 7.0-7.8 (m, Ar—H), 9.4 (broad hump, 3H, 1×3-OH). TOF MS ES: −357 (M⁺+Na). Molecular formula: —C₁₆H₁₄O₈. M. R.: −193-196° C.

### General Procedure for the Preparation of Compounds of Benzofuran Lignan Derivatives

These compounds were prepared by the action of boron tribromide on Methyl (E)-3-[2-(4-hydroxy-3-methoxyphenyl)-7-methoxy-3-methoxycarbonyl-2,3-dihydro-1-benzofuran-5-yl]prop-2-enoate in dichloromethane at 0° C. for 2 hrs. The reaction mixture was decomposed by adding water. The organic layer washed with saturated solution sodium bicarbonate, water, brine and kept over anhydrous sodium sulphate. The organic layer concentrated to yield crude mass which was purified by radial chromatography with increasing concentration of ethyl acetate in petroleum ether.

### Example 27

### 5-[(E)-2-carboxyvinyl]-2β-(4-hydroxy-3-methoxyphenyl)-7-methoxy-2,3-dihydro-1-benzofuran-3α-carboxylic acid

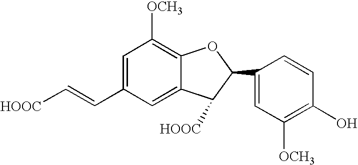

The above compound was prepared by the action of boron tribromide on Methyl (E)-3-[2-(4-hydroxy-3-methoxyphenyl)-7-methoxy-3-methoxycarbonyl-2,3-dihydro-1-benzofuran-5-yl]prop-2-enoate in dichloromethane at 0° C. for 2 hrs. The reaction mixture was decomposed by adding water. The organic layer washed with saturated solution sodium bicarbonate, water, brine and kept over anhydrous sodium sulphate. The organic layer concentrated to yield crude mass which was purified by radial chromatography with increasing concentration of ethyl acetate in petroleum ether. Molecular Formula: C₂₀H₁₈O₈. Viscous mass.

¹H NMR (CD₃OD, 500 MHz) δₚₚₘ: −3.66 (s, 3H, 1×Ar—OCH₃), 3.71 (s, 3H, 1×Ar—OCH₃), 4.2 (d, 1H, J=7 Hz), 5.86 (d, 1H, J=7 Hz), 6.21 (d, 1H, J=15.5 Hz), 6.5-7.2 (m, 5H, ArH), 7.47 (d, 1H, J=16.0 Hz). ¹³C NMR (CD₃OD, 125 MHz) δₚₚₘ: −50.37 (OCH₃), 51.54 (OCH₃), 55. 29, 86.85, 112.22 (ArH), 114.13, 114.69 (Olefinic carbon), 115.54 (ArH), 116.39 (ArH), 117.00 (ArH), 125.88, 128.10, 131.35, 141.39, 144.80 (Olefinic carbon), 144.99, 145.21, 149.03, 167.84 (>C═O), 171.15 (>C═O). DEPT: −50.37 (CH₃), 51.54 (CH₃), 55.29 (CH), 86.85 (CH), 112.22 (CH), 114.13 (CH), 114.69 (═CH), 115.54 (CH), 116.39 (CH), 117.00 (CH), 125.88 (>C<), 128.10 (>C<), 131.35 (>C<), 141.39 (>C<), 144.80 (═CH), 144.99 (>C<), 145.21 (>C<), 149.03 (>C<), 167.84 (>C═O), 171.15 (>C═O). TOF MS ES: −387 (M+H).

### Example 28

### 5-[(E)-2-carboxyvinyl]-7-hydroxy-2β-(4-hydroxy-3-methoxy phenyl)-2,3-dihydro-1-benzofuran-3α-carboxylic acid

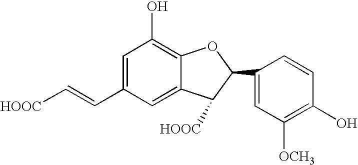

The above compound was prepared by the action of boron tribromide on Methyl (E)-3-[2-(4-hydroxy-3-methoxyphenyl)-7-methoxy-3-methoxycarbonyl-2,3-dihydro-1-benzofuran-5-yl] prop-2-enoate in dichloromethane at 0° C. for 2 hrs. The reaction mixture was decomposed by adding water. The organic layer washed with saturated solution sodium bicarbonate, water, brine and kept over anhydrous sodium sulphate. The organic layer concentrated to yield crude mass which was purified by radial chromatography with increasing concentration of ethyl acetate in petroleum ether.

¹H NMR (CD₃OD, 500 MHz) δₚₚₘ: −3.68 (s, 3H, 1×Ar—OCH₃), 4.16 (d, 1H, J=7 Hz), 5.84 (d, 1H, J=7 Hz), 6.19 (d, 1H, J=15.5 Hz), 6.5-7.0 (m, 5H, ArH), 7.45 (d, 1H, J=16.0 Hz). ¹³C NMR (CD₃OD, 125 MHz) δ ppm: −51.70 (OCH₃), 55. 21, 86.80, 112.40 (ArH), 114.83 (Olefinic carbon), 115.52 (ArH), 115.70 (ArH), 116.45 (ArH), 117.21 (ArH), 125.80, 128.36, 131.40, 141.26, 144.51 (Olefinic carbon), 144.89, 145.14, 148.80, 170.29 (>C═O), 171.28 (>C═O). DEPT: −51.70 (CH₃), 55.21 (CH), 86.80 (CH), 112.40 (CH), 114.83 (═CH), 115.52 (CH), 115.70 (CH), 116.45 (CH), 117.21 (CH), 125.80 (>C<), 128.36 (>C<), 131.40 (>C<), 141.26 (>C<), 144.51 (═CH), 144.89 (>C<), 145.14 (>C<), 148.80 (>C<), 170.29 (>C═O), 171.28 (>C═O). TOF MS ES: −373 (M+H). Molecular Formula: C₁₉H₁₆O₈. Viscous mass.

### Example 29

### (±)-2β-[4-O-(4-hydroxy cinnamoyl)-3-methoxyphenyl]-3α-methyl-7-methoxy-5-[(E)-1-propenyl]-2,3-dihydrobenzofuran

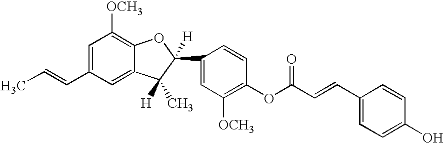

The above compound was prepared as per the general procedure by condensation of 4-dihydroxycinnamic acid with dehydrodiisoeugenol.

¹H NMR (CDCl₃, 400 MHz) δₚₚₘ: −1.41 (d, 3H, J=6.7 Hz), 1.87 (d, 3H, J=8.25 Hz), 3.48 (m, 1H), 3.82 (s, 3H, 1×Ar—OCH₃), 3.90 (s, 3H, 1×Ar—OCH₃), 4.24 (brs, 1H, —OH), 5.17 (d, 1H, J=9.1 Hz), 6.0-6.2 (m, 1H, olefinic proton), 6.36 (d, 1H, J=15.9 Hz), 6.50 (d, 1H, J=15.9 Hz), 6.6-7.6 (m, Ar—H),), 7.82 (d, 1H, J=15.9 Hz). TOF MS ES: −495 (M⁺+Na). Molecular Formula: C₂₉H₂₈O₆. M. R.: −145-148° C.

### BIOLOGICAL EVALUATION OF COMPOUNDS

Cell Lines: The cell lines used in this study were as follows: L929 (mouse fibroblast like cells), RAW 264.7 (mouse macrophage), U-937 (human histiocytic lymphoma), Jurkat (human T cell leukemia), MCF-7 (human breast cancer cell line), HeLa (human cervical cancer cell line); they were obtained from American Type culture collection (Manassas, Va., USA). L929, U-937, Jurkat, Raw 264.7 were cultured in RPMI 1640, while others in DMEM supplemented with 10% FBS, penicillin (1000 U/ml), and streptomycin (100 μg/ml).

Materials: All synthetic chemicals were obtained from commercial sources. Lipopolysaccharide (LPS), Bovine Serum Albumin (BSA), Phorbol Myristate Acetate (PMA), Propidium Iodide (PI), Actinomycin D (Act D), thiobarbituric acid, sulfanilamide, naphthylenediamine dihydrochloride, tetrazolium salt 3-(4,5-dimethylthiazol-2-yl)-2,5-diphenyltetrazolium bromide (MTT), DMSO etc were obtained from Sigma Aldrich Chemicals (St Louis, Mo., USA). Penicillin, streptomycin, neomycin, RPMI 1640 and DMEM medium, fetal bovine serum (FBS) were obtained from Gibco BRL. Purified recombinant human TNF-α (17.5 kDa) was purchased from R&D systems. The ED₅₀ value of TNF-α ranged from 0.02-0.05 ng/ml which corresponds to the specific activity of 2.5×10⁷-5×10⁷ Units/ml. COX-2 ELISA Kit was obtained from Zymed laboratories (Invitrogen immunodetection), Anti human p65 polyclonal antibody (Santa Cruz), Anti CD 54 FITC conjugate (BD), Anti PARP-FITC conjugate was from Novus Biologicals, The fluorescent reactive oxygen intermediate probe Dihydrorhodamine 123 (DHR 123) was purchased from Molecular Probes, the NF-κB Transcription Factor assay kit source was from BD Biosciences, Clontech. One step Access RT-PCR kit was purchased from Promega.

### Example 30

### Bioassay of Cytokine Production by RAW 264.7 Cells Using L929 Cell Line

Measurement of TNFα in culture medium or supernatant is performed using immunoassay and bioassay. Bioassay was used for the measurement of bioactive TNFα production in the culture medium.

TNFα secretion into the medium by LPS activated macrophage was assayed using L929 tumorigenic murine cells (ATCC) specifically sensitive to TNFα. L929 cell cytotoxicity assay was performed by a modified method (Sano et al., The Journal of Immunology, 1999, 163: 387-395 The Journal of Immunology, 1999, 163: 387-395) based on that described elsewhere (Flick D A, Gifford G E. J Immunol Methods. 1984 Mar. 30; 68(1-2): 167-175). Briefly, the L929 cells (log phase cells) were seeded into a flat bottom 96 well plates (6×10⁴/well) in 100 μl volume of RPMI 1640 containing 2% FCS and incubated overnight at 37° C. in a 5% CO₂ incubator. A working dilution of the culture supernatant collected from LPS (200 ng/ml) activated macrophage in a volume of 100 μl per well was first tested to obtain 70-75% cytotoxicity equivalent to 75 μg/ml of recombinant TNFα standard to the TNFα sensitive L929 cell line. After incubation of the L929 cells appropriate fixed dilution of the culture supernatant collected from the compound treated (10 μM) wells, containing the LPS stimulated macrophages in a volume of 100 μl with 2 μg/ml Actinomycin D (Act D) was taken and added to the L929 cells and the cells were incubated at room temperature for 15 min followed by 18 h incubation at 37° C. overnight with 5% CO₂. On the next day the medium was removed and the cells were stained with 0.2% (w/v) crystal violet for 10 minutes. The wells were gently rinsed with water, and 33% acetic acid (100 μl/well) was added to extract the retained crystal violet. The absorbance at 570 nm was finally measured.

### Example 31

### Nitrite Quantification

NO₂⁻ accumulation was used as an indicator of Nitric Oxide (NO) production in the medium as described previously (Green et al., 1982 Anal Biochem 126, 131-138). RAW 264.7 cells were plated at 5×10⁵ cells/ml, and stimulated with LPS (250 ng/ml) in the presence or absence of the test compounds for 24 h. The isolated supernatants were mixed with an equal volume of Griess reagent (1% sulfanilamide, 0.1% naphthylenediamine dihydrochloride, and 2% phosphoric acid) and incubated at room temperature for 15 min. NaNO₂ was used to generate a standard curve, and nitrite production was determined by measuring the optical density at 540 nm.

### Example 32

### Determination of Thiobarbituric Acid-Reactive Substances (TBARS)

Lipid peroxidation was assessed by the TBARS assay, which detects mainly malondialdehyde (MDA), a product of the peroxidation of polyunsaturated fatty acids and related esters. TBARS were measured by a modification of the method described previously, (Ohkawa et al., Anal. Biochem. 95:351-358. (1979)). Jurkat cells, 6×10⁶ cells in 2 ml were pretreated with either media or different concentrations of CAMVE (as described in the Figure legends) for 3 h and then stimulated with 1 nM TNF for 1 h. Cells were washed before undergoing three cycles of freeze-thawing in 200 μl of water. A 20 μl aliquot was subsequently removed for Bradford protein determination, and 800 μl of assay mix (0.4% (w/v) thiobarbituric acid, 0.5% (w/v) SDS, 9.4% (v/v) acetic acid, pH 3.5) was added to the remaining sample. Samples were incubated for 60 min at 95° C., cooled at room temperature, and centrifuged at 14,000×g for 10 min, and the absorbance of the supernatants was read at 532 nm against a standard curve prepared using the MDA standard (10 mM 1,1,3,3-tetramethoxypropane in 20 mM Tris-HCL, pH 7.4). Results were calculated as nmol of MDA equivalents/mg of protein and expressed as a percentage of matched control values. Untreated cells showed 0.568-0.08 nmol of TBA-reactive substances/mg protein (subtracting the background absorbance obtained by heating 800 μl of assay mix plus 200 μl water).

### Example 33

### Measurement of Reactive Oxygen Intermediate ROI

The production of ROI in cells treated with TNFα or LPS was determined by flow cytometer as described by (Manna, S. K. et. al., J. Immunol. (1999) 162(3):1510-1518). Briefly Jurkat cells (5×10⁵ cells in one ml) were incubated either with RPMI 1640 medium supplemented with 10% FBS or with complete media containing different concentrations of CAMVE for 1 h at 37° C. Cells were then stimulated with 1 nM of TNF for 4 h. After incubation, the cells were washed with D-PBS, and resuspended in 1 ml D-PBS. To detect ROI production, cells were exposed to Dihydrorhodamine 123 (5 mM stock) at a final concentration of 1 μM for 1 hr at 37° C. with moderate shaking (100 rpm) and then washed with D-PBS three times and resuspended in 1 ml of D-PBS. Rhodamine 123 fluorescence intensity resulting from Dihydrorhodamine 123 oxidation was measured by FACS Calibur (Becton Dickinson) with excitation at 488 nm and was detected between 515 and 550 mm. Data was analyzed using Cell Quest software (Becton Dickinson).

### Example 34

### Preparation of Nuclear Extracts

Cell pellet was resuspended in lysis buffer (10 mM HEPES, pH 7.9, 1.5 mM MgCl₂, 10 mM KCl, 1 mM PMSF, 1 mMDTT, 0.5% NP 40, 0.1 mM EGTA and 0.1 mM EDTA) and allowed to swell on ice for 15 min, followed by centrifugation at 3300×g for 20 min. The cell pellet was resuspended in a volume of lysis buffer equal to the cell pellet volume. The cell suspension was slowly drawn down into a syringe and ejected the content in a single stroke. Disrupted cells were incubated for 15 min on ice, and the disrupted cell suspension was centrifuged at 10,000×g for 20 min at 4° C. Nuclear pellet was resuspended in a volume of extraction buffer (20 mM HEPES; pH 7.9, 25% glycerol, 1.5 mM MgCl₂, 420 mM NaCl, 0.1 mM EDTA, 0.1 mM EGTA, 1 mM PMSF and 1 mM DTT) and incubated on ice for 30 min. The nuclear suspension was centrifuged at 21,000×g for 15 min at 4° C. and the supernatant was collected as nuclear extract and stored at −70° C. Protein concentration was estimated using standard Bradford method.

### Example 35

### NF-κB Activation Assay

To determine NF-κB activation, transcription profiling was done with the BD Mercury Transfactor kit obtained from BD Biosciences. This method provides rapid, high-throughput detection of specific transcription factors eg NFκB in the nuclear extract. Using an enzyme-linked immunosorbent assay (ELISA)-based format, the Transcription Factor kit detects the DNA binding by specific transcription factors. This method is faster, easier, and significantly more sensitive than eletrophoretic mobility shift assays [SMSA].

The assay was performed by using wells coated with oligonucleotides having the consensus DNA binding sites for the specific transcription factors. 50 μg of the nuclear extract proteins were incubated in the wells precoated with their specific oligonucletides, and allowed the activated NFκB to bind to their consensus sequence. Bound transcription factor was detected by a specific Primary Antibody. A horseradish peroxidase conjugated Secondary Antibody was then used to detect the bound Primary Antibody. After addition of the substrate, the Absorbance was recorded at 655 nm.

### Example 36

### Nuclear Translocation of p65 NF-kB by Immunocytochemistry

Hela cells grown on cover slips were washed with 0.1M potassium phosphate buffer (pH 7.4) and fixed with 4% formaldehyde in 0.1M potassium phosphate buffer (pH 7.4) for 1 h at room temperature. The cells were permeabilized with 0.1% Triton X-100 in PBS for 1 h. It was then incubated with rabbit anti human p65 polyclonal antibody (Santa Cruz) at room temperature for 1 h, and then stained with secondary FITC conjugated goat anti rabbit IgG antibody (Sigma) for 1 h at room temperature. After counterstaining for nuclei with DAPI or Hoechst for 5 mins slides were analyzed under a fluorescence microscope (Labophot-2. Nikon, Tokyo, Japan).

### Example 37

### Nuclear Translocation of p65 by Flow Cytometry

The assay was performed as described previously (Blaecke, A., Yves, D., Herbault, N., Jeannin, P., Bonnefoy, J. V., Beck, A., Aubry, J. P. (2002) Cytometry 48, 71-79). Briefly after stimulation, cells were washed twice with PBS. Nucleus was prepared by incubating the cells with 200 ul Pipes-Triton buffer for 30 min at 4° C. Nuclei staining was performed using mouse anti-human NFκB p65 mAb (Santa Cruz) or with the matching isotype control at 3 μg/ml for 30 mins. After washing, the nuclei were incubated with secondary FITC conjugated goat anti-mouse antibody (Sigma) for additional 30 mins at 4° C. and analysed using FACS.

### Example 38

### COX-2 Protein and Gene Expression

Quantitative detection of Cox 2 protein expression by activated cells was done by an enzyme-linked immunosorbent sandwich assay using Zymed COX-2 ELISA kit. 1×10⁷ cells were treated with different concentrations of CAMVE and stimulator and incubated at 37° C. for 12 hrs. After stimulation, cells were rinsed twice with ice cold PBS, and 100 μl of lysis buffer (150 mM NaCl, 50 mM Tris-HCl pH7.5,500 μM EDTA, 100 uM EGTA, 1.0% Triton X-100 and 1% sodium deoxycholate, 1 mM PMSF, 10 μg/ml leupeptin, 10 μg/ml aprotinin) was added to the pellet. Lysates were sonicated for 20s on ice and centrifuged at 10,000×g for 10 min to sediment the particulate material. The protein concentrations of the supernatants were measured by Bradford assay (Anal. Biochem. 72:248-254 (1976). 200 μg/100 μl of the protein was assayed per sample according to the kit protocol. Briefly, 100 μl of the sample and the standard were put in the pre antibody coated wells and incubated at 1 h at 37° C. After three washes 100 μl of the HRP conjugated antibody was added and incubated for 30 m is at 4° C. After washing, 1000 of the TMB substrate was added and incubated for 30 min at room temperature in dark. The reaction was stopped and the absorbance was read at 450 nm.

### Example 39

### ICAM 1 (CD 54) Gene Expression

Cells were pretreated with different concentrations of CAMVE for 3 h and then treated with 0.1 nM of TNF or 100 ng/ml SA-LPS for 12 h at 37° C. in a CO₂ incubator. Extent of ICAM 1 expression was detected by staining the washed cells with FITC-labeled monoclonal antibody which binds to the cells expressing the CD 54 (ICAM 1). Unbound FITC-conjugated antibody is then washed from the cells and the cells were resuspended in 0.5 ml of 1% paraformaldehyde, and analyzed using Flow Cytometer (B D FACS Calibur). Cells CD54 structure is fluorescently stained, with the intensity of staining is directly proportional to the density of CD54.

### Example 40

### PARP Cleavage Assay Using Flow Cytometry

Extent of PARP cleavage is determined using polyclonal antibody specifically recognizing the 85 kDa fragment of cleaved PARP (NSB 699 Novus Biologicals) and can be used as a marker for detecting apoptotic cells. Treated cells were fixed with 70% chilled ethanol and permeabilized for 30 min at RT (PBS+0.5% BSA+0.02% NaN₃+0.5% saponin) and stained with anti PARP-FITC (10 μl/10⁶ cells) for one hour at RT. Cells were washed twice with wash buffer (PBS+1% heat inactivated FBS) and analysed using FACS.

### Example 41

### Cytotoxicity Assay

Cytotoxicity was assayed by the modified tetrazolium salt 3-(4,5-dimethylthiazol-2-yl)-2,5-diphenyltetrazolium bromide (MTT) assay as described by Plumb, J A et al. (Cancer Res. 1989 Aug. 15; 49(16):4435-4440).

### Example 42

### Cell Cycle Analysis

Cells (log phase culture) were treated with vehicles alone (similar volumes of DMSO) or with the compound (various concentrations) to be tested for 24 h. Untreated cells were also included in this experiment for comparison. After treatments, the cells were harvested and washed with cold EDTA/PBS (5 mmol/L). Cells were then resuspended in cold EDTA/PBS (300 μl) and 100% chilled ethanol (700 μl), vortexed, and incubated at room temperature for 1 h. Samples were centrifuged at 200×g for 5 minutes and the supernatant was removed. A solution containing propidium iodide (100 μg/ml) and RNase A (1 mg/ml) was added to the samples and incubated for 1 h at room temperature. Samples were then transferred to 12×75 mm polystyrene tubes and analyzed on flow cytometer. Flow cytometry analyses were done on FACSCalibur (Becton Dickinson, San Jose, Calif.) and data were analyzed using CellQuest analysis software.

### Example 43

### RNA Extraction

Total RNA was isolated using the standard TRIzol method (Gibco BRL). Briefly, 3×10⁶ cells were treated with different concentrations of the compounds as indicated in the legends to Figures and after harvesting the cell pellet was resuspended in 1 ml of TRIzol with repeated pipetting. The homogenized sample was incubated at RT for 5 min to permit complete dissociation of the nucleoprotein complexes. 200 μl of chloroform was added and the tubes were shaken vigorously for 15 sec by hand and then incubated at RT for 3 mins. The samples were then centrifuged at 10,000 RPM for 15 mins at 4° C. The aqueous phase was transferred to a fresh tube and 500 μl of Isopropyl alcohol was added, followed by incubation at RT for 10 mins. The samples were centrifuged at 10,000 RPM for 10 min at 4° C. and the RNA pellet was washed with 1 ml of 75% ethanol followed by centrifugation at maximum speed at 4° C. for 10 mins. Washing was repeated once more and after removing the supernatant, the RNA pellet was dried and dissolved in 20 μl of RNase free water (Promega). To ensure total resuspension, tubes were incubated at 55-60° C. for 10 mins. The samples were aliquoted and stored at −70° C.

### Example 44

### Semi-Quantitative Reverse Transcriptase (RT)-PCR

Changes in gene expression were verified by semi-quantitative RT-PCR using GAPDH as an internal normalization standard. 1 μg of total RNA (quantified by spectrophotometer) was used to reverse transcribe into cDNA. One step Access RT-PCR kit (Promega) was used for the synthesis of c-DNA followed by the amplification of the gene of interest using gene specific primers. Briefly, 50 μl reaction mixture including 10 μl of 5×AMV/Tfl reaction buffer, 0.2 mM dNTP mix, 50 pmol each of forward and reverse primers, 1 mM of MgSO₄ and 1 μg of RNA sample, was subjected to 28 PCR cycles (First strand cDNA synthesis: reverse transcription at 48° C. for 45 minutes, AMV RT inactivation and RNA/cDNA/primer denaturation at 94° C. for 2 minutes. Second strand synthesis and PCR amplification: denaturation at 94° C. for 30 sec, annealing at primer specific temperature for 1 minute and polymerization at 68° C. for 2 minutes. Amplification products were separated by agarose gel electrophoresis (2%) and visualized by ethidium bromide staining. The primer sequences and product sizes were as follows:

| 1) Cyclin D1: 402 bp | |
|---|---|
| 5′-ACCTGGATGCTGGAGGTCTG-3′ | {forward}; |
| 5′-GAACTTC-ACATCTGTGGCACA-3′ | {reverse} |
| (Kwon,Y.K. et al. (2004) Internationai J of | |
| Oncology 26: 1597-1603, 2005) | |
| 2) GAPDH: 239 bp | |
| 5′-TGATGACATCAAGAAGGTGGTGAA-3′ | {forward}; |
| 5′-TCCT-TGGAGGCCATGTGGGCCAT-3′ | {reverse}. |

### Results and Discussions

All compounds used in these studies were dissolved in DMSO as 10 mM stock solution and further dilutions was made in complete medium. The concentration of compounds used and the duration of exposure had minimal effect on the viability of these cells as determined by trypan blue dye exclusion test.

### Example 45

### Cytotoxity of L929 Cells Incubated with Supernatant from LPS or LPS+Synthetic Compound Stimulated Macrophage Culture

Tumor necrosis factor-α can cause direct cytotoxicity to the lung fibroblast cell line (L929). Bioassay of TNF α is designated on the basis of this cytotoxicity of TNF α, which can be used for the identification of murine TNF α activity in tissue culture supernatants. Culture supernatant collected from the LPS activated macrophage produced almost 76% cytotoxicity as shown in Table 1. Culture supernatant collected from the synthetic molecules treated well containing LPS stimulated macrophage modulated the extent of cytotoxicity to the L929 cells (see Table 1).

Most of the compounds were able to reduce the cytotoxicity to some extent. However, cytotoxicity of the L929 cells by TNF α was highly reduced by few of the synthetic molecules which suggest that their treatment could inhibit the production of TNF α from macrophages.

TABLE 1
Cytotoxity of L929 cells incubated with supernatant from LPS or LPS + Synthetic
compound stimulated macrophage culture.

| | PERCENTAGE CELL |
|---|---|
| TREATMENT | SURVIVAL ± S.D. |
| L929 + ActD control | 100 |
| L929 + ActD + LPS + DMSO | 99.078 ± 0.490 |
| L929 + ActD + Sup from LPS treated macrophage | 24.682 ± 1.242 |
| L929 + Act D + TNF (75 pg/ml) control | 25.700 ± 0.812 |
| L929 + ActD + Sup from(LPS + compound 1) treated macrophage | 73.155 ± 1.773 |
| L929 + ActD + Sup from(LPS + compound 2) treated macrophage | 68.003 + 1.531 |
| L929 + ActD + Sup from(LPS + compound 3) treated macrophage | 67.048 ± 2.301 |
| L929 + ActD + Sup from(LPS + compound 4) treated macrophage | 69.084 ± 1.491 |
| L929 + ActD + Sup from(LPS + compound 5) treated macrophage | 67.812 ± 0.866 |
| L929 + ActD + Sup from(LPS + compound 6) treated macrophage | 66.571 ± 1.485 |
| L929 + ActD + Sup from(LPS + compound 7) treated macrophage | 65.299 ± 1.114 |
| L929 + ActD + Sup from(LPS + compound 8) treated macrophage | 67.844 ± 1.251 |
| L929 + ActD + Sup from(LPS + compound 9) treated macrophage | 51.081 ± 2.768 |
| L929 + ActD + Sup from(LPS + compound 10) treated macrophage | 51.559 ± 1.397 |
| L929 + ActD + Sup from(LPS + compound 11) treated macrophage | 52.417 ± 1.510 |
| L929 + ActD + Sup from(LPS + compound 12) treated macrophage | 51.304 ± 1.208 |
| L929 + ActD + Sup from(LPS + compound 13) treated macrophage | 50.636 ± 1.603 |
| L929 + ActD + Sup from(LPS + compound 14) treated macrophage | 50.954 ± 1.908 |
| L929 + ActD + Sup from(LPS + compound 15) treated macrophage | 50.859 ± 1.843 |
| L929 + ActD + Sup from(LPS + compound 16) treated macrophage | 49.046 ± 1.992 |
| L929 + ActD + Sup from(LPS + compound 17) treated macrophage | 51.877 ± 1.429 |
| L929 + ActD + Sup from(LPS + compound18) treated macrophage | 50.350 ± 1.575 |
| L929 + ActD + Sup from(LPS + compound 19) treated macrophage | 51.590 ± 1.575 |
| L929 + ActD + Sup from(LPS + compound 20) treated macrophage | 51.908 + 1.100 |
| L929 + ActD + Sup from(LPS + compound 21) treated macrophage | 52.290 ± 1.251 |
| L929 + ActD + Sup from(LPS + compound 22) treated macrophage | 65.553 ± 1.326 |
| L929 + ActD + Sup from(LPS + compound 23) treated macrophage | 51.209 ± 1.554 |
| L929 + ActD + Sup from(LPS + compound 24) treated macrophage | 50.286 ± 1.284 |
| L929 + ActD + Sup from(LPS + compound 25) treated macrophage | 53.690 ± 2.047 |
| L929 + ActD + Sup from(LPS + compound 26) treated macrophage | 58.810 ± 2.147 |
| L929 + ActD + Sup from(LPS + compound 27) treated macrophage | 46.533 ± 1.020 |
| L929 + ActD + Sup from(LPS + compound 28)treated macrophage | 66.444 ± 1.106 |
| L929 + ActD + Sup from(LPS + compound 29)treated macrophage | 50.604 ± 1.492 |

TNF sensitive L929 cells were treated with supernatant collected from either LPS or LPS + Synthetic compound treated RAW 264.7 cells as described. Cell viability was assessed by crystal violet staining. Data is represented as percentage cell survival and each value shows the mean ± S.D. of triplicate samples.

### Example 46

### Effect of the Synthetic Compounds on the Nitrite Production by LPS Stimulated Macrophages

To estimate the anti-inflammatory effects of all the synthetic molecules listed in this study, we measured the accumulation of nitrite, the stable metabolite of NO, in the culture media using Griess reagent. As listed in the Table 2. LPS drastically increased the levels of NO in the culture medium when compared to the basal levels, and this induction was significantly controlled when LPS treatment was given in the presence 10 μM of the synthetic molecules. The concentration of LPS induced nitrite accumulation, with or without the presence of any molecule is listed in Table 2. From the data shown, few of the molecules showed very promising results and in order to carry out further detailed studied we selected one of the best amongst them (CAMVE).

TABLE 2
Effect of Synthetic compounds on the nitrite production by
LPS stimulated macrophages.

| | CONCENTRATION |
|---|---|
| | OF NITRITE |
| TREATMENT | μM ± SD |
| Macrophage + DMSO | 0.782 ± 0.102 |
| MACROPHAGE + LPS + DMSO | 14.560 ± 0.133 |
| MACROPHAGE + LPS + compound1 | 1.849 ± 0.214 |
| MACROPHAGE + LPS + compound 2 | 2.404 ± 0.168 |
| MACROPHAGE + LPS + compound 3 | 3.022 ± 0.300 |
| MACROPHAGE + LPS + compound 4 | 3.093 ± 0.437 |
| MACROPHAGE + LPS + compound 5 | 3.449 ± 0.315 |
| MACROPHAGE + LPS + compound 6 | 3.204 ± 0.168 |
| MACROPHAGE + LPS + compound 7 | 3.760 ± 0.546 |
| MACROPHAGE + LPS + compound 8 | 3.893 ± 0.133 |
| MACROPHAGE + LPS + compound 9 | 7.582 ± 0.868 |
| MACROPHAGE + LPS + compound 10 | 6.849 ± 0.806 |
| MACROPHAGE + LPS + compound 11 | 5.604 ± 0.668 |
| MACROPHAGE + LPS + compound 12 | 9.582 ± 0.482 |
| MACROPHAGE + LPS + compound 13 | 8.004 ± 0.379 |
| MACROPHAGE + LPS + compound 14 | 8.382 ± 0.278 |
| MACROPHAGE + LPS + compound 15 | 8.271 ± 0.342 |
| MACROPHAGE + LPS + compound 16 | 8.449 ± 0.234 |
| MACROPHAGE + LPS + compound 17 | 8.404 ± 0.315 |
| MACROPHAGE + LPS + compound 18 | 7.960 ± 0.437 |
| MACROPHAGE + LPS + compound 19 | 8.227 ± 0.115 |
| MACROPHAGE + LPS + compound 20 | 8.493 ± 0.267 |
| MACROPHAGE + LPS + compound 21 | 8.293 ± 0.371 |
| MACROPHAGE + LPS + compound 22 | 3.982 ± 0.301 |
| MACROPHAGE + LPS + compound 23 | 8.338 ± 0.204 |
| MACROPHAGE + LPS + compound 24 | 8.582 ± 0.214 |
| MACROPHAGE + LPS + compound 25 | 7.871 ± 0.168 |
| MACROPHAGE + LPS + compound 26 | 6.138 ± 0.567 |
| MACROPHAGE + LPS + compound 27 | 5.027 ± 0.200 |
| MACROPHAGE + LPS + compound 28 | 7.604 ± 0.204 |
| MACROPHAGE + LPS + compound 29 | 8.382 ± 0.454 |

RAW 264.7 cells were plated at 5 × 10⁵ cells/ml, and stimulated with LPS (200 ng/ml) in the presence or absence of test compounds for 24 h. The culture supernatants were subsequently isolated and analyzed for nitrite production as described in the “Materials and Methods”. Each value shows the mean ± S.D. of triplicate determinations.

### Example 47

### Effect of CAMVE (Compound 1) on LPS Induced Nitrite Production

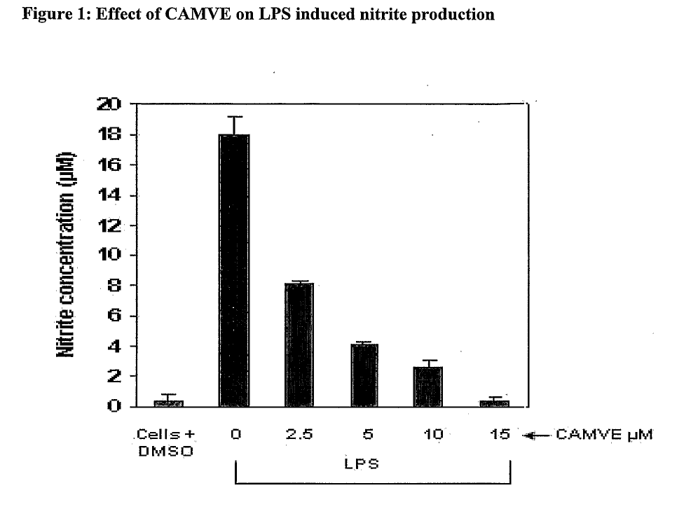

To investigate the effect of CAMVE on NO production, we measured the accumulation of nitrite, the stable metabolite of NO, in the culture media using Griess reagent. To investigate the effect of CAMVE on NO production, Raw 264.7 cells, pretreated with indicated concentrations of CAMVE for 1 h were incubated with LPS (250 ng/ml) for 24 h. As shown in FIG. 1, LPS alone evoked nitrite production significantly when compared to the naive control, and this induction was inhibited by CAMVE treatment in a dose-dependent manner.

### Example 48

### CAMVE Blocks TNF Induced ROI Generation and Lipid Peroxidation

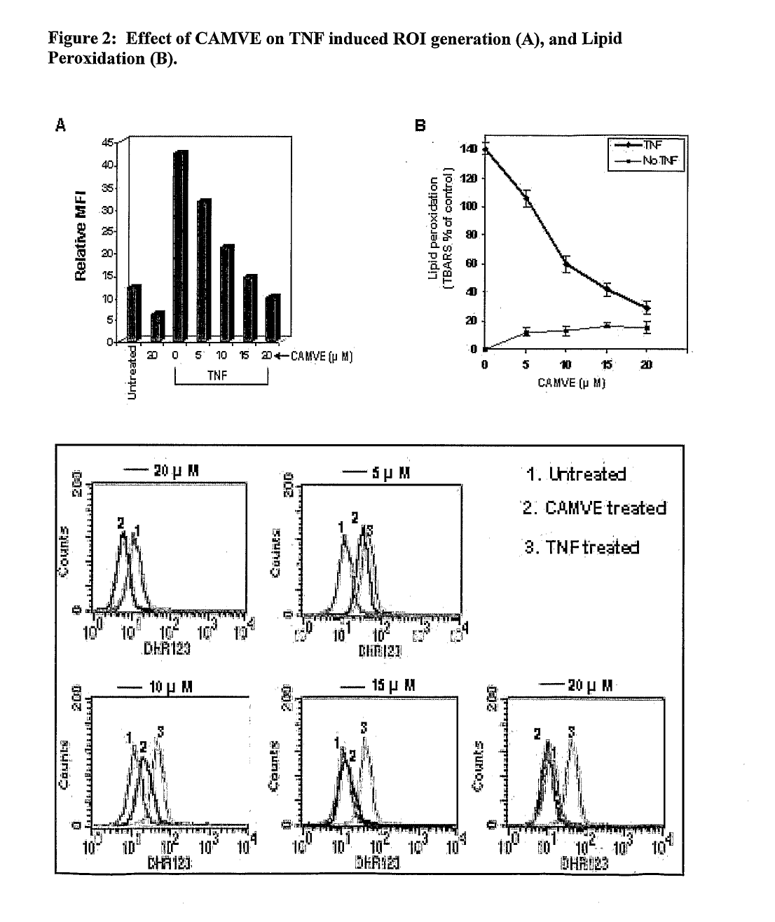

Previous reports have shown that TNF activates NF-43 through generation of ROI (Manna, S. K. et. al., J Immunol. (1999) 162(3):1510-1518; Li, N., and Karin, M. FASEB J. (1999) 13(10):1137-1143]. It's also been reported that ester derivatives of caffeic acid are known as structural relative of flavanoids and displays antioxidant activity (Kimura, Y., et. al; 1985 Chem Pharm Bull (Tokyo) 33(5):2028-2034). Whether CAMVE could suppress NF-κB activation through suppression of ROI generation was examined by flow cytometry. As shown in FIG. 2A, TNF induced ROI generation was suppressed on pretreatment of cells with CAMVE in a dose dependent fashion. Because lipid peroxidation has also been implicated in TNF-induced NF-κB activation (Bowie, A. G., P. N. Moynagh, and L. A. J. O'Neill. (1997) J. Biol. Chem. 272, 25941), we also examined the effect of CAMVE on TNF induced lipid peroxidation. Results in FIG. 2B show that TNF induced lipid peroxidation in Jurkat cells, and this was significantly suppressed by CAMVE in a dose dependent manner. Thus, it is quite likely that CAMVE could prevent oxidative stress induced by various agents and also block TNF signaling through suppression of ROI generation and lipid peroxidation.

### Example 49

### CAMVE Inhibits TNF or LPS Induced NF-κB Activation

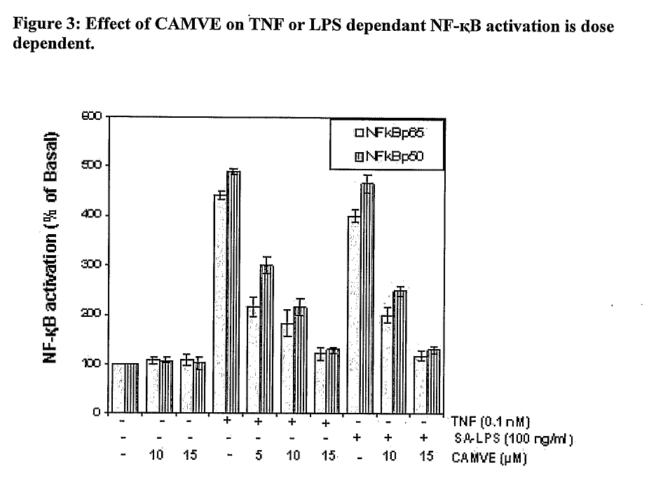

Jurkat cells were pretreated with the indicated concentrations of CAMVE for 3 h and then stimulated with 0.1 nM TNF or 100 ng/ml SA-LPS for 30 min; nuclear extracts were prepared and assayed for NF-κB by using ELISA based method. As shown in FIG. 3, TNF activated NF-κB almost 4.35 fold (when probed with antibody against p65) and 4.90 fold (when probed with antibody against p50), and CAMVE inhibited this activation in a concentration dependent manner, with maximum inhibition achieved at 15 μM. Similarly, LPS induced NF-κB activation was also blocked by CAMVE as shown in the FIG. 3. Without being bound by this theory, this result suggests that CAMVE may act at a step where TNF and LPS converge in the signal transduction pathway.

Various combinations of Rel/NF-κB proteins can constitute an active NF-κB heterodimer that binds to specific sequences in DNA. In this assay, the use of specific antibodies against p65 and p50 subunits of the NF-κB heterodimer bound to it's specific oligo coated wells, suggests that the TNF-activated complex consisted of p50 and p65 subunits of the NF-κB transcription factor. Furthermore, the use of competitor oligos having the same DNA sequence as the oligo-coated wells, decreases the signal because NF-κB binding decreases as it competes away from the oligo-coated surface of the TransFactor well, indicating the specificity for NF-κB.

### Example 50

### Inhibition of NF-κB Activation by CAMVE is not Cell Type Specific

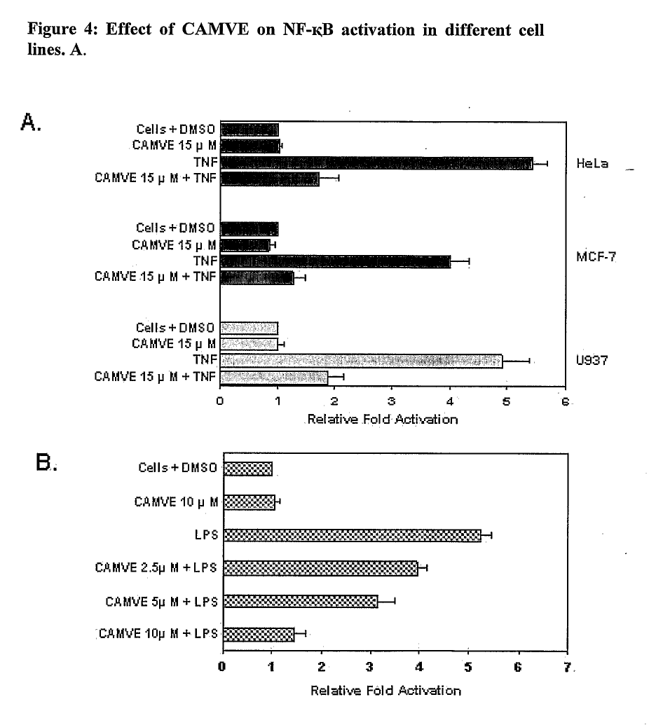

As NF-κB activation pathways differ in different cell types, we therefore studied whether CAMVE affects other cell types as well. It has been demonstrated that distinct signal transduction pathways could mediate NF-κB induction in epithelial and lymphoid cells (Bonizzi, G., Piette, J., Merville, M. P., and Bours, V. (1997). J. Immunol. 159:5264-5272). All the effects of CAMVE was mainly carried out in Jurkat, a human T cell leukemia. In another set of experiments, we found that CAMVE blocks TNF-induced NF-κB activation in HeLa (human cervical cancer), MCF-7 (human breast cancer), U937 (human histiocytic lymphoma) cells as shown in FIG. 4A. these results suggest that the effect of CAMVE is not restricted to leukemic T cells but also suppresses NF-κB activation in other cell types.

As all the cell lines tested are of human origin, we also examined the effect of CAMVE on LPS induced NF-κB activation in murine RAW 264.7 cells. Results shown in FIG. 4B, indicates that CAMVE inhibited NF-κB activation in murine cells too and it's potency was not significantly different from that of human cells. Furthermore, it is well known that NF-κB is an important target for the inducibility of iNOS gene expression by LPS, the inhibition of LPS (100 ng/ml) induced NF-κB activation by CAMVE is very much consistent with the NO (FIG. 1) data. Thus the inhibition of LPS induced NF-κB activation positively correlates to the degree of inhibition of Nitric oxide (NO) production induced by LPS in RAW 264.7 cells.

### Example 51

### CAMVE Inhibits TNF or LPS Induced Nuclear Translocation of p65

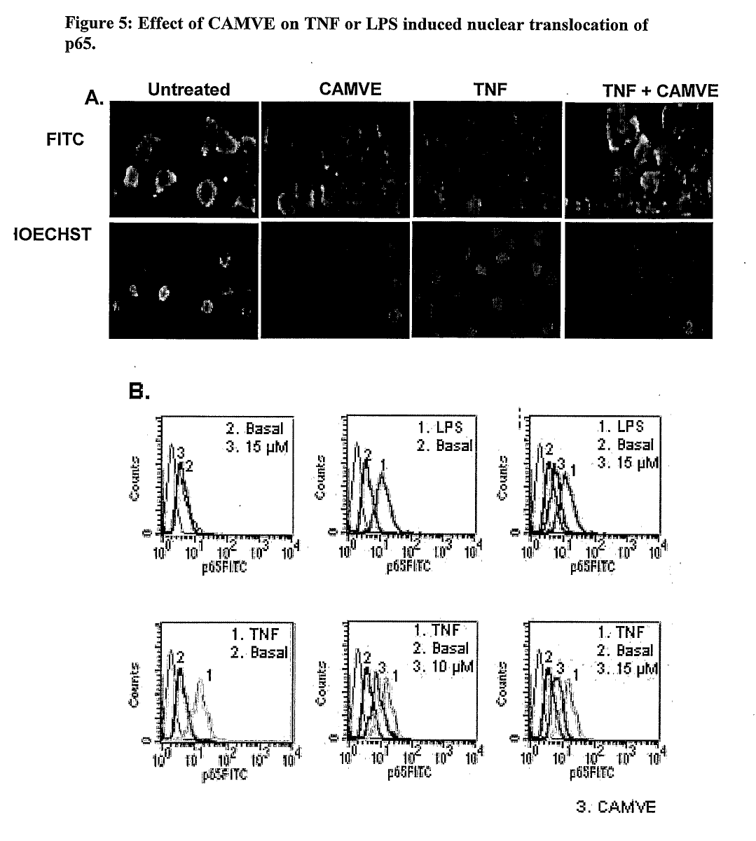

Analysis of the p65 translocation was done using flow cytometry and immunofluorescence. Inhibition of TNF induced p65 nuclear translocation by CAMVE in HeLa cell line was also proved by immunofluorescence wherein CAMVE pretreated cells did not show p65 signal, otherwise shown by TNF alone treated cells, in the nucleus FIG. 5A.

Flow cytometry analysis of NF-κB translocation in nuclei purified from treated cells is illustrated in FIG. 5 B. Nuclei extracted from Jurkat cells pretreated with CAMVE and stimulated with (0.1 nM) TNF or (100 ng/ml) SA-LPS were stained for p65. Staining of p65 in the nuclei of unstimulated cells differed only slightly from the isotype control, indicating a low basal activity of the cells. Basal values were not altered by incubation with CAMVE alone. In our assay, TNF or LPS significantly increased p65 translocation compared to the untreated control (7 fold in case of TNF and 6 fold in case of LPS). The TNF or LPS mediated p65 translocation was blocked with CAMVE pretreatments as shown in FIG. 5B. Nuclei population was gated on the basis of PI staining (1 μg/ml), after doublet elimination by FL-2 Area vs FL-2 Width measurements.

### Example 52

### CAMVE inhibits NF-κB Regulated Expression of Genes Associated with Inflammation and Carcinogenesis

Because CAMVE has shown to inhibit TNF and LPS induced NF-κB activation, we examined the expression of NF-κB regulated genes for example adhesion molecule ICAM 1 and COX 2 both known to be major players during inflammation.

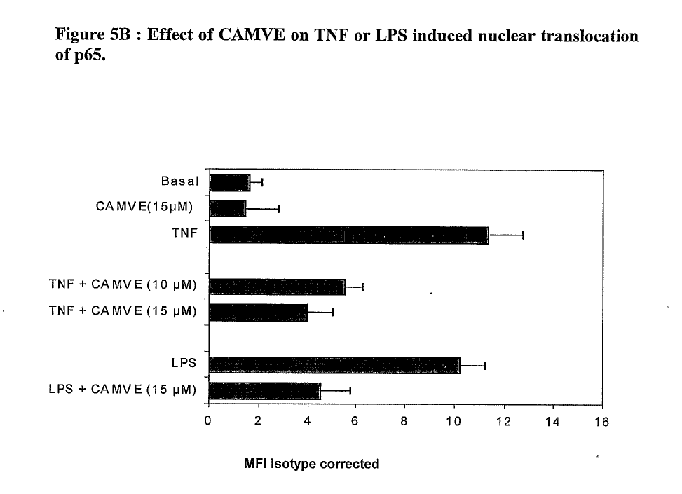

Cells treated with different concentrations of CAMVE for 3 h and then stimulated with either TNF (0.1 nM) or SA-LPS (100 ng/ml)) for 12 h. After harvesting the treated cells, equal amounts of the cellular lysates were checked for COX-2 protein expression by using COX-2 ELISA kit (Zymed). As shown in FIG. 6, COX2 expression induced by NF-κB activating agents was decreased with increasing concentration of CAMVE treatment. CAMVE alone did not show any induction of COX 2 protein.

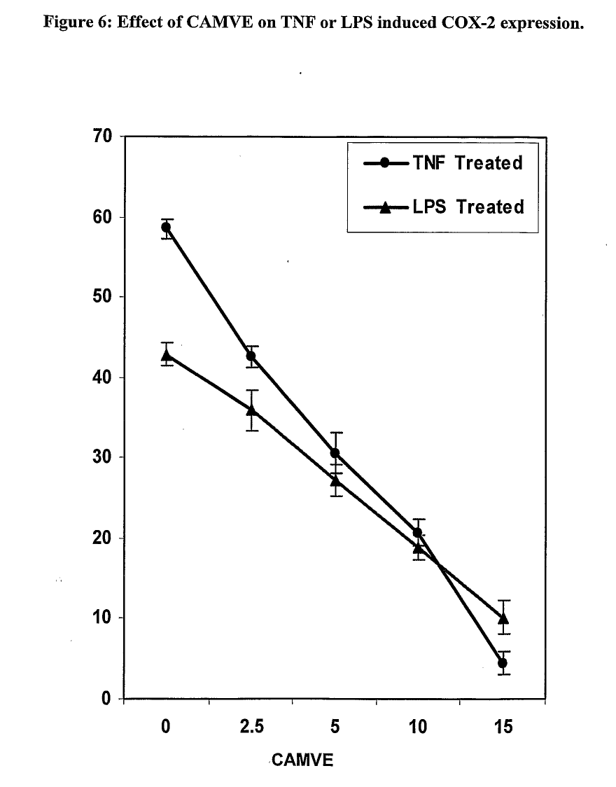

Adhesion molecule ICAM 1 expression on TNF or LPS stimulated cells was analyzed by FACS. Cells were pretreated with CAMVE for 3 h and then incubated with TNF (0.1 nM) or SA-LPS (100 ng/ml) for 12 h. As shown in FIG. 7, TNF or LPS stimulated cells showed a clear-cut increased in ICAM1 expression compared to the untreated control. This induced expression was blocked in CAMVE pretreated cells as shown in the figure. Basal ICAM1 expression was not altered by incubation with CAMVE alone.

### Example 53

### CAMVE Potentiates Apoptotic Effects of TNF and Chemotherapeutic Agents

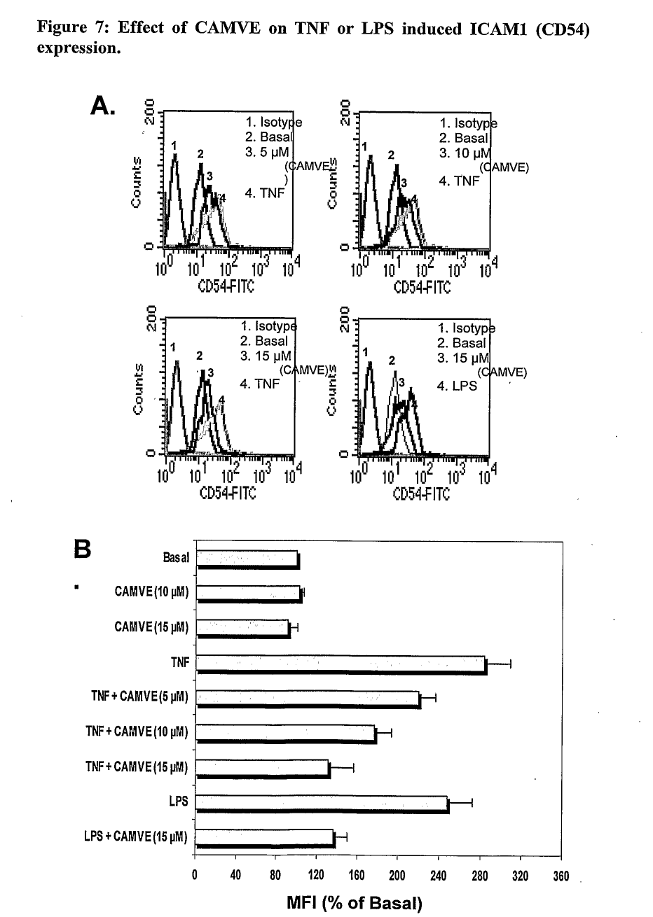

Out of the almost 17 members of the TNF superfamily, TNF is probably the most potent inducer of apoptosis. TNF activates both cell-survival and cell-death mechanisms simultaneously. Activation of NF-kB-dependent genes regulates the survival and proliferative effects pf TNF, whereas activation of caspases regulates the apoptotic effects. (Rath, P. C., and Aggarwal, B. B. (1999) J. Clin. Immunol 19:350-364). Because NF-κB regulated gene products, known to have antiapoptotic properties, can also suppress TNF and chemotherapy induced apoptosis, we examined the effects of CAMVE on the apoptotic effects of TNF and other chemotherapeutic drugs. Jurkat cells were treated with variable concentrations of TNF for 72 h either in the absence or presence of 10 μM of CAMVE and then examined for cytotoxicity by the MTT method. Results in FIG. 8A1, show that the cytotoxic effects of TNF in Jurkat cells were dose dependent and it was further potentiated by treatment of cells with 10 μM of CAMVE. To show that the cell death mediated by CAMVE was apoptosis and not necrosis, caspase activation in the form of PARP cleavage was examined using FACS. As shown in FIG. 8A2, TNF induced 16.28% of the cells to undergo apoptosis, and CAMVE pretreated cells showed significant potentiation of PARP cleavage in a dose dependent manner. Furthermore, in order to know the effect of chemotherapeutic drugs on NF-κB activated cells, SA-LPS (100 ng/ml) activated Jurkat cells were pretreated with 10 μM CAMVE for 3 h, were incubated with 1 μM each of cis-platin, doxorubicin, taxol or vincristine for 72 h and cell viability was assessed by the MTT method. As shown in FIG. 8B., cytotoxicity induced by various chemotherapeutic agents in NF-κB expressing cells was significantly enhanced by CAMVE pretreatment.

### Example 54

### CAMVE Induced Differential Cytotoxicity in Different Tumor Cell Lines

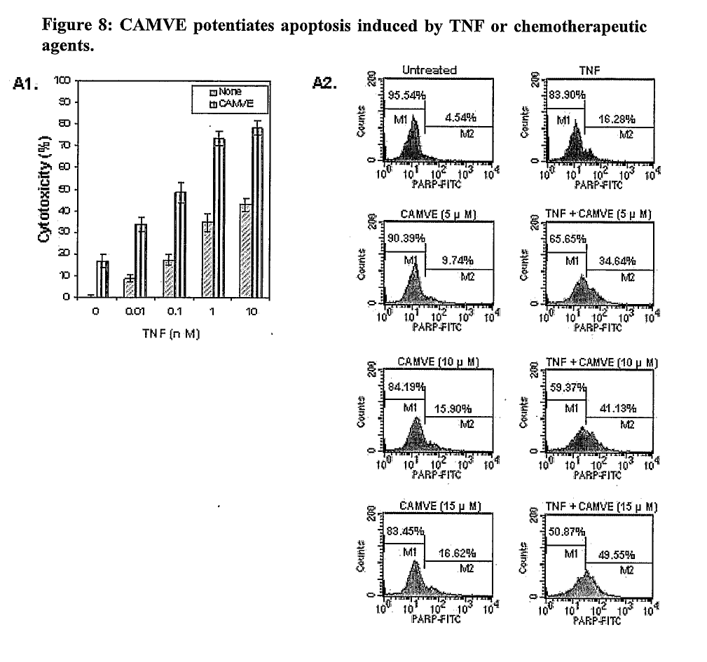

As polyphenolic compounds at higher concentrations are also known to alter the redox state and induce apoptosis in transformed cells (Chiao, C., Carothers, A. M., Grunberger, D., Solomon, G., Preston, G. A. and Barrett, J. C. (1995) Cancer Res 55, 3576-3583), we investigated as to whether CAMVE at higher concentration (30 μM) is also able to mediate cell death in tumor cells derived from different tissue background. The viability of cells after 72 h with or without CAMVE treatment was analyzed using the MTT assay. As shown in FIG. 9 it is clear that CAMVE mediated cell death is very much cell type or lineage dependent. U937 (human histiocytic lymphoma) cell line seem to be most sensitive to CAMVE treatment. Thus, CAMVE mediated cytotoxicity is more seen in U937 cells.

### Example 55

### CAMVE Induces Delayed Cell Cycle Progression

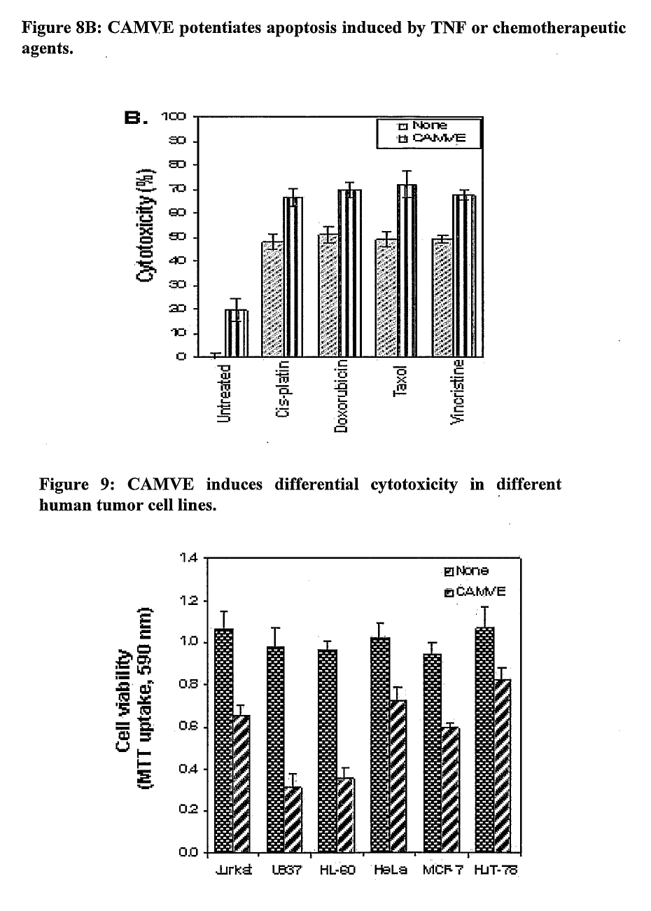

Polyphenolic compounds are also known to exert their anti-cancer properties by modulating cell cycle progression. To verify whether CAMVE modulates cell growth, we examined the effects of different concentrations of CAMVE on the cell cycle distribution of Jurkat cells. According to the cell cycle analysis, FIG. 10A shows that the treatment of Jurkat cells with as low as 5 μM dose of CAMVE for 24 h, resulted in a significant increase of cells in the G1 and compensatory decrease in the S phase of the cell cycle. These results suggest that CAMVE can induce a delayed cell cycle progression during G1/S transition, resulting in decreased cell proliferation rates.

The transition of G1/S phase is positively regulated by cell cycle regulatory proteins such as cyclin D1, cyclin E, cdk2 and cdk4 (Kwon, Y. K., Jun, J. M., Shin, S. W., Cho, J. W., Suh, S. I. (2004) International J of Oncology 26: 1597-1603, 2005). Cyclin D1 is a proto-oncogene that is over expressed in many cancer cell types and known to play a role in cell proliferation through activation of cyclin-dependent kinases (Mukhopadhyay, A., Banerjee, S., Stafford, L. J., Xia, C., Liu, M. and Aggarwal B B (2002) Oncogene 21: 8852-8861.). Because cyclin D1 plays important roles in progression of G1 phase into the S phase, we investigated the effect of CAMVE on the expression of cyclin D1 mRNA expression using RT-PCR analysis. When cells were treated with various concentrations of CAMVE for 18 h, the expression of cyclin D1 m-RNA was notably decreased as shown in FIG. 10B. Thus, downregulation of cyclin D1 mRNA by CAMVE leads to decreased cell proliferation, supporting the idea that CAMVE can also be used as a promising chemopreventive agent.

### Example 56

### Biological Evaluation of Compound 27

Cell Lines: The cell lines used in this study were as follows: Jurkat (human T cell leukemia), MCF-7 (human breast cancer cell line), U-937 (human histiocytic lymphoma) HeLa (human cervical cancer cell line); they were obtained from American Type culture collection (Manassas, Va., USA). L929, U-937, Jurkat was cultured in RPMI 1640, while others in DMEM supplemented with 10% FBS, penicillin (1000 U/ml), and streptomycin (100 μg/ml).

Materials: All synthetic chemicals were obtained from commercial sources. Propidium Iodide (PI), tetrazolium salt 3-(4,5-dimethylthiazol-2-yl)-2,5-diphenyltetrazolium bromide (MTT), caspase 3 substrate (Ac-DVED-pNA), DMSO etc were obtained from Sigma Aldrich Chemicals (St Louis, Mo., USA). Penicillin, streptomycin, neomycin, RPMI 1640 and DMEM medium, fetal bovine serum (FBS) were obtained from Gibco BRL., anti PARP-FITC conjugate was from Novus Biologicals, Monoclonal Anti-Phosphotyrosine FITC conjugate was from Sigma Aldrich (Saint Louis, Mo., USA). One step Access RT-PCR kit was purchased from Promega.

Cytotoxicity assay: Cytotoxicity was assayed by the modified tetrazolium salt 3-(4,5-dimethylthiazol-2-yl)-2,5-diphenyltetrazolium bromide (MTT) assay as described by Plumb, J A, et al (Cancer Res. 1989 Aug. 15; 49(16):4435-4440).

Cell Cycle Analysis: Cells (log phase culture) were treated with vehicles alone (similar volumes of DMSO) or with the compound (various concentrations) to be tested for 24 h. Untreated cells were also included in this experiment for comparison. After treatments, the cells were harvested and washed with cold PBS. Cells were then resuspended in cold PBS (300 μl) and 100% chilled ethanol (700 μl), vortexed, and incubated at room temperature for 1 h. Samples were centrifuged at 200×g for 5 minutes and the supernatant was removed. A solution containing propidium iodide (100 μg/ml) and RNase A (1 mg/ml) was added to the samples and incubated for 1 h at room temperature. Samples were then transferred to 12×75 mm polystyrene tubes and analyzed on flow cytometer. Flow cytometry analyses were done on FACSCalibur (Becton Dickinson, San Jose, Calif.) and data were analyzed using CellQuest analysis software.

Caspase 3 activity assay: To evaluate caspase 3 activity, cell lysates were prepared after their respective treatments with the compounds. 200 μg of the cell lysates were incubated with 50 μM caspase 3 substrate (Ac-DVED-pNA) in 100 μl reaction buffer (1% NP-40, 20 uM tris-HCl, pH7.5, 137 mM NaCl, and 10% glycerol) and incubated for 2 h at 37° C. The release of chromophore pNA was monitored spectrophotometrically at 405 nm.

PARP Cleavage Assay using Flow Cytometry: Extent of PARP cleavage is determined using polyclonal antibody specifically recognizing the 85 kDa fragment of cleaved PARP (NSB 699 Novus Biologicals) and can be used as a marker for detecting apoptotic cells. Treated cells were fixed with 70% chilled ethanol and permeabilized for 30 min at RT (PBS+0.5% BSA+0.02% NaN₃+0.5% saponin) and stained with anti PARP-FITC (10 μl/10⁶ cells) for one hour at RT. Cells were washed twice with wash buffer (PBS+1% heat inactivated FBS) and analysed using FACS.

DNA fragmentation Assay: After treatment with the compounds for 24 h, cells were harvested and washed in PBS. The cell pellet was incubated with lysis buffer (10 mM Tris-HCl pH 7.5, 1 mM EDTA, 1% SDS and 80 ug/ml proteinase K) at 37° C. overnight. After extraction with phenol/chloroform, the DNA was precipitated with 100% ethanol and then dissolved in Tris-EDTA buffer (pH 8.0) with RNase A at 37° C. The DNA estimation was performed by taking absorbance at 260/280 nm, and DNA was resolved in a 1.8% agarose gel, stained with ethidium bromide and visualized under a UV transilluminator.

Nuclear staining assay: After treatment with the compound for 24 h, Jurkat cells were washed once with ice-cold EDTA/PBS (5 mM) and fixed with 70% ethanol for 1 h at RT. Fixed cells were placed on slides and stained with PI (5 μg/ml) with RNase A (1 mg/ml) for 20 mins. After decolourization with water, nuclear morphology of cells was examined by fluorescence microscopy.

RNA extraction: Total RNA was isolated using the standard TRIzol method (Gibco BRL). Briefly, 3×10⁶ cells were treated with different concentrations of the compounds as indicated in the legends to Figures and after harvesting the cell pellet was resuspended in 1 ml of TRIzol with repeated pipetting. The homogenized sample was incubated at RT for 5 min to permit complete dissociation of the nucleoprotein complexes. 2000 of chloroform was added and the tubes were shaken vigorously for 15 sec by hand and then incubated at RT for 3 mins. The samples were then centrifuged at 10,000 RPM for 15 mins at 4° C. The aqueous phase was transferred to a fresh tube and 500 μl of Isopropyl alcohol was added, followed by incubation at RT for 10 mins. The samples were centrifuged at 10,000 RPM for 10 min at 4° C. and the RNA pellet was washed with 1 ml of 75% ethanol followed by centrifugation at maximum speed at 4° C. for 10 mins. Washing was repeated once more and after removing the supernatant, the RNA pellet was dried and dissolved in 20 ul of RNase free water (Promega). To ensure total resuspension, tubes were incubated at 55-60° C. for 10 mins. The samples were aliquoted and stored at −70° C.

Semi-quantitative reverse transcriptase (RT)-PCR: Changes in gene expression were verified by semi-quantitative RT-PCR using GAPDH as an internal normalization standard. 1 μg of total RNA (quantified by spectrophotometer) was used to reverse transcribe into cDNA. One step Access RT-PCR kit (Promega) was used for the synthesis of c-DNA followed by the amplification of the gene of interest using gene specific primers. Briefly, 50 μl reaction mixture including 10 μl of 5×AMV/Tfl reaction buffer, 0.2 mM dNTP mix, 50 pmol each of forward and reverse primers, 1 mM of MgSO₄ and 1 μg of RNA sample, was subjected to 28 PCR cycles (First strand cDNA synthesis: reverse transcription at 48° C. for 45 minutes, AMV RT inactivation and RNA/cDNA/primer denaturation at 94° C. for 2 minutes. Second strand synthesis and PCR amplification: denaturation at 94° C. for 30 sec, annealing at (primer specific temperature) for 1 minute and polymerization at 68° C. for 2 minutes. Amplification products were separated by agarose gel electrophoresis (2%) and visualized by ethidium bromide staining. The primer sequence and product size are as follows: (Louis, M., Rosato, R. R., Brault, L., Osbild, S., Battaglia, E., Yang, X. H., Grant, S. and Bagrel, D. (2004) Internat. J. of Oncology 25, 1701-1711)

| | |
|---|---|
| p53: 435 bp | |
| 5′-ATTCTGGGACAGCCAAGTCT-3′ | {forward} |
| 5′-GGAGTCTTCCAGTGTGATGA-3′ | {reverse} |
| bcl-2: 127 bp | |
| 5′-CTGTGGATGACTGAGTACCT-3′ | {forward} |
| 5′GAGACAGCCAGGAGAAATCA-3′ | {reverse} |
| Bax-α: 489 bp | |
| 5′-GTTTCATCCAGGATCGAGCA-3′ | {forward} |
| 5′-CCATCTTCTTCCAGATGGTG-3′ | {reverse} |
| GAPDH: 239 bp | |
| 5′-TGATGACATCAAGAAGGTGGTGAA-3′ | {forward} |
| 5′-TCCTTGGAGGCCATGTGGGCCAT-3′ | {reverse} |

Tyrosine Phosphorylation assay: Tyrosine Phosphorylation assay was performed by the method described by Far, D. F. et. al., (Cytometry (1994) 15(4):327-334) and Park. J. B., et al. ((2003) Cancer Letters, 202 161-171). Briefly, cells (10⁶) were washed with ice-cold PBS, pH 7.2 for 30 min at 4° C. After centrifugation and a PBS wash it was treated with 5 ml chilled 70% ethanol. The fixed cells were recovered by centrifugation followed by washing with PBS. The cells were permeabilized with saponin (0.05% in PBS) for 10 min at room temperature. Non specific binding was blocked by incubating the cells for 30 min in PBS, pH 7.6 containing BSA 0.1% and 0.1% (v/v) Tween 20. Thereafter the cells were stained with 20 μg/ml of FITC-conjugated anti-phosphotyrosine antibody for 30 min. Extent of tyrosine phosphorylation in the cells was determined by measuring the increase in fluorescence produced by the FITC-labeled monoclonal antibody compared to the FITC-labeled isotype control antibody. Fluorecence events for 10,000 cells were collected and analyzed by flow cytometry (FACSCalibur cytometer with CellQuest software, Becton Dicinson, San Jose, Calif.).

Activities Associated with Benzofuran Lignan Derivatives

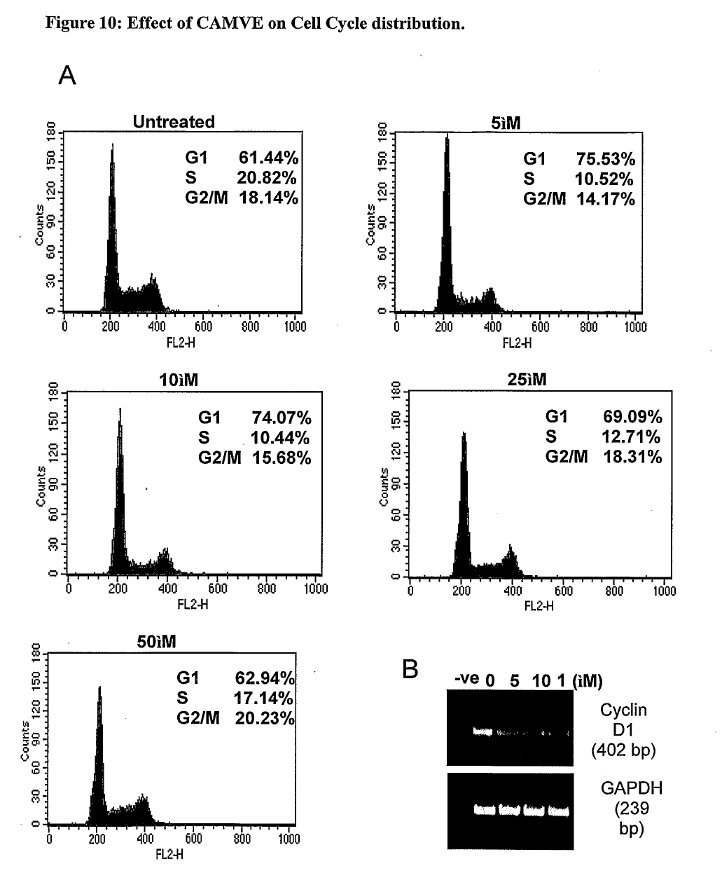

Cell viability and growth inhibition: Because benzofuran lignans has been reported to contain antiproliferation activity against human tumor cells, the activity of the benzofuran lignan derivative (compound 27) was investigated to determine whether it is capable of inhibiting cell growth of human cancer cells. Jurkat cells were used because this cell line has been used extensively for investigating growth proliferation and cell cycle progression in various cancer studies. The cells were treated for 24 h and 48 h with various concentrations of the compound. As shown in FIG. 11 the number of living cells decreased with the increasing concentration of the compound. The GI₅₀ representing the concentration of compound causing 50% growth inhibition compared with control cells is approximately around 100 nM.

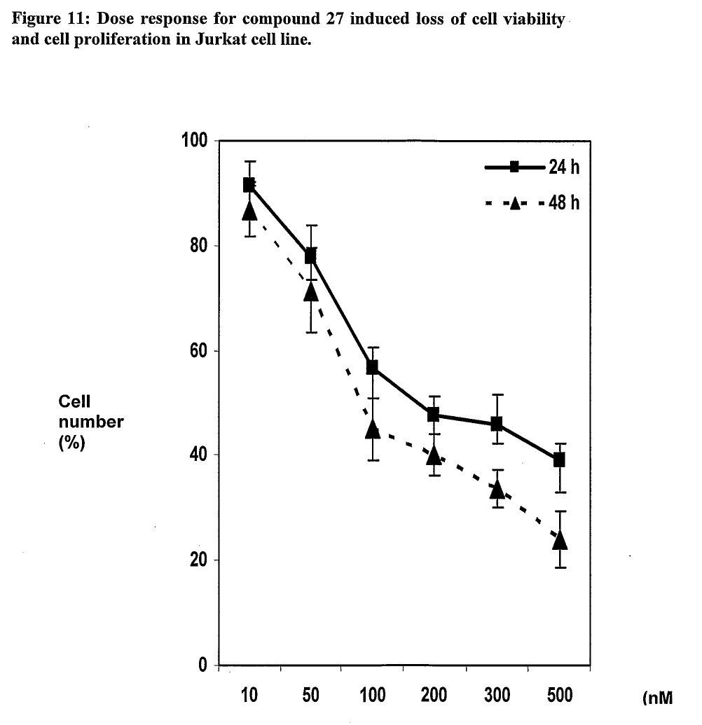

Effect on Cell Cycle: To understand the mechanism of action, this novel apoptosis-inducing compound was evaluated for its effect on cell cycle by measuring DNA content. We used flow cytometric analysis after treatment of Jurkat cells with varying doses of the compound. As shown in FIG. 12 compound specifically arrested cells in the G2-M phase of the cell cycle leading to significant apoptosis as shown in the sub-G1 content. The data shown here confirms that the compound dose as low as 100 nM is effective in significant increase (˜50%) of cells in the G2/M phase of the cell cycle. At 50 nM (data not shown) no significant increase in G2/M population was achieved. At concentration higher than 100 nM there was further increase in both G2/M and sub G1 population.

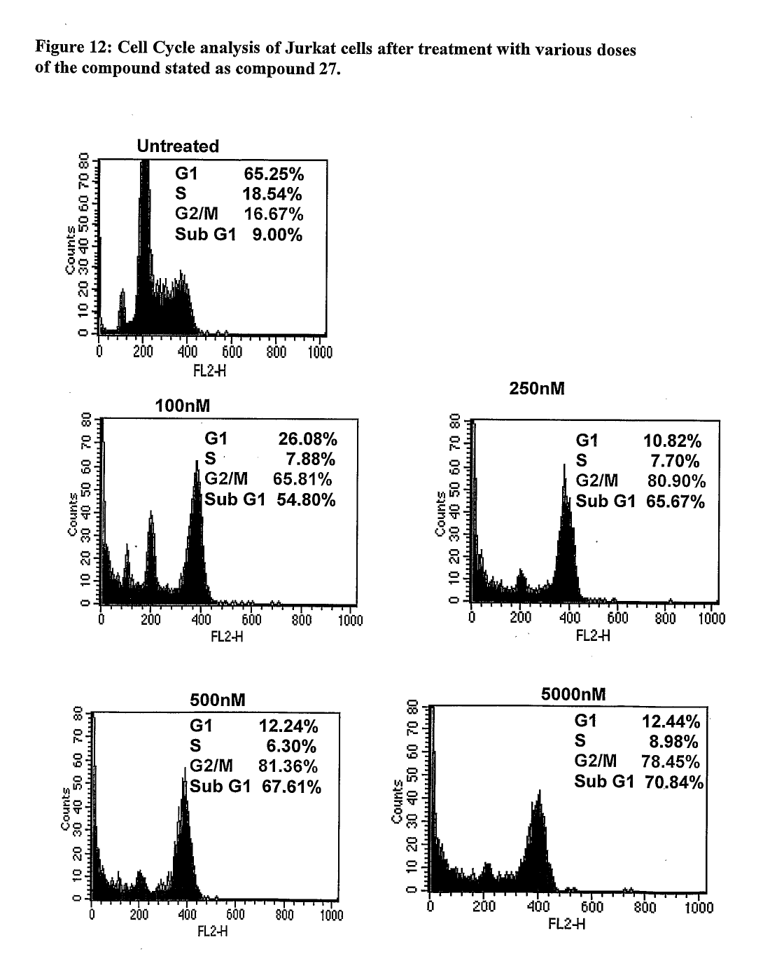

Time dependent effects of the G2/M promoting doses of compound 27 on the cell cycle distribution: The time-dependent effects on the cell cycle after treatment with 100 nM and 500 nM of the compound over 24 h, 48 h, 72 h time duration was evaluated. As shown in FIG. 13, cells treated with both the concentrations accumulated in G2/M after 24 h with a significant decrease in the G1 and S phase populations. However, after 48 and 72 h of treatment there was a significant decrease in the G2/M population in the cells treated with both doses, followed by an increase in the S and G1 phase population.

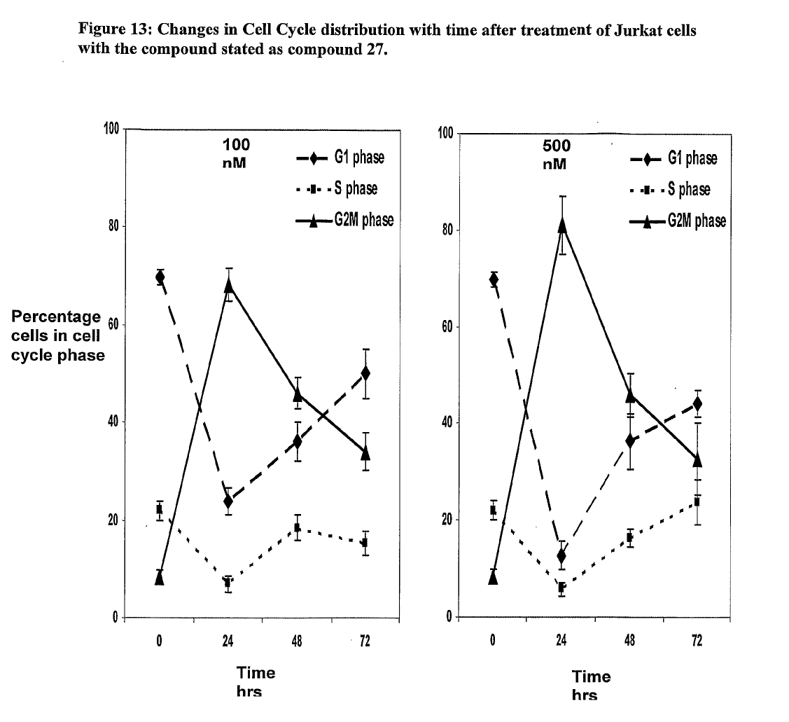

Compound 27 induced caspase activation: Caspases are important mediators of apoptosis induced by various apoptotic stimuli. Induction of cell death predominantly occurs after the G2/M cell cycle block in cancer cells. Cell death is related to cellular and molecular events in the cells and occur via two independent cell death processes i.e. necrosis and apoptosis (Park. J. B., Schoene, N. (2003) Cancer Letters, 202 161-171). Necrotic cell death is an accidental cell death that does not require any cellular and molecular mechanism and leads to inflammation and tissue injury. Apoptosis, however, does require programmed cellular or molecular events, such as activation of key proteases like caspases. In this study the proteolytic activity of caspase 3 was investigated and quantified by an in vitro assay based on the proteolytic cleavage of DVED-pNA by caspase 3 into the pNA. As shown in FIG. 14, Jurkat cells demonstrated a dose dependent increase in DVED-pNA cleavage after 16 and 24 h exposure to the compound. The activity at 24 h was more than the activity at 16 h. This data clearly suggests that G2/M arrest induces caspase activation in cells after treatment with the compound.

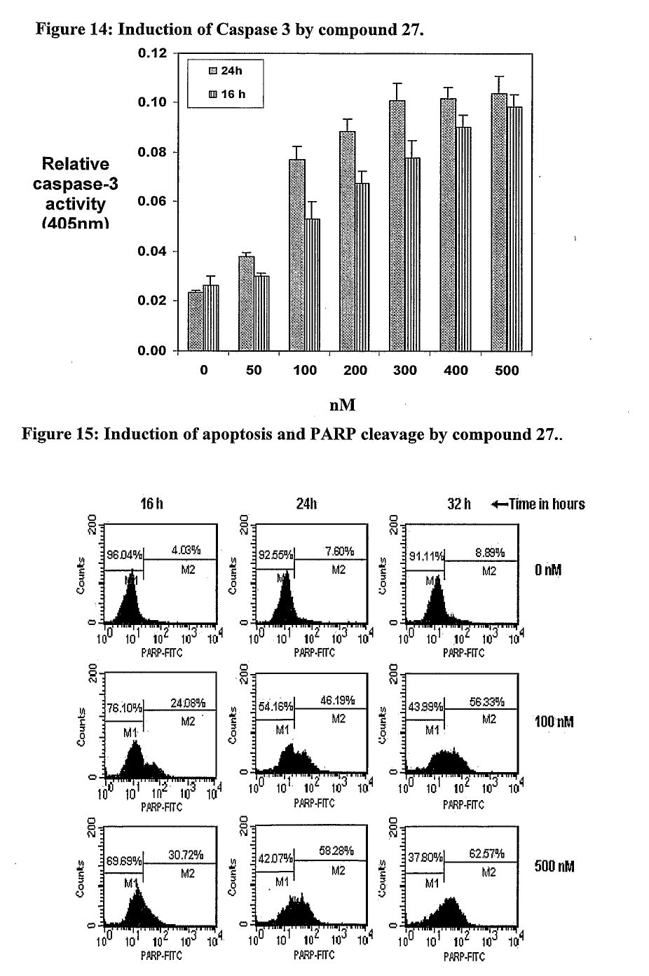

Compound 27 induced PARP cleavage and apoptosis is time and dose dependent: An important factor in inducing apoptosis is the enzyme Poly (ADP-ribose) polymerase (PARP) that has been widely studied in vitro. An early transient burst poly (ADP-ribosylation of nuclear proteins was recently shown to be required for apoptosis to proceed in various cell lines followed by cleavage of poly (ADP-ribose) polymerase (PARP), catalyzed by caspases. As shown in FIG. 15, significant PARP cleavage was achieved at 100 nM and 500 nM and degree of PARP staining increased with time. The PARP cleavage data is very much consistent with the caspase 3 data. Magnitude of caspase 3 activation and sub G1 apoptotic population positively correlates to the degree of PARP cleavage. These data suggests strongly that the compound induces cell death via apoptotic processes.

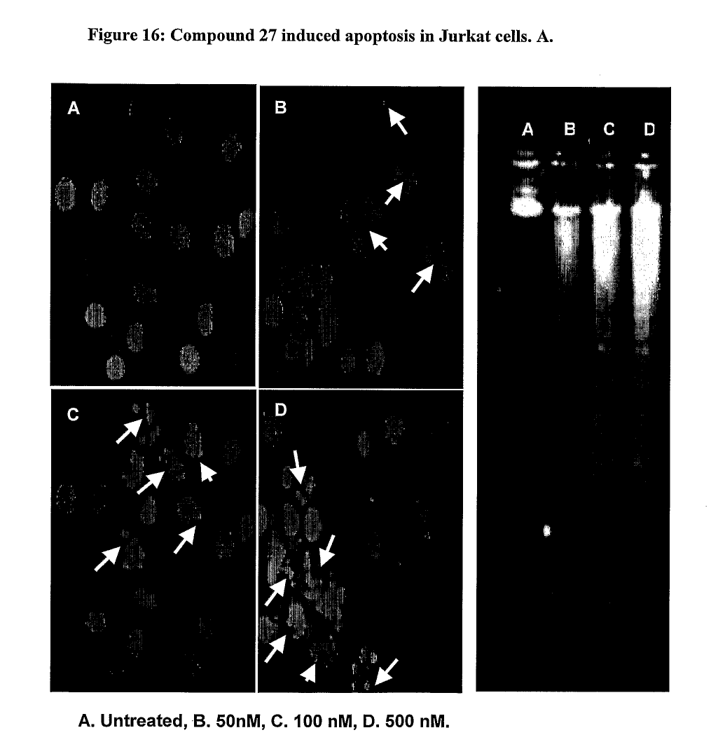

Compound 27 induced apoptosis in Jurkat cells: To assess the nature of apoptosis induced by compound 27, cells treated for 24 h with different concentrations of the compound were examined for their nuclear morphology after propidium iodide staining. As shown in FIG. 16. nucleic acid staining with propidium iodide revealed typical apoptotic nuclei in compound treated cells, but control cells did not show any features of apoptosis. Another hallmark of apoptosis is the degradation of chromosomal DNA at internucleosomal linkages. DNA fragmentation induced by the compound in Jurkat cells was analyzed. Following agarose gel electrophoresis of Jurkat cells treated with various concentrations of the compound for 24 h, a typical ladder pattern of internucleosomal fragmentation was observed (FIG. 16). Also the extent of apoptosis was analyzed from the cell cycle data (FIG. 12), showing a markedly increased accumulation of sub G1 phase population.

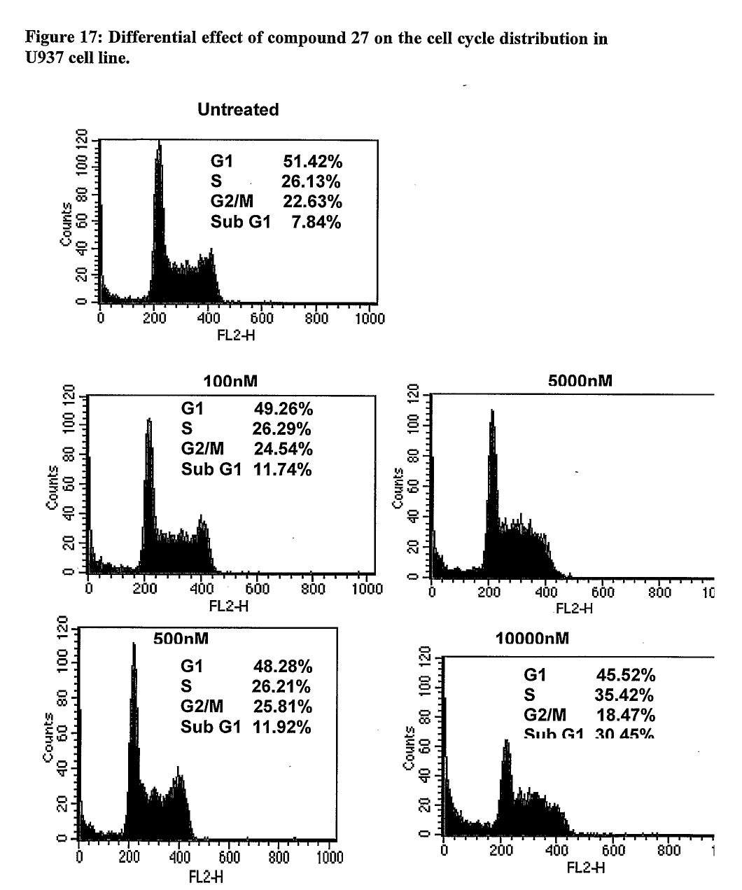

Compound 27 differential cell cycle arrest in cells with different p53 status: Since p53 is known to control the G2/M checkpoint, cell differentiation and apoptosis (Schwartz, D., Almog, N., Peled, A., Goldfinger, N. and Rotter, V (1997) Oncogene, 15, 2597-2607), the compound was tested for activation of a p53 dependent pathway. In order to compare the effects of p53 on compound sensitivity in tumor cell lines, we next assessed the response to the compound in U937 cells known to have mutant inactive p53 status. U937 cells were treated for 24 h with 100, 500, 5000, 10000 nM of the compound and cell cycle analysis was performed. As shown in FIG. 17, no significant increase in the proportion of cells in the G2/M phase of the cell cycle was seen at any of the doses. There was also no increase in the sub 01 population of the cells treated with 100 and 500 nM of the compound, clearly stating that these doses were unable to modulate the G2/M checkpoints or cause apoptosis in U937 cell line. However, at higher doses i.e. 5 and 10 μM, there was an increase in the proportion of cells in the S phase of the cell cycle (from 26%-35%) accompanied by a slight compensatory decrease in G1 phase cells. At 10 μM, the sub G1 population increased to almost 30% indicating apoptosis. Thus, these data suggests that the of accumulation of Jurkat cells in the G2/M phase after treatment with lower doses of the compound may indicate that this checkpoint is operational at the G2/M border in Jurkat cells, whereas this checkpoint is not operational in the U937 cell line. This checkpoint may be p53 related, because Jurkat cells have normal p53, whereas U937 cells have mutant inactive p53. However, at higher doses, U937 cells shows a S phase arrest and induction of apoptosis, probably in a p53 independent fashion.

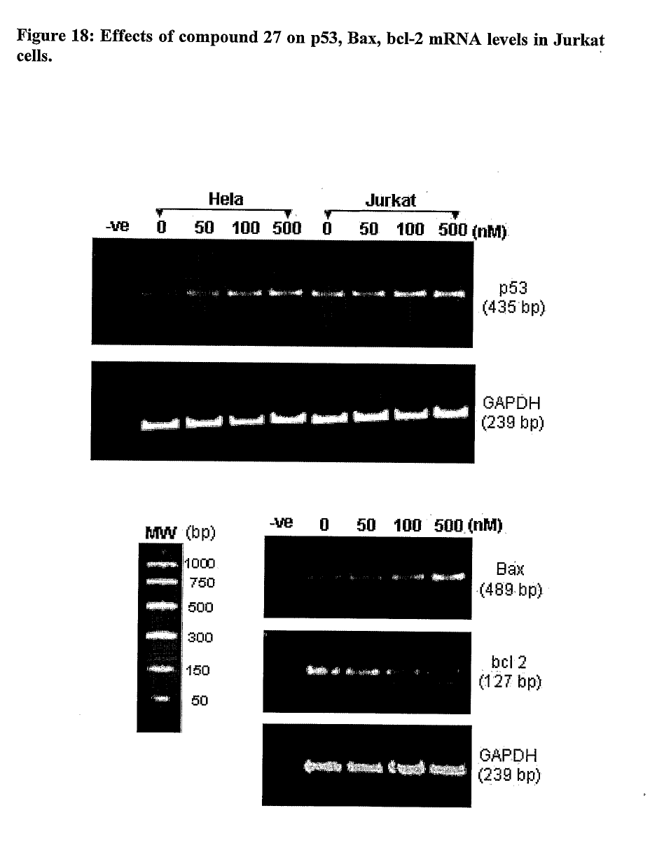

Effect of compound 27 on p53 mRNA expression: Given the relevance of p53, to the development of the cell cycle arrest and apoptotic response, we next examined it's response to the compound by semi-quantitative RT-PCR in two cell lines with different p53 status. The sensitivity of gene expression in Jurkat (wild type p53) and HeLa (very low level of p53) after treatment with various doses of the compound for 12 h is shown in FIG. 18 A. In HeLa cells, significant induction of p53 transcription was seen in a dose dependent fashion. However, there was only slight increase in p53 mRNA level in case of Jurkat cell line. The p53 proapoptotic factor acts as a transcriptional regulator for many genes and could effect the transcription of some of the other genes involved in the apoptotic pathway (Mansilla, S., Pina, B., Portugal, J. (2003) Biochem. J. 372, 703-711).

Effects on p53 regulated mRNA expression of bcl-2 and bax: p53 is known to regulate the expression of the apoptosis regulating proteins. We investigated the expression of the pro and anti-apoptotic proteins Bax and Bcl-2 in compound 27 treated cells. As shown in FIG. 18B, semi-quantitative RT-PCR analysis indicated that the anti-apoptotic bcl-2 mRNA levels were downregulated in a dose dependent manner by the compound 27. On the other hand, exposure to increasing concentrations of the compound increased the pro-apoptotic bax mRNA levels in Jurkat cells.

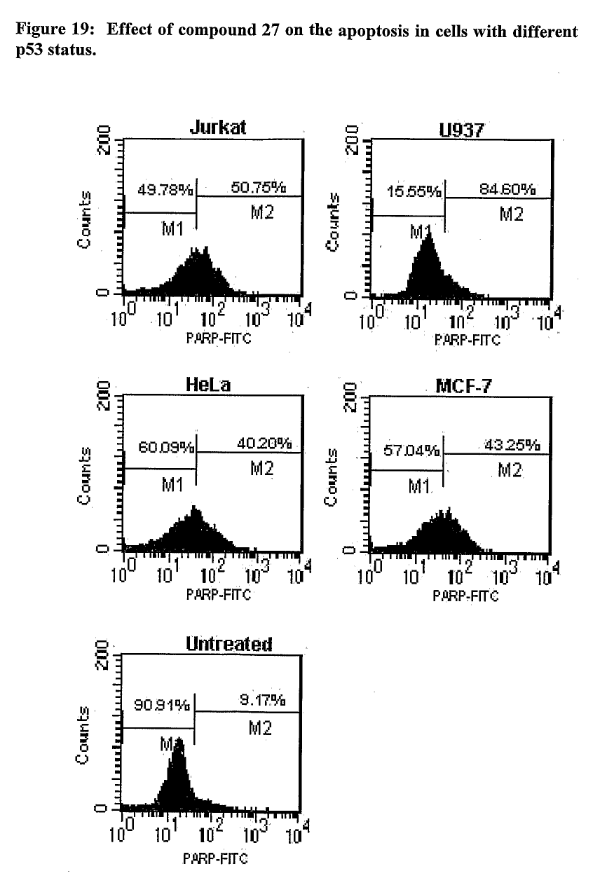

Compound 27 induced differential levels of apoptosis in cells with different p53 status: To prove that the growth arrest and apoptosis of cancer cells caused by the compound were indeed p53 dependent, we investigated the extent of apoptosis in different cell lines having different p53 status. As shown in FIG. 19, the level of apoptosis in MCF-7 was comparable to that of Jurkat cell line. On the other hand HeLa cells known to have very low levels of p53 expression also shows significant induction of apoptosis after treatment with 100 nM of the compound for 24 h. This observation is positively correlated with the data shown in FIG. 18 A, stating that the compound treatment also leads to an induction in p53 mRNA expression levels in the HeLa cell line. U937 cell line having mutant inactive p53 status does not show induction of apoptosis with the treatment of this compound. Thus, this data clearly proves the significance of p53 in controlling the G2/M checkpoint and subsequent induction of apoptosis. Most of the breast and lung tumors are known to have wild type p53 expression, hence compound 27 would be effective for the killing of such tumor cells.

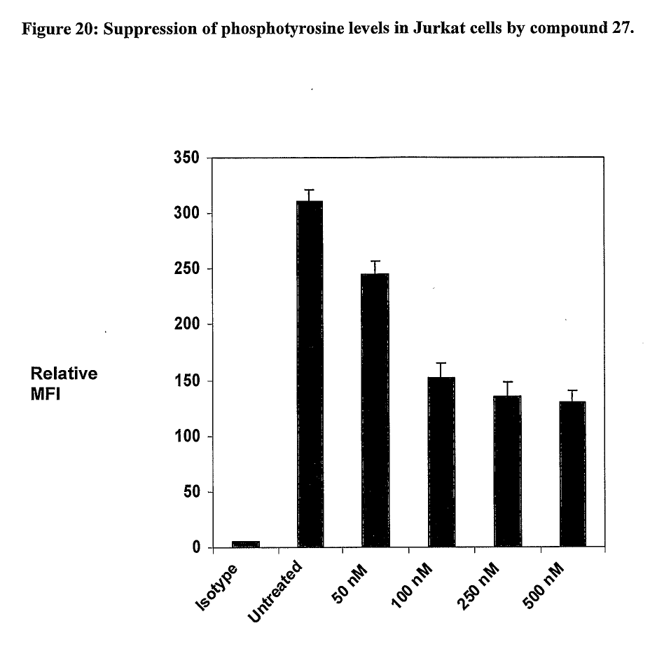

Suppression of constitutive tyrosine phosphorylation by compound 27: Without being bound by theory, the following represents and possible mode of action for the benzofuran derivatives of the invention. The phosphorylation of tyrosine residues of proteins is assumed to be involved in abnormal growth of human tumor cells (Lui, V. W., Grandis, J. R. (2002)Anticancer Res. 22, 681-690.). As reported earlier, chemotherapeutic compounds may act partly through inhibition of protein tyrosine phosphorylation to arrest cell cycle progression and induce apoptosis (Chen, H. W., Huang, H. C. (1998) British J. Pharmacology 124, 1029-1040). Since leukemic and breast cancer cells are reported to have a high level of constitutive phosphotyrosine levels, the effect of this compound on the inhibition of protein tyrosine phosphorylation was investigated. Total tyrosine phosphorylation in Jurkat cells was determined by flow cytometry with FITC-labeled monoclonal antibody against phosphotyrosine. As shown in FIG. 20, the phosphotyrosine level was reduced at all doses. At 100 nM dose itself, significant reduction in the mean fluorescence intensity was achieved when compared with the untreated controls. This data suggests that the ability of the compound to reduce the constitutive phosphotyrosine levels could be one of the mechanisms in regulating cell proliferation and causing cell death. As previously reported (Chen, Z. P., Yeung, D. C. (1996) Biochem Mol Biol Int. 38(3):607-616), significant induction of p53 message was seen when phosphotyrosine levels were reduced in HeLa cells, giving an additional explanation for the induction p53 expression and regulation of genes involved in cell cycle checkpoints and apoptosis of tumor cells.

All publications and patent applications cited in this specification are herein incorporated by reference as if each individual publication or patent application was specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention.

1. A pharmaceutical composition comprising a therapeutically effective amount of a compound of formula I which is an ester derivative of cinnamic acid: 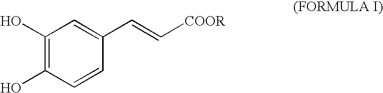
wherein R is selected from the group consisting of aryl and hetero-aryl, and derivatives, polymorphs, isomers, prodrugs, geometric isomers, optical isomers, esters, ethers, carbamates, solvates, hydrates, and salts thereof; and
a pharmaceutically acceptable excipient.

2. The pharmaceutical composition according to 1 wherein R is selected from vannilic acid, ferulic acid, eugenol, salicylic acid and derivatives thereof in the compound of formula I.

3. A compound of formula I selected from the group consisting of:
Methyl 4-{[(2E)-3-(3,4-dihydroxyphenyl)prop-2-enoyl]oxy}-3-methoxy benzoate (CAMVE; Compound 1);
2-methoxy-4-[(1E)-3-methoxy-3-oxoprop-1-en-1-yl]phenyl (2E)-3-(3,4 dihydroxy phenyl)acrylate (Compound 2);
Methyl 2-{[(2E)-3-(3,4-dihydroxyphenyl)prop-2-enoyl]oxy}benzoate (Compound 3);
4-Allyl-2-methoxyphenyl (2E)-3-(3,4-dihydroxyphenyl)acrylate (Compound 4);
(±)-2β-[4-O-(3,4,-dihydroxycinnamyl)-3-methoxyphenyl]-3α-methyl-7-methoxy-5-[(E)-1-propenyl]-2,3-dihydrobenzofuran (Compound 5);
Methyl(E)-3-[2β-{4-O-[3,4-dihydroxycinnamyl)-3-methoxyphenyl}-7-methoxy-3α-methoxycarbonyl-2,3-dihydro-1-benzofuran-5-yl]propen-2-enate (Compound 6);
2-Methoxy-4-[(1E)-prop-1-en-1-yl]phenyl (2E)-3-(3,4-dihydroxyphenyl)acrylate (Compound 7);
4-Formyl-2-methoxyphenyl (2E)-3-(3,4-dihydroxyphenyl)acrylate (Compound 8); and
derivatives, polymorphs, isomers, prodrugs, geometric isomers, optical isomers, esters, ethers, carbamates, solvates, hydrates, and salts thereof.

4. The pharmaceutical composition according to 1 wherein the compound of formula I is selected from any one of compounds 1 through 8.

5. A pharmaceutical composition comprising a therapeutically effective amount of a compound of formula II which is an ester derivative of vanillic acid: 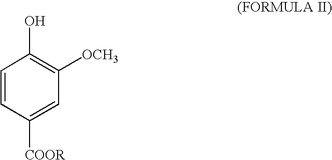
wherein R is selected from the group consisting of aryl and hetero-aryl, and derivatives, polymorphs, isomers, prodrugs, geometric isomers, optical isomers, esters, ethers, carbamates, solvates, hydrates, and salts thereof; and
a pharmaceutically acceptable excipient.

6. The pharmaceutical composition according to 4 wherein R is selected from vannilic acid, ferulic acid, eugenol, salicylic acid and derivatives thereof in the compound of formula II.

7. A compound of formula II selected from the group consisting of:
4-(Methoxycarbonyl)phenyl 4-hydroxy-3-methoxybenzoate (Compound 9);
2-Methoxy-4-(methoxycarbonyl)phenyl 4-hydroxy-3-methoxybenzoate (Compound 10);
2-Methoxy-4-[(1E)-3-methoxy-3-oxoprop-1en-1yl]phenyl 4hydroxy-3methoxy benzoate (Compound 11);
Methyl(E)-3-[2β-{4-O-(3-methoxy-4-hydroxyphenyl carbonyl)-3-methoxyphenyl}-7-methoxy-3α-methoxycarbonyl-2,3-dihydro-1-benzofuran-5yl]prop-2-enoate (Compound 12);
4-Allyl-2-methoxyphenyl 4-hydroxy-3-methoxybenzoate (Compound 13);
2-Methoxy-4-[(1E)-prop-1en-1-yl]phenyl 4-hydroxy-3-methoxybenzoate (Compound 14);
4-Formyl-2-methoxyphenyl 4-hydroxy-3-methoxybenozoate (Compound 15);
(±)-2β-[4-O-(3-Hydroxy-4-methoxy)-3-methoxyphenyl]-3α-methyl-7-methoxy-5-[(E)-1-propenyl]-2,3-dihydrobenzofuran (Compound 16); and
derivatives, polymorphs, isomers, prodrugs, geometric isomers, optical isomers, esters, ethers, carbamates, solvates, hydrates, and salts thereof.

8. The pharmaceutical composition according to 4 wherein the compound of formula I is selected from any one of compounds 9 through 16.

9. A process for preparation of an ester derivative of formula I or II, the process comprising the steps of:
esterifying vanillic acid;
protecting all hydroxyl groups in the esterified vanillic acid as methoxymethyl (MOM) ether derivatives;
hydrolysis to generate a corresponding acid;
reacting the corresponding acid with a phenolic compound to generate a corresponding fused ester derivative; and
deprotecting of the hydroxyl groups using methanolic HCl to yield the ester derivative of formula II.

10. A compound obtained by the process of 9.

11. A pharmaceutical composition comprising a therapeutically effective amount of a compound of formula I which is an ester derivative of 4-hydroxy cinnamic acid: 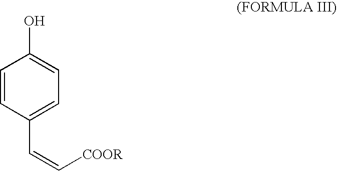
wherein R is selected from the group consisting of aryl and hetero-aryl, and derivatives, polymorphs, isomers, prodrugs, geometric isomers, optical isomers, esters, ethers, carbamates, solvates, hydrates, and salts thereof; and
a pharmaceutically acceptable excipient.

12. The pharmaceutical composition according to 11 wherein R is selected from vannilic acid, ferulic acid, eugenol, cinnamic acid, salicylic acid and derivatives thereof in the compound of formula III.

13. A compound of formula I selected from the group consisting of:
Methyl 4-{[(2E)-3-(4-hydroxyphenyl)prop-2-enoyl]oxy}-3-methoxybenzoate (Compound 17);
2-methoxy-4-[(1E)-3-methoxy-3-oxoprop-1-en-1-yl]phenyl(2E)-3-(4-hydroxy phenyl)acrylate (Compound 18);
4-Formyl-2-methoxyphenyl (2E)-3-(4-hydroxyphenyl)acrylate (Compound 19);
2-Methoxyphenyl (2E)-3-(4-hydroxyphenyl)acrylate (Compound 20);
4-Allyl-2-methoxyphenyl (2E)-3-(4-hydroxyophenyl)acrylate (Compound 21);
Methyl [3,4-bis O-(4-hydroxyphenylacryloyl)]phenylacrylate (Compound 22);
2-Methoxy-4-(1E)-prop-1en-1yl]phenyl (2e)-3-(4-hydroxyphenyl)acrylate (Compound 23);
Methyl (E)-3-[2β-{4-O-(4-hydroxycinnamoyl)-3-methoxyphenyl}-7-methoxy-3α-methoxycarbonyl-2,3-dihydro-1-benzofuran-5-yl-prop-2-enoate (Compound 24); and
derivatives, polymorphs, isomers, prodrugs, geometric isomers, optical isomers, esters, ethers, carbamates, solvates, hydrates, and salts thereof.

14. The pharmaceutical composition according to 11 wherein the compound of formula I is selected from any one of compounds 17 through 24.

15. A process for preparation of an ester derivative of formula III, the process comprising the steps of:
esterifying 4-hydroxy cinnamic acid;
protecting all hydroxyl groups in the esterified 4-hydroxy cinnamic acid as methoxymethyl (MOM) ether derivatives;
hydrolysis to generate a corresponding acid;
reacting the corresponding acid with a phenolic compound to generate a corresponding fused ester derivative; and
deprotecting of the hydroxyl groups using methanolic HCl to yield the ester derivative of formula III.

16. A compound obtained by the process of 15.

17. A compound having a benzofuran lignan structure selected from the group consisting of:
(±)-2β-{4-O-(3-methoxy-4-hydroxy cinnamoyl)-3-methoxyphenyl]-3α-methyl-7-methoxy-5-[(E)-1-propenyl]-2,3-dihydrobenzofuran (Compound 25);
2-methoxy-4-(methoxycarbonyl)phenyl 3,4,5-trihydroxybenzoate (Compound 26);
5-[(E)-2-carboxyvinyl]-2β-(4-hydroxy-3-methoxyphenyl)-7-methoxy-2,3-dihydro-1-benzofuran-3α-carboxylic acid (Compound 27);
5-[(E)-2-carboxyvinyl]-7-hydroxy-2β-(4-hydroxy-3-methoxy phenyl)-2,3-dihydro-1-benzofuran-3α-carboxylic acid (Compound 28);
(±)-2β-[4-O-(4-hydroxy cinnamoyl)-3-methoxyphenyl]-3α-methyl-7-methoxy-5-[(E)-1-propenyl]-2,3-dihydrobenzofuran (Compound 29), and
derivatives, polymorphs, isomers, prodrugs, geometric isomers, optical isomers, esters, ethers, carbamates, solvates, hydrates, and salts thereof.

18. A pharmaceutical composition comprising a therapeutically effective amount of a compound selected from any one of compounds 25 through 29.; and
a pharmaceutically acceptable excipient.

19. A process for preparation of a compound comprising a benzofuran lignan structure, the process comprising the steps of:
reacting boron tribromide with Methyl (E)-3-[2-(4-hydroxy-3-methoxyphenyl)-7-methoxy-3-methoxycarbonyl-2,3-dihydro-1-benzofuran-5-yl]prop-2-enoate in dichloromethane under appropriate conditions;
decomposing the reaction mixture with water;
washing an organic layer therefrom with saturated solution of sodium bicarbonate, water, and brine;
concentrating the organic layer by contacting with anhydrous sodium sulphate to yield a crude mass of a compound comprising a benzofuran lignan structure; and
optionally, purifying the crude mass by radial chromatography with increasing concentrations of ethyl acetate in petroleum ether.

20. A compound obtained by the process of 19.

21. A compound according to any one of 17 and 20 wherein the compound has an apoptotic, antimitotic, antitumor or antiproliferative activity.

22. A compound according to any one of formula I, formula II, formula III or any one of compounds 1-29, or a compound according to 10, 16, or 20, wherein the compound modulates NF-kappaB activity or expression.

23. A pharmaceutical formulation suitable for treating a disease or condition by modulating NF-kappaB activity comprising a therapeutically effective amount of a compound of 22; and
a pharmaceutically effective excipient.

24. A pharmaceutical formulation comprising an effective amount of a compound of 22 sufficient to cause cell cycle arrest.

25. A pharmaceutical formulation suitable for treating a disease or condition associated with inflammation comprising a therapeutically effective amount of a compound of 22; and
a pharmaceutically effective excipient.

26. A pharmaceutical formulation suitable for treating cancer comprising:
a therapeutically effective amount of a compound of 22; and
at least one anticancer drug selected from the group consisting of: Acivicin®; Aclarubicin®; Acodazole Hydrochloride®; Acronine®; Adozelesin®; Aldesleukin®; Altretamine®; Ambomycin®; Ametantrone Acetate®; Aminoglutethimide®; Amsacrine®; Anastrozole®; Anthramycin®; Asparaginase®; Asperlin®; Azacitidine®; Azetepa®; Azotomycin®; Batimastat®; Benzodepa®; Bicalutamide®; Bisantrene Hydrochloride®; Bisnafide Dimesylate®; Bizelesin®; Bleomycin Sulfate®; Brequinar Sodium®; Bropirimine®; Busulfan®; Cactinomyde®; Calusterone®; Caracemide®; Carbetimer®; Carboplatin®; Carmustine®; Carubicin Hydrochloride®; Carzelesin®; Cedefingol®; Chlorambucil®; Cirolemycin®; Cisplatin®; Cladribine®; Crisnatol Mesylate®; Cyclophosphamide®; Cytarabine®; Dacarbazine®; Dactinomycin®; Daunorubicin Hydrochloride®; Decitabine®; Dexormaplatin®; Dezaguanine®; Dezaguanine Mesylate®; Diaziquone®; Docetaxel®; Doxorubicin®; Doxorubicin Hydrochloride®; Droloxifene®; Droloxifene Citrate®; Dromostanolone Propionate®; Duazomycin®; Edatrexate®; Eflornithine Hydrochloride®; Elsamitrucin®; Enloplatin®; Enpromate®; Epipropidine®; Epirubicin Hydrochloride®; Erbulozole®; Esorubicin Hydrochloride®; Estramustine®; Estramustine Phosphate Sodium®; Etanidazole®; Etoposide®; Etoposide Phosphate®; Etoprine®; Fadrozole Hydrochloride®; Fazarabine®; Fenretinide®; Floxuridine®; Fludarabine Phosphate®; Fluorouracil®; Fluorocitabine®; Fosquidone®; Fostriecin Sodium®; Gemcitabine®; Gemcitabine Hydrochloride®; Hydroxyurea®; Idarubicin Hydrochloride®; Ifosfamide®; Ilmofosine®; Interferon Alfa-2a®; Interferon Alfa-2b®; Interferon Alfa-n1®; Interferon Alfa-n3®; Interferon Beta-I a®; Interferon Gamma-I b®; Iproplatin®; Irinotecan Hydrochloride®; Lanreotide Acetate®; Letrozole®; Leuprolide Acetate®; Liarozole Hydrochloride®; Lometrexol Sodium®; Lomustine®; Losoxantrone Hydrochloride®; Masoprocol®; Maytansine®; Mechlorethamine Hydrochloride®; Megestrol Acetate®; Melengestrol Acetate®; Meiphalan®; Menogaril®; Mercaptopurine®; Methotrexate®; Methotrexate Sodium®; Metoprine®; Meturedepa®; Mitindomide®; Mitocarcin®; Mitocromin®; Mitomalcin®; Mitomycin®; Mitosper®; Mitotane®; Mitoxantrone Hydrochloride®; Mycophenolic Acid®; Nocodazole®; Nogalamycin®; Ormaplatin®; Oxisuran®; Paclitaxel®; Pegaspargase®; Peliomycin®; Pentamustine®; Peplomycin Sulfate®; Perfosfamide®; Pipobroman®; Piposulfan®; Piroxantrone Hydrochloride®; Plicamycin®; Plomestane®; Porfimer Sodium®; Porfiromycin®; Prednimustine®; Procarbazine Hydrochloride®; Puromycin®; Puromycin Hydrochloride®; Pyrazofurin®; Riboprine®; Rogletimide®; Safingol®; Safingol Hydrochloride®; Semustine®; Simtrazene®; Sparfosate Sodium®; Sparsomycin®; Spirogermanium Hydrochloride®; Spiromustine®; Spiroplatin®; Streptonigrin®; Streptozocin®; Sulofenur®; Talisomycin®; Taxol®; Taxotere®; Tecogalan Sodium®; Tegafur®; Teloxantrone Hydrochloride®; Temoporfin®; Teniposide®; Teroxirone®; Testolactone®; Thiamiprine®; Thioguanine®; Thiotepa®; Tiazofurin®; Tirapazamine®; Topotecan Hydrochloride®; Toremifene Citrate®; Trestolone Acetate®; Triciribine Phosphate®; Trimetrexate®; Trimetrexate Glucuronate®; Triptorelin®; Tubulozole Hydrochloride®; Uracil Mustard®; Uredepa®; Vapreotide®; Verteporfin®; Vinblastine Sulfate®; Vincristine Sulfate; Vindesine®; Vindesine Sulfate®; Vinepidine Sulfate®; Vinglycinate Sulfate®; Vinleurosine Sulfate®; Vinorelbine Tartrate®; Vinrosidine Sulfate®; Vinzolidine Sulfate®; Vorozole®; Zeniplatin®; Zinostatin®; Zorubicin Hydrochloride®, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminol evulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; aza osine; baccatin III derivatives; balanol; batimastat; BCR/ABL antagonists; benzochlorins; benzoylstaurosporine; beta-lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti cancer compound; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O₆-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, antimetabolites, platinum-based agents, alkylating agents, tyrosine kinase inhibitors, anthracycline antibiotics, vinca alkloids, proteasome inhibitors, macrolides, and topoisomerase inhibitors.

27. A pharmaceutical formulation according to any one of 23 to 26 further comprising at least one more therapeutically effective compound.

28. A pharmaceutical formulation according to any one of 23 to 27, wherein the formulation is suitable for administration by oral, parenteral, enteral, intraperitoneal, topical, transdermal, ophthalmic, nasally, local, non-oral, aerosol, inhalation, subcutaneous, intramuscular, buccal, sublingual, rectal, vaginal, intra-arterial, or intrathecal route.

29. A sealed vial comprising an unit dosage of a pharmaceutical formulation according to any one of 23 to 27.

30. The sealed vial according to 29 comprising a sub-therapeutic dosage of a pharmaceutical formulation according to any one of 23 to 27 for use in metronomic administration.

31. Use of a compound according to 22 for preparing a medicament for treatment of an NF-kappaB related disease or condition.

32. The use of 31 wherein the disease or condition is cancer or inflammation.

33. The use of 32 wherein the cancer is selected from the group consisting of breast, ovary, testicle, prostate, head, neck, eye, skin, mouth, throat, esophagus, chest, bone, lung, colon, sigmoid, rectum, stomach, kidney, liver, pancreas, brain, intestine, heart, adrenal cancer and neoplastic disease.

34. A method for treating an inflammatory disease, the method comprising administering a pharmaceutical composition according to any one of 23 and 27 to a patient in need thereof.

35. A method for treating cancer, the method comprising administering a pharmaceutical composition according to any one of 24 through 27 to a patient in need thereof.

36. A method of treating a proliferative disease in an individual comprising administering to the individual:
a) a therapeutically effective amount of a composition comprising a compound according to 22, and
b) an effective amount of at least one other chemotherapeutic agent, wherein said chemotherapeutic agent is selected from the group consisting of antimetabolites, platinum-based agents, alkylating agents, tyrosine kinase inhibitors, anthracycline antibiotics, vinca alkloids, proteasome inhibitors, macrolides, and topoisomerase inhibitors.

37. A method of treating a tumor in an individual comprising:
a) a first therapy comprising administering to the individual a therapeutically effective amount of a composition comprising a compound according to 22, and
b) a second therapy comprising chemotherapy, radiation therapy, surgery, or combinations thereof.

38. The compounds as per formula I, II, III and its compositions and use as per preceding claims substantially described herein exemplified herein substantially in the examples and figures.
